(19) United States
(12) Patent Application Publication
(10) Pub. No.: US 2025/0032680 A1
(43) Pub. Date: Jan. 30, 2025

(54) ANTIVIRAL AND ANTIMICROBIAL COATINGS AND METHODS THEREOF

(71) Applicant: IONOMR INNOVATIONS, INC., Vancouver (CA)

(72) Inventors: Benjamin Britton, Vancouver (CA); Heidi M. Britton, Delta (CA); Bobak Gholamkhass, North Vancouver (CA); Timothy J. Peckham, Vancouver (CA)

(73) Assignee: IONOMR INNOVATIONS, INC., Vancouver, BC (CA)

(21) Appl. No.: 18/551,105

(22) PCT Filed: Mar. 21, 2022
(86) PCT No.: PCT/CA2022/050417

§ 371 Date: Sep. 18, 2023

Related U.S. Application Data
(60) Provisional application No. 63/163,605, filed on Mar. 19, 2021.

(51) Int. Cl. A61L 31/10; A01N 43/52; A01P 1/00; A61L 29/08; A61L 29/16; A61L 31/16

(52) U.S. Cl. CPC A61L 31/10; A01N 43/52; A01P 1/00; A61L 29/085; A61L 29/16; A61L 31/16; A61L 2300/208; A61L 2300/404; A61L 2300/606

(57) ABSTRACT

The present disclosure features a method of inhibiting microorganisms on a surface, including applying to the surface a polymer including a repeating unit, the repeating unit including: a benzimidazolium-containing moiety; an imidazolium-containing moiety; or any combination thereof.

### CROSS-REFERENCE TO RELATED APPLICATION

This application claims the benefit of U.S. Patent Application No. 63/163,605, filed Mar. 19, 2021, the disclosure of which is incorporated herein by reference in its entirety.

### BACKGROUND

Antiviral and antimicrobial compounds are chemical compounds that reduce or mitigate the growth or development of viral and microbial organisms, respectively. The use of antiviral and antimicrobial compounds leads to either death or arrested growth of the targeted microorganisms, which include viruses and microbes.

Minimizing infections caused by pathogenic microorganisms is a great concern in many fields, such as in medical devices, hospital surfaces/furniture, dental restoration and surgery equipment, healthcare products and hygienic applications, water purification systems, textiles, food packaging and storage, industrial or domestic appliances, aeronautics, etc.

Infections are produced by touching, eating, drinking, or breathing substances that contains a pathogen. Generally, these infections are combated with agents that target the pathogen. Particularly problematic, however, are the microorganisms that can rapidly and easily mutate their genes to become resistant to these agents, making their elimination difficult. For instance, Staphylococcus aureus (S. aureus) commonly colonizes human skin and mucosa without causing severe problems, but if the bacteria enter the body, illnesses that range from mild to life-threatening can develop, including skin and wound infections, infected eczema, abscess infections, heart valve infections or endocarditis, pneumonia, and bloodstream infections or bacteremia. Some S. aureus are resistant to methicillin and other β-lactam antibiotics-methicillin-resistant S. aureus (MRSA)—and require alternative types of antibiotics to treat them.

There is a need for antiviral and antimicrobial materials that exhibit broad-spectrum antiviral and/or antimicrobial activity and that can used in a variety of environments (e.g., homes, healthcare facilities, schools), surfaces (e.g., wood, stainless steel, marble, glass, and textiles), and applications (e.g., food packaging, water or air filters). The materials can have high kill rate, be viable for weeks, and be non-toxic. This disclosure fulfills these needs and provides further advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure features a method of inhibiting microorganisms on a surface, including applying to the surface a polymer including a repeating unit, the repeating unit including:
(i) a benzimidazolium-containing moiety of Formula (I):

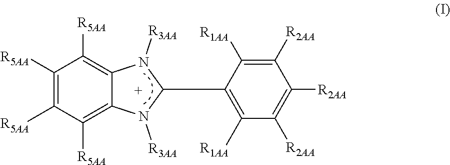

wherein:
R_{1AA} is independently selected from the group consisting of H, methyl, trifluoromethyl, alkyl, perfluoroalkyl, heteroalkyl, alkoxy, perfluoroalkoxy, halo, aryl, heteroaryl group and a polymer;
R_{2AA} is independently selected from the group consisting of hydrogen, any group, and a polymer;
R_{3AA} is independently selected from the group consisting of methyl, trifluoromethyl, alkyl, perfluoroalkyl, heteroalkyl, aryl, aralkyl, a polymer, and no group; and
R_{5AA} is independently selected from the group consisting of hydrogen, any group, and a polymer;
wherein at least one of R_{1AA}, R_{2AA}, R_{3AA}, and R_{5AA}, is a polymer;
(ii) an imidazolium-containing moiety having Formula (II):

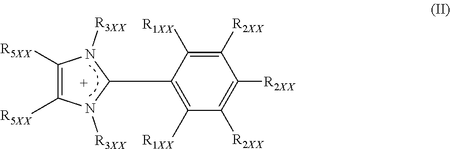

wherein:
R_{1XX} is independently selected from the group consisting of H, methyl, trifluoromethyl, alkyl, perfluoroalkyl, heteroalkyl, alkoxy, perfluoroalkoxy, halo, aryl, heteroaryl group and a polymer;
R_{2XX} is independently selected from the group consisting of hydrogen, any group, and a polymer;
R_{3XX} is independently selected from the group consisting of methyl, trifluoromethyl, alkyl, perfluoroalkyl, heteroalkyl, aryl, aralkyl, a polymer, and no group; and
R_{5XX} is independently selected from the group consisting of hydrogen, any group, and a polymer;
wherein at least one of R_{1XX}, R_{2XX}, R_{3XX}, and R_{5XX}, is a polymer, or
any combination thereof.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1D are a series of graphs showing antimicrobial action of embodiments of polymer coatings of the present disclosure against viruses and bacteria.

FIG. 2 is a scanning electron micrograph of an embodiment of a polymeric coating of the present disclosure. Unlike nano-silver, the polymeric coatings of the present disclosure provide a smooth surface that enhances antimicrobial activity. Cryo-fractured SEM on high surface area carbon (>3000 m²/g) shows infiltration depths in the range of 0.3-0.6 μm (300-600 nm). An area loading equivalent to about 1 μm can provide a cohesive coating, while <1 μm coatings can be readily formed on less porous surfaces.

FIG. 3A is a graph of % transmittance as a function of wavelength for a representative polymeric coating of the present disclosure and FIG. 3B is a thermogravimetric analysis (TGA) scan of the same.

FIG. 4 is a table of embodiments of polymeric coatings of the present disclosure. Water uptake can vary by counterion. The chloride+low functionality variant is very hydrophobic, the chloride or acetate+high functionality variants are hydrophilic, and the nitrate+high functionality variant is an in-between. The hydrophilicity/hydrophobicity can provide differentiation in antibacterial and/or antiviral properties. For example, changes in hydrophilicity can provide alterations in interactions with bacterial cell walls, viral envelopes, and influence polymer mobility.

FIG. 5 is a schematic representation of an antimicrobial and/or antiviral tests. The ISO/JIS standards chosen are considered “gold standard” tests, and are designed to test the antimicrobial properties of non-porous surfaces. The test involves six data points per sample (triplicate+‘plated’ twice), and an additional blank/non-coated glass slide control. The antiviral tests performed included soiling (5% blood serum in viral inoculation solution) and included testing for cytotoxicity as a potential confounding variable (all negative).

FIG. 6 is a table of parameters for antimicrobial and/or antiviral tests of embodiments of polymeric coatings of the present disclosure. S. aureus (Gram+) and E. coli (Gram −) were representative in antimicrobial testing for G+ and G-bacteria. Human Coronavirus (229E) was used as a model of enveloped viruses, such as coronaviruses (e.g., SARS-COV-2). Feline Calicivirus (F-9) was used as a model for non-enveloped viruses, such as respiratory viruses (e.g., the calicivirus family). Ultra-thin films ˜0.75-1.5 μm were tested.

FIG. 7 shows tables of antimicrobial testing results for embodiments of polymeric coatings of the present disclosure.

FIG. 8 is a table of kinetic analysis of antimicrobial testing results for embodiments of polymeric coatings of the present disclosure. Unprecedented activity for a surface modification was observed-half-lives for Human Coronavirus (229E), G+, and G-bacteria were demonstrated as little as <2.1 minutes-a 4-5 log reduction to beyond the detectable limit within 30 minutes. All variants passed JIS/ISO protocols for antibacterial activity (log 10 reduction >2 in 24 h). Acetate and 1.5 μm “thick” chloride films had better activity-ultra-thin coatings can increase hydrophobicity.

FIG. 9 is a schematic representation of a drip flow reactor.

FIG. 10 is a photograph of a drip flow reactor set-up in continuous flow operation.

FIG. 11 is a schematic side view of a drip flow reactor used for calculating the reactor angle.

### DETAILED DESCRIPTION

The present disclosure features a method of inhibiting microorganisms on a surface, including applying to the surface a polymer including a repeating unit, the repeating unit including:
(i) a benzimidazolium-containing moiety of Formula (I):

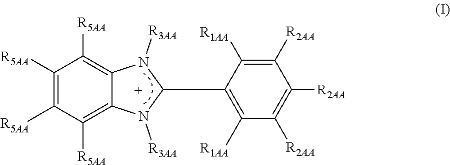

wherein:
R_{1AA} is independently selected from the group consisting of H, methyl, trifluoromethyl, alkyl, perfluoroalkyl, heteroalkyl, alkoxy, perfluoroalkoxy, halo, aryl, heteroaryl group and a polymer;
R_{2AA} is independently selected from the group consisting of hydrogen, any group, and a polymer;
R_{3AA} is independently selected from the group consisting of methyl, trifluoromethyl, alkyl, perfluoroalkyl, heteroalkyl, aryl, aralkyl, a polymer, and no group; and
R_{5AA} is independently selected from the group consisting of hydrogen, any group, and a polymer;
wherein at least one of R_{1AA}, R_{2AA}, R_{3AA}, and R_{5AA}, is a polymer;
(ii) an imidazolium-containing moiety having Formula (II):

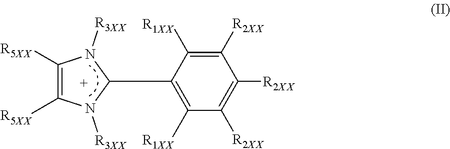

wherein:
R_{1XX} is independently selected from the group consisting of H, methyl, trifluoromethyl, alkyl, perfluoroalkyl, heteroalkyl, alkoxy, perfluoroalkoxy, halo, aryl, heteroaryl group and a polymer;
R_{2XX} is independently selected from the group consisting of hydrogen, any group, and a polymer;
R_{3XX} is independently selected from the group consisting of methyl, trifluoromethyl, alkyl, perfluoroalkyl, heteroalkyl, aryl, aralkyl, a polymer, and no group; and
R_{5XX} is independently selected from the group consisting of hydrogen, any group, and a polymer;
wherein at least one of R_{1XX}, R_{2XX}, R_{3XX}, and R_{5XX}, is a polymer,
or any combination thereof.

Examples of polymers are described, for example, in PCT publication nos. WO2013/149328, WO2015/157848, WO2018/026743, and WO2018/023097, each of which is incorporated herein in its entirety.

The polymer can effectively render the coated surface bactericidal, virucidal, and/or germicidal, such that microorganisms (e.g., fungi, viruses, and/or microbes [e.g., bacteria or single-cell organisms]) on the surface are killed and/or inhibited on the coated surface. In some embodiments, the polymer can inhibit adhesion of microorganisms on the surface.

The polymers of the present disclosure can form a clear, transparent, durable, ultra-thin film on any surface with a variety of solvents, such as non-toxic solvents. The polymeric coatings can provide unprecedented antimicrobial activity. In some embodiments, up to ≥99.995% reduction of enveloped viruses, G-bacteria (E. coli), and G+ bacteria (S. aureus) can be seen in ≤30 minutes. Half-lives of viruses and bacteria can be on the order less than 10 minutes (e.g., less than 5 minutes, less than 3 minutes). Unlike silver nanoparticles and nanostructured surfaces, the universally applicable, cost-effective, polymeric coatings of the present disclosure do not lose effectiveness under mechanical stress (e.g., through wash/disinfectant cycles). The coated surfaces can have increased smoothness, which can enhance the antimicrobial properties of the surface. Furthermore, the coatings can be non-toxic to humans and the environment; and can be chemically (e.g., to soiling, fouling, UV, disinfectant, and bleach) and mechanically stable.

In some embodiments, when the polymer-coated surface is exposed to the microorganism, the number of microorganisms on the surface can be reduced by greater than 99% after a duration of 24 hours (e.g., less than 24 hours, less than 20 hours, less than 12 hours, less than 6 hours, less than 3 hours, less than 1 hour, less than 30 minutes, less than 20 minutes, less than 10 minutes, or less than 5 minutes). The microorganism can include a fungus (i.e., one or more species of a fungus), a bacterium (i.e., one or more species of a bacterium), a virus (i.e., one or more species of a virus), or any combination thereof.

In some embodiments, when the polymer-coated surface is exposed to a fungus, the number of the fungus on the surface can be reduced by greater than 99% after a duration of 24 hours (e.g., less than 24 hours, less than 20 hours, less than 12 hours, less than 6 hours, less than 3 hours, less than 1 hour, less than 30 minutes, less than 20 minutes, less than 10 minutes, or less than 5 minutes).

In some embodiments, when the polymer-coated surface is exposed to a bacterium, the number of the bacterium is reduced by greater than 99% after a duration of 24 hours. The bacterium can be a gram-positive bacterium, and/or a gram-negative bacterium. The bacterium can be pathogenic.

In some embodiments, when the polymer-coated surface is exposed to a virus, the number of the virus is reduced by greater than 99% after a duration of 24 hours (e.g., less than 24 hours, less than 20 hours, less than 12 hours, less than 6 hours, less than 3 hours, less than 1 hour, less than 30 minutes, less than 20 minutes, less than 10 minutes, or less than 5 minutes).

### Definitions

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further intended that the compounds of the disclosure are stable. As used herein “stable” refers to a compound that is sufficiently robust to survive isolation to a useful degree of purity from a reaction mixture.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

“Optionally substituted” groups can refer to, for example, functional groups that may be substituted or unsubstituted by additional functional groups. For example, when a group is unsubstituted, it can be referred to as the group name, for example alkyl or aryl. When a group is substituted with additional functional groups, it may more generically be referred to as substituted alkyl or substituted aryl.

As used herein, the term “substituted” or “substitution” refers to the replacing of a hydrogen atom with a substituent other than H. For example, an “N-substituted piperidin-4-yl” refers to replacement of the H atom from the NH of the piperidinyl with a non-hydrogen substituent such as, for example, alkyl.

As used herein, the term “alkyl” refers to a straight or branched hydrocarbon groups having the indicated number of carbon atoms. Representative alkyl groups include methyl, ethyl, propyl (e.g., n-propyl, isopropyl), butyl (e.g., n-butyl, sec-butyl, and tert-butyl), pentyl (e.g., n-pentyl, tert-pentyl, neopentyl, isopentyl, pentan-2-yl, pentan-3-yl), and hexyl (e.g., n-pentyl and isomers) groups.

As used herein, the term “alkylene” refers to a linking alkyl group.

As used herein, the term “perfluoroalkyl” refers to straight or branched fluorocarbon chains. Representative alkyl groups include trifluoromethyl, pentafluoroethyl, etc.

As used herein, the term “perfluoroalkylene” refers to a linking perfluoroalkyl group.

As used herein, the term “heteroalkyl” refers to a straight or branched chain alkyl groups having the indicated number of carbon atoms and where one or more of the carbon atoms is replaced with a heteroatom selected from O, N, or S.

As used herein, the term “heteroalkylene” refers to a linking heteroalkyl group.

As used herein, the term “alkoxy” refers to an alkyl or cycloalkyl group as described herein bonded to an oxygen atom. Representative alkoxy groups include methoxy, ethoxy, propoxy, and isopropoxy groups.

As used herein, the term “perfluoroalkoxy” refers to a perfluoroalkyl or cyclic perfluoroalkyl group as described herein bonded to an oxygen atom. Representative perfluoroalkoxy groups include trifluoromethoxy, pentafluoroethoxy, etc.

As used herein, the term “aryl” refers to an aromatic hydrocarbon group having 6 to 10 carbon atoms. Representative aryl groups include phenyl groups. In some embodiments, the term “aryl” includes monocyclic or polycyclic (e.g., having 2, 3, or 4 fused rings) aromatic hydrocarbons such as, for example, phenyl, naphthyl, anthracenyl, phenanthrenyl, indanyl, and indenyl.

As used herein, the term “arylene” refers to a linking aryl group.

As used herein, the term “aralkyl” refers to an alkyl or cycloalkyl group as defined herein with an aryl group as defined herein substituted for one of the alkyl hydrogen atoms. A representative aralkyl group is a benzyl group.

As used herein, the term “aralkylene” refers to a linking aralkyl group.

As used herein, the term “heteroaryl” refers to a 5- to 10-membered aromatic monocyclic or bicyclic ring containing 1-4 heteroatoms selected from O, S, and N. Representative 5- or 6-membered aromatic monocyclic ring groups include pyridine, pyrimidine, pyridazine, furan, thiophene, thiazole, oxazole, and isooxazole. Representative 9- or 10-membered aromatic bicyclic ring groups include benzofuran, benzothiophene, indole, pyranopyrrole, benzopyran, quionoline, benzocyclohexyl, and naphthyridine.

As used herein, the term “heteroarylene” refers to a linking heteroaryl group.

As used herein, the term “halogen” or “halo” refers to fluoro, chloro, bromo, and iodo groups.

As used herein, the term “bulky group” refers to a group providing steric bulk by having a size at least as large as a methyl group.

As used herein, the term “copolymer” refers to a polymer that is the result of polymerization of two or more different monomers. The number and the nature of each constitutional unit can be separately controlled in a copolymer. The constitutional units can be disposed in a purely random, an alternating random, a regular alternating, a regular block, or a random block configuration unless expressly stated to be otherwise. A purely random configuration can, for example, be: x-x-y-z-x-y-y-z-y-z-z-z . . . or y-z-x-y-z-y-z-x-x . . . . An alternating random configuration can be: x-y-x-z-y-x-y-z-y-x-z . . . , and a regular alternating configuration can be: x-y-z-x-y-z-x-y-z . . . . A regular block configuration (i.e., a block copolymer) has the following general configuration: . . . x-x-x-y-y-y-z-z-Z-x-x-x . . . , while a random block configuration has the general configuration: . . . x-x-x-z-z-x-x-y-y-y-y-z-z-z-x-x-z-z-z- . . . .

As used herein, the term “random copolymer” is a copolymer having an uncontrolled mixture of two or more constitutional units. The distribution of the constitutional units throughout a polymer backbone (or main chain) can be a statistical distribution, or approach a statistical distribution, of the constitutional units. In some embodiments, the distribution of one or more of the constitutional units is favored.

As used herein, the term “constitutional unit” of a polymer refers to an atom or group of atoms in a polymer, comprising a part of the chain together with its pendant atoms or groups of atoms, if any. The constitutional unit can refer to a repeat unit. The constitutional unit can also refer to an end group on a polymer chain. For example, the constitutional unit of polyethylene glycol can be —CH₂CH₂O— corresponding to a repeat unit, or —CH₂CH₂OH corresponding to an end group.

As used herein, the term “repeat unit” corresponds to the smallest constitutional unit, the repetition of which constitutes a regular macromolecule (or oligomer molecule or block).

As used herein, the term “end group” refers to a constitutional unit with only one attachment to a polymer chain, located at the end of a polymer. For example, the end group can be derived from a monomer unit at the end of the polymer, once the monomer unit has been polymerized. As another example, the end group can be a part of a chain transfer agent or initiating agent that was used to synthesize the polymer.

As used herein, the term “terminus” of a polymer refers to a constitutional unit of the polymer that is positioned at the end of a polymer backbone.

As used herein, the term “cationic” refers to a moiety that is positively charged, or ionizable to a positively charged moiety under physiological conditions. Examples of cationic moieties include, for example, amino, ammonium, pyridinium, imino, sulfonium, quaternary phosphonium groups, etc.

As used herein, the term “anionic” refers to a functional group that is negatively charged, or ionizable to a negatively charged moiety under physiological conditions. Examples of anionic groups include carboxylate, sulfate, sulfonate, phosphate, etc.

As used herein, the term “applying” refers to any suitable technique used to transfer a polymer composition to a surface. For example, techniques for applying can be, but are not limited to, brushing, rolling, spraying, wiping, mopping, pouring, painting, absorbing, adsorbing, imbibing, soaking, saturating, permeating, immersing, and a combination of these methods.

As used herein, the term “bactericidal, virucidal, and/or germicidal” refers to reducing (e.g., eliminating, killing, or preventing and/or inhibiting growth) the presence of microorganisms, such as bacteria, viruses, and/or germs (including a fungus, such as Aspergillas brasliensis) to any suitable degree. As used herein, the term “any suitable degree” refers to 50% reduction or more, including 60% reduction or more, 70% reduction or more, 80% reduction or more, 90% reduction or more, 92% reduction or more, 94% reduction or more, 95% reduction or more, 97% reduction or more, 98% reduction or more, 99% reduction or more, or 99.5% reduction or more.

As used herein, the term “microorganism” includes a germ (e.g., a fungus), a single-celled organism (e.g., a bacterium, an archaeon), and/or a virus.

As used herein, a “medical device” includes any device having surfaces that contact tissue, blood, or other bodily fluids in the course of their use or operation, which are found on or are subsequently used within a mammal (e.g., a human). Medical devices include, for example, extracorporeal devices for use in surgery, such as blood oxygenators, blood pumps, blood storage bags, blood collection tubes, blood filters including filtration media, dialysis membranes, tubing used to carry blood and the like which contact blood which is then returned to the patient or mammal. Medical devices also include endoprostheses implanted in a mammal (e.g., a human), such as vascular grafts, stents, pacemaker leads, surgical prosthetic conduits, heart valves, and the like, that are implanted in blood vessels or the heart. Medical devices also include devices for temporary intravascular use such as catheters, guide wires, amniocentesis and biopsy needles, cannulae, drainage tubes, shunts, sensors, transducers, probes and the like which are placed into the blood vessels, the heart, organs or tissues for purposes of monitoring or repair or treatment. Medical devices also include prostheses such as artificial joints such as hips or knees as well as artificial hearts. In addition, medical devices include penile implants, condoms, tampons, sanitary napkins, ocular lenses, sling materials, sutures, hemostats used in surgery, surgical mesh, transdermal patches, and wound dressings/bandages.

As used herein, a “diagnostic equipment” includes any device or tool used to diagnose or monitor a medical condition. Examples include an ultrasound, magnetic resonance imaging (MRI) machine, positron emission tomography (PET) scanner, computed tomography (CT) scanner, ventilator, heart-lung machine, extracorporeal membrane oxygenation (ECMO) machine, dialysis machine, blood pressure monitor, otoscope, ophthalmoscope, stethoscope, sphygmomanometer, blood pressure cuff, electrocardiogra thermometer, defibrillator, speculum, sigmoidoscope, and anoscope.

As used herein, a “surgical instrument” includes any tool or device used for performing surgery or an operation. Examples include a scalpel, lancet, trocar, hemostat, grasper, forceps, clamp, retractor, distractor, positioner, tracheotome, dilator, stapler, irrigation needle, injection needle, drill, scope, endoscope, probe, ruler, and caliper.

As used herein, a “safety gear” includes devices used to protect a person, animal, or object. Examples of “safety gear” are a mask, face shield, visor, goggles, glasses, gloves, shoe covers, foot guard, leg guard, belt, smock, apron, coat, vest, raingear, hat, helmet, chin strap, hairnet, shower cap, hearing protection (ear plugs, ear muffins, hearing bands), respirator, gas mask, supplied air hood, collar, leash, and first aid kit.

As used herein, a “fabric” includes any type of suitable fabric, such as bedding, curtains, towels, table coverings, protective sheeting, and dish cloths.

As used herein, an “apparel item” includes an item of clothing, footwear, or other item someone would wear on his/her person. Examples include a uniform, coat, shirt, pants, waders, scrubs, socks, shoe or boot liner, an insole, gloves, hats, shoes, boots, and sandals.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the FIGURES should not be viewed as limiting. It should be understood that other embodiments may include more or less of each element shown in a given FIGURE. Further, some of the illustrated elements may be combined or omitted. Yet further, an example embodiment may include elements that are not illustrated in the FIGURES. As used herein, with respect to measurements, “about” means±5%. As used herein, a recited ranges includes the end points, such that from 0.5 mole percent to 99.5 mole percent includes both 0.5 mole percent and 99.5 mole percent.

### Articles

The polymer can be in the form of a coating on a surface or a membrane that can be applied to a surface. In some embodiments, the polymer (e.g., the polymer in the coating or the membrane) is in the form of fibers, such as spun fibers, electrospun fibers, extruded fibers, nanofibers, or any combination thereof. In some embodiments the polymer can be coated onto the surface by any method, such as solution cast coating. In certain embodiments, the polymer can be in the form of a microporous film, nanoporous film, non-woven sheet, woven sheet, mat, fabric, or any combination thereof.

The polymer can be applied as a pure polymer, or as part of a blend with other materials. For example, the polymer can be in a blend with one or more other polymers. In certain embodiments, the polymer is a graft copolymer, an interpenetrating network, an aerogel, and/or a hydrogel.

The surface to which the polymer can be applied can include a surface of a medical device or a portion thereof. For example, the medical device can be an urinary catheter, a percutaneous catheter, a central venous catheter, a vascular access device, a heart valve, a stent, a vascular prosthesis, a skeletal joint, a dental filling, a dental implant, an oxygen transport membrane, a suture, an intravenous delivery site, a drug delivery catheter, a drain, a gastric feeding tube, a tracheotomy tube, a contact lens, an intraocular lens, an orthopedic implant, a neuro-stimulation lead, a pacemaker lead, a blood bag, an air filter, and/or a drug diffusion matrix (e.g., membranes, films, rods, beads, or any combination thereof). In certain embodiments, the medical device is a contact lens, an intraocular lens, or an air filter.

In some embodiments, the polymer is incorporated into a water treatment apparatus or a medical device. In some embodiments, the polymer is the form of a separation membrane in a water treatment apparatus. In some embodiments, the polymer can be coated onto, or otherwise incorporated into a medical device (e.g., a portion of a medical device, a surface of a medical device, a portion of a surface of a medical device), where the medical device can include urinary catheters, percutaneous catheters, central venous catheters, vascular access devices, heart valves, stents, vascular prostheses, skeletal joints, dental fillings, dental implants, oxygen transport membranes, sutures, intravenous delivery sites, drug delivery catheters, drains, gastric feeding tubes, tracheotomy tubes, contact lenses, intraocular lenses, orthopedic implants, neuro-stimulation leads, pace maker leads, blood bags, air filters, and/or drug diffusion matrices including membranes, films, rods, or beads. In certain embodiments, the medical device is a contact lens, an intraocular lens, and/or an air filter. For example, the medical device can be an air filter.

The polymers of the present disclosure can be applied to, or incorporated into, various articles where antiviral and/or antimicrobial properties are advantageous. For example, the articles can be found in the food or beverage industry (such as tanks, conveyors, floors, drains, coolers, freezers, refrigerators, equipment surfaces, walls, valves, belts, pipes, drains, joints, crevasses, combinations thereof, and the like); building surfaces (such as walls, wood frames, floors, windows), kitchens (sinks, drains, counter-tops, refrigerators, cutting boards), bathrooms (showers, toilets, drains, pipes, bath-tubs), (decks, wood, siding and other home exteriors, asphalt shingle roofing, patio or stone areas (especially for algae treatment); boats and boating equipment surfaces; airplanes and airplane equipment surfaces; garbage disposals, garbage cans and dumpsters or other trash removal equipment and surfaces; non-food-industry related pipes and drains; surfaces in hospital, surgery or out-patient centers or veterinary surfaces (such as walls, floors, beds, equipment, clothing worn in hospital/veterinary or other healthcare settings, including scrubs, shoes, and other hospital or veterinary surfaces) first-responder or other emergency services equipment and clothing; lumber-mill equipment, surfaces and wood products; bathroom surfaces such as sinks, showers, counters, and toilets; clothes and shoes; toys; school and gymnasium equipment, walls, floors, windows and other surfaces; kitchen surfaces such as sinks, counters, appliances; wooden or composite decks, pool, hot tub and spa surfaces; carpet; paper; leather; animal carcasses, fur and hides.

The polymers of the present disclosure can be coated onto, or incorporated into, fibrous substrates and include fibers, yarns, fabrics, textiles, nonwovens, carpets, leather, or paper. The fibrous substrates are made with natural fibers such as wool, cotton, jute, sisal, sea grass, paper, coir and cellulose, or mixtures thereof; or are made with synthetic fibers such as polyamides, polyesters, polyolefins, polyaramids, acrylics and blends thereof; or blends of at least one natural fiber and at least one synthetic fiber. By “fabrics” is meant natural or synthetic fabrics, or blends thereof, composed of fibers such as cotton, rayon, silk, wool, polyester, polypropylene, polyolefins, nylon, and aramids such as “NOMEX®” and “KEVLAR®.” By “fabric blends” is meant fabric made of two or more types of fibers. Typically these blends are a combination of at least one natural fiber and at least one synthetic fiber, but also can be a blend of two or more natural fibers or of two or more synthetic fibers. Nonwoven substrates include, for example, spunlaced nonwovens, such as SONTARA available from E. I. du Pont de Nemours and Company (Wilmington, Del., USA), and laminated nonwovens, such as spunbonded-meltblown-spunbonded nonwovens.

Examples of surface materials on which the polymers can be coated, or incorporated into, include metals (e.g., steel, stainless steel, chrome, titanium, iron, copper, brass, aluminum, and alloys thereof), minerals (e.g., concrete), other polymers and plastics (e.g., polyolefins, such as polyethylene, polypropylene, polystyrene, poly(meth) acrylate, polyacrylonitrile, polybutadiene, poly(acrylonitrile, butadiene, styrene), poly(acrylonitrile, butadiene), acrylonitrile butadiene; polyesters such as polyethylene terephthalate; and polyamides such as nylon). Additional surfaces include brick, tile, ceramic, porcelain, wood, vinyl, and linoleum.

The polymer of the present disclosure can be a component (e.g., a surface, a bulk, or a portion) of a larger structure. For example, the polymer can be part of a substrate, such as a medical device, diagnostic equipment, implant, glove, mask, curtain, mattress, sheets, blankets, gauze, dressing, tissue, surgical drape, tubing, surgical instrument, safety gear, fabric, apparel item, floor, handles, wall, sink, shower or tub, toilet, furniture, wall switch, toy, athletic equipment, playground equipment, shopping cart, countertop, appliance, railing, door, air filter, pipe, utensil, dish, cup, container, object display container, food, food display container, food package, food processing equipment, food handling equipment, food transportation equipment, food vending equipment, food storage equipment, food packaging equipment, plant, phone, cell phone, remote control, computer, mouse, keyboard, touch screen, leather, cosmetic, cosmetic making equipment, cosmetics storage equipment, cosmetics packaging equipment, personal care item, personal care item making equipment, personal care storage equipment, personal care packaging equipment, animal care item, animal care item making equipment, veterinary equipment, powder, cream, gel, salve, eye care item, eye care item making equipment, contact lens, glasses, eye care storage equipment, contact lens case, jewelry, jewelry making equipment, jewelry storage equipment, animal housing, farming equipment, animal food handling equipment, animal food storage space, animal food storage equipment, animal food container, air vehicle, land vehicle, air processing equipment, air filter, water vehicle, water storage space, water storage equipment, water processing equipment, water storage container, water filter, pharmaceuticals display container, pharmaceuticals package, pharmaceuticals processing equipment, pharmaceuticals handling equipment, pharmaceuticals transportation equipment, pharmaceuticals vending equipment, pharmaceuticals, pharmaceuticals storage equipment, and/or pharmaceuticals packaging equipment.

The polymer can be incorporated into a part of or coated onto a toy or athletic equipment, including exercise equipment, playground equipment, or a pool.

The “animal care item” and “veterinary equipment” can be any product used in a setting that includes animals, such as a house, boarding house, or veterinary hospital. Of course, veterinary equipment can be used at a location outside of a hospital setting. Animals are any animals that are typically considered pets, non-pets, boarded, treated by a veterinarian, and animals in the wild. Examples include a dog, cat, reptile, bird, rabbit, ferret, guinea pig, hamster, rat, mouse, fish, turtle, horse, goat, cattle, and pigs. Suitable animal care items include the personal care items described herein, toys, bed, crate, kennel, carrier, bowl, dish, leash, collar, litterbox, and grooming items (e.g., clippers, scissors, a brush, comb, dematting tool, and deshedding tool). Suitable veterinary equipment includes any of the medical devices and surgical instruments described herein and other equipment, such as a table, tub, stretcher, sink, scale, cage, carrier, and leash.

The “animal housing” can be any suitable housing, such as a coop, stable, shelter, grab bag shelter, hutch, barn, shed, pen, nestbox, feeder, stanchion, cage, carrier, or bed.

The “farming equipment” is any device used in an agricultural setting, including a farm or ranch, particularly a farm or ranch that houses animals, processes animals, or both. Animal livestock that can be housed or processed as described herein and include, e.g., horses, cattle, bison, and small animals such as poultry (e.g., chickens, quails, turkeys, geese, ducks, pigeons, doves, pheasants, swan, ostrich, guineafowl, Indian peafowl, emu), pigs, sheep, goats, alpacas, llamas, deer, donkeys, rabbits, and fish. Examples of farming equipment include as a wagon, trailer, cart, barn, shed, fencing, sprinkler, shovel, scraper, halter, rope, restraining equipment, feeder, waterer, trough, water filter, water processing equipment, stock tank, fountain, bucket, pail, hay rack, scale, poultry flooring, egg handling equipment, a barn curtain, tractor, seeder, planter, plow, rotator, tiller, spreader, sprayer, agitator, sorter, baler, harvester, cotton picker, thresher, mower, backhoe loader, squeeze chute, hydraulic chute, head chute, head gate, crowding tub, corral tub, alley, calving pen, calf table, and milking machine.

The article can be part of a vehicle, such as an air vehicle, land vehicle, or water vehicle. Suitable vehicles include a car, van, truck, bus, ambulance, recreational vehicle, camper, motorcycle, scooter, bicycle, wheelchair, train, streetcar, ship, boat, canoe, submarine, an unmanned underwater vehicle (UUV), a personal watercraft, airplane, jet, helicopter, unmanned autonomous vehicle (UAV), and hot air balloon

### Microorganisms

In some embodiments, the microorganism that is inhibited or killed by the polymeric coatings of the present disclosure is a gram-positive bacterium. In certain embodiments, the microorganism is a gram-negative bacterium. The bacterium can be pathogenic. For example, the pathogenic bacterium can be selected from Escherichia coli, Staphylococcus aureus, Campylobacter spp., Legionella, Salmonella spp., Shigella spp., Tsukamurella, Leptospires, Mycobacterium, Pseudomonas aeruginosa, Aeromonas spp., Vibrio spp., Yersinia, Bacillus spp., Enterobacter sakazakii, Burkholderia pseudomallei, Acinetobacter spp., Helicobacter pylori, Klebsiella spp., clostridium spp., and Streptococci. The term “pathogen” and “pathogenic” are used herein interchangeably and refer to bacteria, fungi, viruses, and other microorganisms capable of exerting pathogenic effects in multicellular organisms. Thus, the use of the term “pathogen” contemplates microorganisms capable of causing disease in plants, mammals, including humans.

Further exemplary gram-positive bacteria which are inhibited or killed by the polymeric coating include, but are not limited to, Mycobacterium tuberculosis, M. bovis, M. typhimurium, M. bovis strain BCG, BCG substrains, M. avium, M. intracellulare, M. africanum, M. kansasii, M. marinum, M. ulcerans, M. avium subspecies paratuberculosis, Staphylococcus aureus, S. epidermidis, S. equi, Streptococcus pyogenes, S. agalactiae, Listeria monocytogenes, L. ivanovii, Bacillus anthracis, B. subtilis, Nocardia asteroides, and other Nocardia species, Streptococcus viridans group, Peptococcus species, Peptostreptococcus species, Actinomyces israelii and other Actinomyces species, Propionibacterium acnes, and Enterococcus species. Gram negative bacteria which are inhibited or killed by the polymeric coating include, but are not limited to, Clostridium tetani, C. perfringens, C. botulinum, other Clostridiumspecies, Pseudomonas aeruginosa, other Pseudomonas species, Campylobacterspecies, Vibrio cholerae, Ehrlichia species, Actinobacillus pleuropneumoniae Pasteurella haemolytica, P. multocida, other Pasteurella species, Legionella pneumophila, other Legionella species, Salmonella typhi, other Salmonella species, Shigella species Brucella abortus, other Brucella species, Chlamydia trachomatis, C. psittaci, Coxiella burnetti, Escherichia coli, Neiserria meningitidis, N. gonorrhea, Haemophilus influenzae, H. ducreyi, other Haemophilus species, Yersinia pestis, Y. enterolitica, other Yersinia species, Escherichia coli, E. hirae and other Escherichiaspecies, as well as other Enterobacteriacae, Brucella abortus and other Brucellaspecies, Burkholderia cepacia, B. pseudomallei, Francisella tularensis, Bacteroides fragilis, Fusobacterium nucleatum, Provetella species, Cowdria ruminantium, Klebsiella species, and Proteus species.

In some embodiments, the microorganism that is inhibited or killed by the polymeric coatings of the present disclosure is a virus. The virus can be selected from alphacoronaviruses, betacoronaviruses (e.g., SARS-COV-2), deltacoronaviruses, gammacoronaviruses, and toroviruses; influenza virus A, influenza virus B, influenza virus C, influenza virus D, hepatitis A virus, hepatitis B virus, simplex viruses, cytomegaloviruses, mamastroviruses, mastadenoviruses, poxviruses, hepadnaviruses, asfarviridae, flaviviruses, alphaviruses, togaviruses, paramyxoviruses, rhabdovirus, bunyaviruses, filoviruses, retroviridae, coxsackieviruses (enveloped and non-enveloped), rotaviruses, polioviruses, calciviruses, orthopoxviruses, polyomaviruses, vesiculoviruses, varicelloviruses, phleboviruses, alphatorqueviruses, noroviruses, rubulaviruses, spumaviruses, rubulaviruses, hantaviruses, sapoviruses, saliviruses, rubiviruses, rosaviruses, lyssaviruses, enteroviruses, arenaviruses, orthobunyaviruses, parapoxviruses, henipaviruses, molluscipoxviruses, polyomaviruses, cardioviruses, morbilviruses, marburgviruses, deltaretroviruses, orthopneumoviruses, erythroviruses, respiroviruses, alphapapillomavirus, mupapillomaviruses, rhadinoviruses, roseoloviruses, deltaviruses, hepeviruses, hepaciviruses, orthohepadnaviruses, henipaviruses, pegiviruses, lymphocryptoviruses, ebolaviruses, nairoviruses, thogotoviruses, cosaviruses, seadornaviruses, kobuviruses, dependoviruses, or any combination thereof. In certain embodiments, the virus is a coronavirus, such as a SARS-COV or SARS-COV2 virus.

In some embodiments, the microorganism that is inhibited or killed by the polymeric coatings of the present disclosure includes a fungus. For example, the fungus can be Candida spp., Cryptococcus spp., Blastomyces spp., Coccidioides spp., Aspergillus spp., Emmonsia spp., Chrysosporium spp., Fonsecaea spp., Trychophyon spp., Histoplasma spp., Ajellomyces spp., Pneumocytis spp., Microsporum spp., Magnaporthe spp., Fusarium spp., Sporothrix spp., Cyberlindnera spp., Mucormycetes spp., Epidermophyton spp., Trichosporon spp., Paracoccidioides spp., Talaromyces spp., Uncinocarpus reesii, and Aphanoascus spp. In some embodiments, the fungus is Candida spp. or Cryptococcus spp. Other exemplary fungi that are inhibited or killed by the polymeric coatings of the present disclosure include Alternaria alternata, Aspergillus niger, Aureobasidium pullulans, Cladosporium cladosporioides, Drechslera australiensis, Gliomastix cerealis, Monilia grisea, Penicillium commune, Phoma fimeti, Pithomyces chartarum, Scolecobasidium humicola, or any combination thereof.

### Polymers

In one aspect, a polymer is provided. The polymer comprises one or more repeating units (“monomer units”). In one embodiment, at least one of the monomer units comprises one or more benzimidazolium-containing moieties M1 having Formula (I):

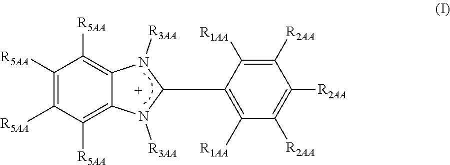

wherein:
R_{1AA} is independently selected from the group consisting of H, methyl, trifluoromethyl, alkyl, perfluoroalkyl, heteroalkyl, alkoxy, perfluoroalkoxy, halo, aryl, heteroaryl group and a polymer;
R_{2AA} is independently selected from the group consisting of hydrogen, any group, and a polymer;
R_{3AA} is independently selected from the group consisting of methyl, trifluoromethyl, alkyl, perfluoroalkyl, heteroalkyl, aryl, aralkyl, a polymer, and no group; and
R_{5AA} is independently selected from the group consisting of hydrogen, any group, and a polymer;
wherein at least one of R_{1AA}, R_{2AA}, R_{3AA}, and R_{5AA}, is a polymer;

These “R-group” definitions are consistent for all Formulas, Figures, and Schemes throughout the disclosure. At each instance, every R-group is independently selected.

In another aspect, another polymer is provided. The polymer comprises one or more repeating units. In one embodiment, at least one of the repeating units comprises one or more imidazolium-containing moieties M2 having Formula (II):

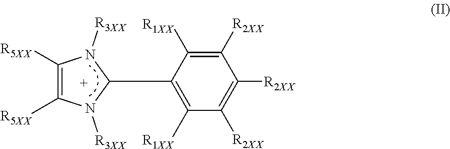

wherein:
R_{1XX} is independently selected from the group consisting of H, methyl, trifluoromethyl, alkyl, perfluoroalkyl, heteroalkyl, alkoxy, perfluoroalkoxy, halo, aryl, heteroaryl group and a polymer;
R_{2XX} is independently selected from the group consisting of hydrogen, any group, and a polymer;
R_{3XX} is independently selected from the group consisting of methyl, trifluoromethyl, alkyl, perfluoroalkyl, heteroalkyl, aryl, aralkyl, a polymer, and no group; and
R_{5XX} is independently selected from the group consisting of hydrogen, any group, and a polymer;
wherein at least one of R_{1XX}, R_{2XX}, R_{3XX}, and R_{5XX}, is a polymer. At each instance, every R-group is independently selected.

As used herein, the term moiety is used to refer to one or both of the moieties of Formulas (I) and (II). Together, they may be referred to as moieties. They may also be referred to as the imidazolium or benzimidazolium moieties.

The steric crowding of the provided moieties results from the interaction of the R₃ imidazolium or benzimidazolium groups in relation to relatively “bulky” groups at the R₁ positions on the aryl ring. Accordingly, as noted above, the R_{1AA} and R_{1XX} groups are at least as large as a methyl group.

The moieties can be incorporated into a polymer in any manner known to those of skill in the art. Particularly, the moieties can be attached to a polymer chain at any of the R_{1AA}, R_{2AA}, R_{3AA}, R_{5AA}, R_{1XX}, R_{2XX}, R_{3XX}, and R_{5XX} positions. As used herein, when an R-group is defined as a “polymer”, that R-group location connects one of the moieties to a polymer chain. As discussed further herein, multiple R-groups can be “polymer” and the moieties can be incorporated into a polymer in a number of ways, including as part of the polymer backbone and/or as a pendant.

In one embodiment, the moiety M1 is incorporated in the monomer as part of the polymer backbone, as described in further experimental detail below. As used herein, a monomer that is part of the main chain (or backbone) of a polymer is a repeating unit that is connected on at least two ends to the polymer chain. It will be appreciated that the moiety can be the only moiety in the backbone monomer: M1ₓ. Alternatively, the moiety can be one of a plurality of moieties in the backbone of the monomer: [M1]ₓ[A]_{y}[B]_{z},

As an example of integrating the moiety M1 into the main chain of a polymer, the following exemplary structure of Formula (I-A) is illustrative:

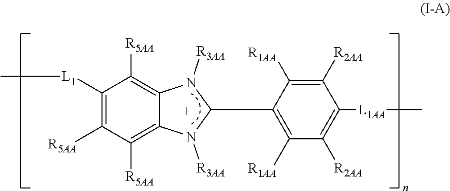

wherein L_{1AA} is an optional addition group or groups on either side of the moiety. L_{1AA} can be any group known to those of skill in the art (e.g., alkyl, aryl, etc.).

In one embodiment, the moiety M1 is incorporated as a pendant moiety attached to the backbone of the polymer. As used herein, the term “pendant” refers to a moiety that is attached at only one end to a polymer backbone. It will be appreciated that the moiety may be directly connected to the polymer backbone or there may be additional moieties (e.g., linker groups, L_{2AA}) in between the moiety and the polymer backbone. Once again, attachment can come at any of the R_{1AA}, R_{2AA}, R_{3AA}, or R_{5AA} positions.

For example, attachment can be through an R_{2AA} position on the aryl, as illustrated in Formula (I-B) below. L_{2AA} is an optional linker group connecting the main chain of the polymer, P_{1AA}, to the moiety. L_{2AA} can be any group known to those of skill in the art.

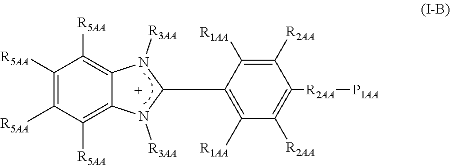

Alternatively, attachment can be through an R_{3AA} position on the benzimidazolium, as illustrated in Formula (I-C) below.

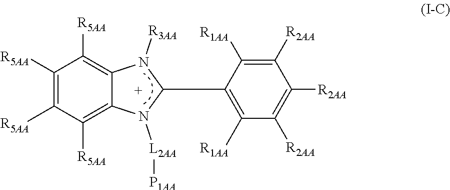

In one embodiment, the moiety M2 is incorporated as a pendant moiety attached to the backbone of the polymer. It will be appreciated that the moiety may be directly connected to the polymer backbone or there may be additional moieties (e.g., linker groups, L_{2AA}) in between the moiety and the polymer backbone. Once again, attachment can come at any of the R_{1AA}, R_{2AA}, R_{3AA}, or R_{5AA} positions.

For example, attachment can be through an R_{2XX} position on the aryl, as illustrated in Formula (II-A) below.

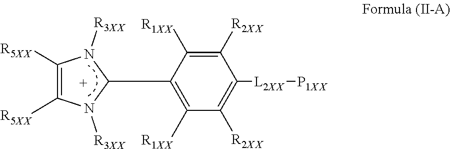

Similarly, attachment can be through an R_{3XX} position on the imidazolium, as illustrated in Formula (II-B) below.

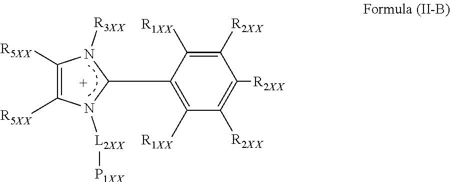

Given the multiple available locations on the moieties for attachment to a polymer main chain, the moieties can be attached to multiple polymer chains (e.g., as part of a crosslink). An exemplary embodiment illustrating crosslinking between two polymer chains, P_{1XX} and P_{2XX}, via the R_{3XX} positions, is illustrated in Formula (II-C) below. It will be appreciated that the crosslinking capabilities of the moieties are not limited to the illustrated embodiment.

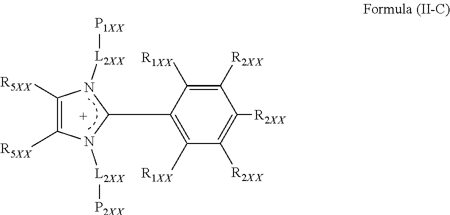

In one embodiment, the moiety M1 is grafted onto an already-formed polymer. For example, using a benzimidazole with a monosubstituted R_{3AA} position (Formula I-D), mixed with P_{1AA} and heated can produce a pendant benzimidazolium (Formula I-C) connected at the formerly vacant R_{3AA} position. Examples of P_{1AA} include alkylhalide-containing polymers such as chloromethylated polysulfone and poly(chloromethylstyrene), including perfluorinated polymers containing haloalkyl groups. In addition, perfluorinated sulfonyl halide-containing polymers, or polymers containing acyl halides can be functionalized using this method.

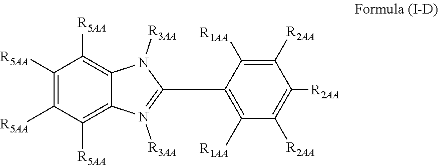

Alternatively, the moiety M2 is grafted onto an already-formed polymer using an imidazole with a monosubstituted R₃ position (Formula II-D) to produce the pendant imidazolium (Formula II-C).

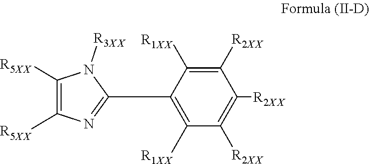

In certain embodiment, the disclosed cationic moieties form a salt with an anion. Any anion sufficient to balance the charge of the moiety-containing polymer can be used. Representative anions include iodide, triiodide, hydroxide, chloride, bromide, fluoride, cyanide, acetate, carbonate, nitrate, sulfate, phosphate, triflate, tosylate, bisulfate, bicarbonate, hydrogen phosphate, and dihydrogen phosphate.

The polymers containing the moieties can be of any size known to those of skill in the art.

In some embodiments, the polymer is a polymer of Formula III:

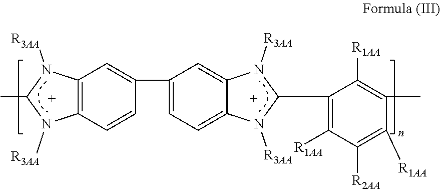

In one embodiment, in Formula (III), R_{1AA} and R_{3AA} are methyl groups and R_{2AA} is hydrogen.

In some embodiments, the polymers of the present disclosure can include one or more repeating units, wherein at least one of the repeating units includes one or more benzimidazolium-containing moieties of Formulas (IV)-(VIII):

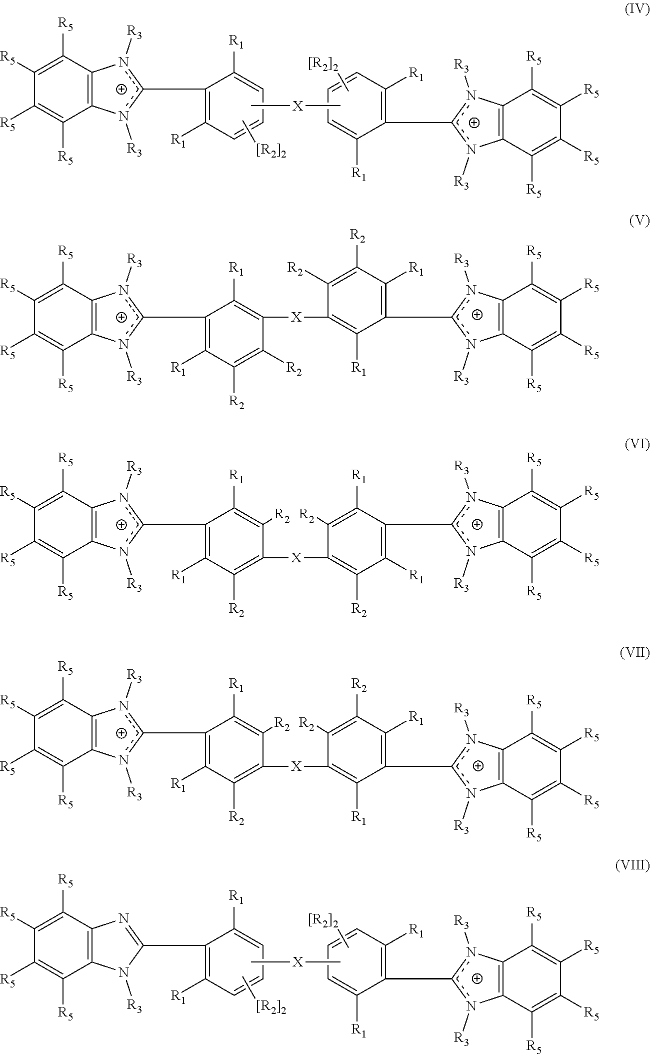

wherein:
R₁ is independently selected from the group consisting of H, methyl, trifluoromethyl, alkyl, perfluoroalkyl, heteroalkyl, alkoxy, perfluoroalkoxy, halo, aryl, heteroaryl group, and a polymer;
R₂ is independently selected from the group consisting of hydrogen, any group, and a polymer;
R₃ is independently selected from the group consisting of methyl, trifluoromethyl, alkyl, perfluoroalkyl, heteroalkyl, aryl, aralkyl, and a polymer;
R₅ is independently selected from the group consisting of hydrogen, any group, and a polymer;
wherein at least one of R₁, R₂, R₃, and R₅, is a polymer; and
X is independently selected from the group consisting of alkylene, perfluoroalkylene, heteroalkylene, arylene, aralkylene, and no group.

In some embodiments, the polymer is a salt formed with an anion selected from the group consisting of iodide, triiodide, hydroxide, chloride, bromide, fluoride, cyanide, acetate, carbonate, nitrate, sulfate, phosphate, triflate, tosylate, bicarbonate, bisulfate, hydrogen phosphate, and dihydrogen phosphate.

In some embodiments, the benzimidazolium-containing moiety is included in a main chain (i.e., the backbone) of the polymer.

In some embodiments, the benzimidazolium-containing moiety is included in a pendant group of the polymer.

In some embodiments, the benzimidazolium-containing moiety is part of a crosslink of the polymer.

In some embodiments, the polymers having at least one of the repeating units including one or more benzimidazolium-containing moieties of Formulas (IV)-(VIII) can further include a second repeating unit defined by Formula (IV-A):

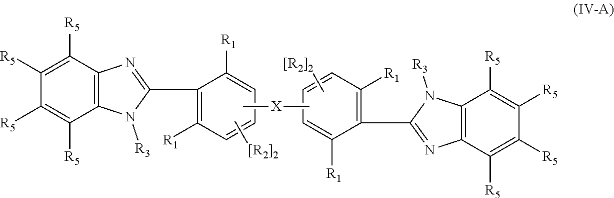

wherein:
R₁ is independently selected from the group consisting of H, methyl, trifluoromethyl, alkyl, perfluoroalkyl, heteroalkyl, alkoxy, perfluoroalkoxy, halo, aryl, heteroaryl group and a polymer;
R₂ is independently selected from the group consisting of hydrogen, any group, and a polymer;
R₃ is independently selected from the group consisting of methyl, trifluoromethyl, alkyl, perfluoroalkyl, heteroalkyl, aryl, aralkyl, and a polymer;
R₅ is independently selected from the group consisting of hydrogen, any group, and a polymer;
wherein at least one of R₁, R₂, R₃, and R₅, is a polymer; and
X is independently selected from the group consisting of alkylene, perfluoroalkylene, heteroalkylene, arylene, aralkylene, and no group.

The benzimidazolium-containing moieties can be incorporated into a polymer in any manner known to those of skill in the art. Particularly, the moieties can be attached to a polymer chain at any of the R₁, R₂, R₃, or R₅ positions. As used herein, when an R-group is defined as a “polymer”, that R-group location connects one of the benzimidazolium-containing moieties to a polymer chain. As discussed further herein, multiple R-groups can be “polymer” and the benzimidazolium-containing moieties can be incorporated into a polymer in a number of ways, including as part of the polymer backbone and/or as a pendant moiety.

In one embodiment, the benzimidazolium-containing moieties are incorporated in the polymer backbone, as described in further experimental detail below. As used herein, a monomer that is part of the main chain (or backbone) of a polymer is a repeating unit that is connected on at least two ends to the polymer chain. It will be appreciated that the moiety can be the only moiety in the backbone monomer: benzimidazolium-containing moietyₓ. Alternatively, the moiety can be one of a plurality of moieties in the backbone of the monomer: [benzimidazolium-containing moiety]ₓ[A]_{y}[B]_{z},

In one embodiment, the benzimidazolium-containing moiety is incorporated as a pendant moiety attached to the backbone of the polymer. As used herein, the term “pendant” refers to a moiety that is attached at only one end to a polymer backbone. It will be appreciated that the benzimidazolium-containing moieties may be directly connected to the polymer backbone or there may be additional moieties (e.g., linker groups) in between the moiety and the polymer backbone. Once again, attachment can come at any of the R₁, R₂, R₃, or R₅ positions.

Given the multiple available locations on the moieties for attachment to a polymer main chain, the moieties can be attached to multiple polymer chains (e.g., as part of a crosslink). An exemplary embodiment illustrating crosslinking between two polymer chains, P₁ and P₂, via the R₃ positions, is illustrated in Formula (IV-B) below. It will be appreciated that the crosslinking capabilities of the moieties are not limited to the illustrated embodiment.

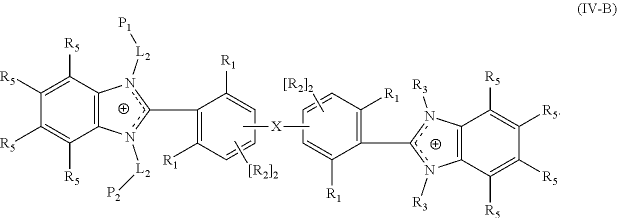

As described above, the polymer of the present disclosure includes one or more repeating units, wherein at least one of the repeating units includes one or more benzimidazolium-containing moieties of Formulas (IV)-(VIII), in any combination. In some embodiments, the polymer includes one or more repeat units, wherein at least one of the repeat units includes a benzimidazolium-containing moiety of Formula (IV). In some embodiments, the polymer includes one or more repeat units, wherein at least one of the repeat units includes a benzimidazolium-containing moiety of Formula (V). In some embodiments, the polymer includes one or more repeat units, wherein at least one of the repeat units includes a benzimidazolium-containing moiety of Formula (VI). In some embodiments, the polymer includes one or more repeat units, wherein at least one of the repeat units includes a benzimidazolium-containing moiety of Formula (VII). In some embodiments, the polymer includes one or more repeat units, wherein at least one of the repeat units includes a benzimidazolium-containing moiety of Formula (VIII).

In some embodiments, the polymer includes one or more repeat units, wherein the one or more repeat units includes benzimidazolium-containing moieties of Formulas (IV) and (V); Formulas (IV) and (VI); Formulas (IV) and (VII); Formulas (IV) and (VIII); Formulas (IV) and (IV-A); Formulas (V) and (VI); Formulas (V) and (VII); Formulas (V) and (VIII); Formulas (V) and (IV-A); Formulas (VI) and (VII); Formulas (VI) and (VIII); Formulas (VI) and (IV-A); Formulas (VII) and (VIII); Formulas (VII) and (IV-A); or Formulas (VIII) and (IV-A).

In some embodiments, the polymer includes one or more repeat units, wherein the one or more repeat units include benzimidazolium-containing moieties of 3 of Formulas (IV), (V), (VI), (VII), (VIII), and (IV-A).

In some embodiments, the polymer includes one or more repeat units, wherein the one or more repeat units include benzimidazolium-containing moieties of Formulas (IV), (VIII), and (IV-A). In some embodiments, the one or more repeat units of the polymer include benzimidazolium-containing moieties of Formulas (V), (VI), (VII), (VIII), and (IV-A). In certain embodiments, the one or more repeat units of the polymer include benzimidazolium-containing moieties of Formulas (V), (VIII), and (IV-A).

The polymers as described above can have the following embodiments and features.

In some embodiments, R₁ is independently selected from the group consisting of methyl, trifluoromethyl, alkyl, perfluoroalkyl, heteroalkyl, alkoxy, perfluoroalkoxy, halo, aryl, and heteroaryl.

In some embodiments, R₁ is independently selected from the group consisting of methyl, trifluoromethyl, alkyl, perfluoroalkyl, heteroalkyl, alkoxy, and perfluoroalkoxy.

In some embodiments, R₁ is independently selected from the group consisting of methyl, trifluoromethyl, alkyl, perfluoroalkyl, and heteroalkyl.

In some embodiments, R₁ is independently selected from the group consisting of alkyl, perfluoroalkyl, and heteroalkyl.

In some embodiments, R₁ is alkyl.

In some embodiments, R₁ is methyl.

In some embodiments, R₂ is independently selected from the group consisting of hydrogen and any group.

In some embodiments, R₂ is independently selected from the group consisting of hydrogen and alkyl.

In some embodiments, R₂ is independently selected from the group consisting of hydrogen and methyl.

In some embodiments, R₃ is independently selected from the group consisting of methyl, trifluoromethyl, alkyl, perfluoroalkyl, heteroalkyl, aryl, and aralkyl.

In some embodiments, R₃ is independently selected from the group consisting of methyl, trifluoromethyl, alkyl, perfluoroalkyl, heteroalkyl, and aryl.

In some embodiments, R₃ is independently selected from the group consisting of methyl, trifluoromethyl, alkyl, perfluoroalkyl, and heteroalkyl.

In some embodiments, R₃ is independently selected from the group consisting of alkyl, perfluoroalkyl, and heteroalkyl.

In some embodiments, R₃ is alkyl.

In some embodiments, R₃ is methyl.

In some embodiments, R₅ is independently selected from the group consisting of hydrogen, alkyl, and a polymer.

In some embodiments, R₃ is independently selected from the group consisting of hydrogen and a polymer.

In some embodiments, X is independently selected from the group consisting of alkylene, perfluoroalkylene, heteroalkylene, arylene, aralkylene, and no group.

In some embodiments, X is independently selected from the group consisting of alkylene, arylene, and aralkylene.

In some embodiments, X is independently selected from the group consisting of alkylene and arylene.

In some embodiments, X is arylene.

In some embodiments, X is phenylene (e.g., 1,4-phenylene).

The polymer can be a copolymer of Formula (IX)

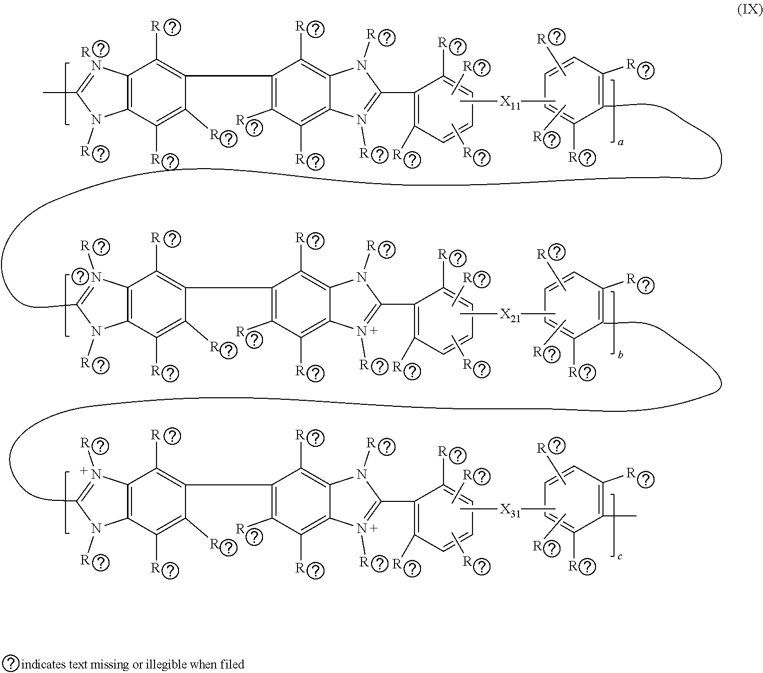

wherein:
R₁₁ₐ, R_{11b}, R_{11c}, R_{11d}, R₂₁ₐ, R_{21b}, R_{21c}, R_{21d}, R₃₁ₐ, R_{31b}, R_{31c}, and R₃₁ₐ are each independently selected from H, C₁₋₆ alkyl, C₁₋₆ haloalkyl, C₁₋₆ alkoxy, C₁₋₆ haloalkoxy, halo, aryl, and heteroaryl;
R₁₂ₐ, R_{12b}, R_{12c}, R_{12d}, R₂₂ₐ, R_{22b}, R_{22c}, R_{22d}, R₃₂ₐ, R_{32b}, R_{32c}, and R₃₂ₐ are each independently selected from H, C₁₋₆ alkyl, C₁₋₆ haloalkyl, C₁₋₆ alkoxy, C₁₋₆ haloalkoxy, halo, aryl, and heteroaryl;
R₁₃ₐ, R_{13b}, R₁₄ₐ, and R_{14b} are each independently selected from absent, C₁₋₆ alkyl, C₁₋₆ haloalkyl, C₁₋₆ alkoxy, C₁₋₆ haloalkoxy, halo, aryl, and heteroaryl, provided that two of R₁₃ₐ, R_{13b}, R₁₄ₐ, and R_{14b} are absent, and the remaining two are present and independently selected from C₁₋₆ alkyl, C₁₋₆ haloalkyl, C₁₋₆ alkoxy, C₁₋₆ haloalkoxy, halo, aryl, and heteroaryl.
R₂₃ₐ, R_{23b}, R₂₄ₐ, and R_{24b} are each independently selected from absent, C₁₋₆ alkyl, C₁₋₆ haloalkyl, C₁₋₆ alkoxy, C₁₋₆ haloalkoxy, halo, aryl, and heteroaryl, provided that one of R₂₃ₐ, R_{23b}, R24a and R_{24b} is absent, and the remaining three are present and independently selected from C₁₋₆ alkyl, C₁₋₆ haloalkyl, C₁₋₆ alkoxy, C₁₋₆ haloalkoxy, halo, aryl, and heteroaryl;
R₃₃ₐ, R_{33b}, R₃₄ₐ, and R_{34b} are each independently selected from C₁₋₆ alkyl, C₁₋₆ haloalkyl, C₁₋₆ alkoxy, C₁₋₆ haloalkoxy, halo, aryl, and heteroaryl;
R₁₅ₐ, R_{15b}, R_{15c}, R_{15d}, R₁₅ₑ, R_{15f}, R₂₅ₐ, R_{25b}, R_{25c}, R_{25d}, R₂₅ₑ, R_{25f}, R₃₅ₐ, R_{35b}, R_{35c}, R_{35d}, R₃₅ₑ, and R_{35f} are each independently selected from H, C₁₋₆ alkyl, C₁₋₆ haloalkyl, C₁₋₆ alkoxy, C₁₋₆ haloalkoxy, halo, aryl, and heteroaryl;
X₁₁, X₂₁, and X₃₁ are each independently selected from the group consisting of absent, C₁₋₆ alkylene, C₁₋₆ haloalkylene, arylene, and heteroarylene; and
a, b, and c are mole percentages, wherein
a is from 0 mole percent to 45 mole percent,
b+c is 55 mole percent to 100 mole percent,
b and c are each more than 0 percent, and
a+b+c=100%.

In some embodiments, the copolymer of Formula (IX) is a copolymer of Formula (IXa)

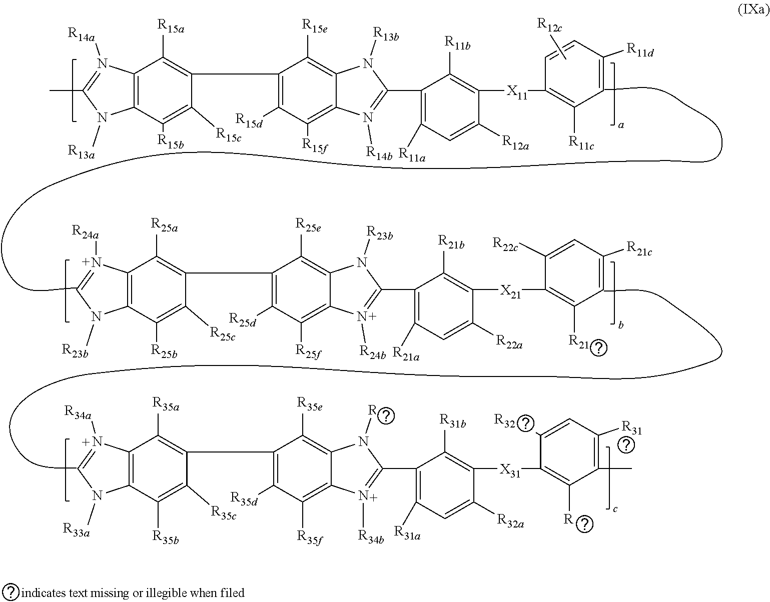

wherein:
R₁₁ₐ, R_{11b}, R_{11c}, R_{11d}, R₂₁ₐ, R_{21b}, R_{21c}, R_{21d}, R₃₁ₐ, R_{31b}, R_{31c}, and R_{31d} are each independently selected from H, C₁₋₆ alkyl, C₁₋₆ haloalkyl, C₁₋₆ alkoxy, C₁₋₆ haloalkoxy, halo, aryl, and heteroaryl;
R₁₂ₐ, R_{12c}, R₂₂ₐ, R_{22c}, R₃₂ₐ, and R_{32c} are each independently selected from H, C₁₋₆ alkyl, C₁₋₆ haloalkyl, C₁₋₆ alkoxy, C₁₋₆ haloalkoxy, halo, aryl, and heteroaryl;
R₁₃ₐ, R_{13b}, R₁₄ₐ, and R_{14b} are each independently selected from absent, C₁₋₆ alkyl, C₁₋₆ haloalkyl, C₁₋₆ alkoxy, C₁₋₆ haloalkoxy, halo, aryl, and heteroaryl, provided that two of R₁₃ₐ, R_{13b}, R₁₄ₐ, and R_{14b} are absent, and the remaining two are present and independently selected from C₁₋₆ alkyl, C₁₋₆ haloalkyl, C₁₋₆ alkoxy, C₁₋₆ haloalkoxy, halo, aryl, and heteroaryl.
R₂₃ₐ, R_{23b}, R₂₄ₐ, and R_{24b} are each independently selected from absent, C₁₋₆ alkyl, C₁₋₆ haloalkyl, C₁₋₆ alkoxy, C₁₋₆ haloalkoxy, halo, aryl, and heteroaryl, provided that one of R₂₃ₐ, R_{23b}, R₂₄ₐ and R_{24b} is absent, and the remaining three are present and independently selected from C₁₋₆ alkyl, C₁₋₆ haloalkyl, C₁₋₆ alkoxy, C₁₋₆ haloalkoxy, halo, aryl, and heteroaryl;
R₃₃ₐ, R_{33b}, R₃₄ₐ, and R_{34b} are each independently selected from C₁₋₆ alkyl, C₁₋₆ haloalkyl, C₁₋₆ alkoxy, C₁₋₆ haloalkoxy, halo, aryl, and heteroaryl;
R₁₅ₐ, R_{15b}, R_{15c}, R_{15d}, R₁₅ₑ, R_{15f}, R₂₅ₐ, R_{25b}, R_{25c}, R_{25d}, R₂₅ₑ, R_{25f}, R₃₅ₐ, R_{35b}, R_{35c}, R_{35d}, R₃₅ₑ, and R_{35f} are each independently selected from H, C₁₋₆ alkyl, C₁₋₆ haloalkyl, C₁₋₆ alkoxy, C₁₋₆ haloalkoxy, halo, aryl, and heteroaryl;
X₁₁, X₂₁, and X₃₁ are each independently selected from the group consisting of absent, C₁₋₆ alkylene, C₁₋₆ haloalkylene, arylene, and heteroarylene; and
a, b, and c are mole percentages, wherein
a is from 0 mole percent to 45 mole percent,
b+c is 55 mole percent to 100 mole percent,
b and c are each more than 0 percent, and
a+b+c=100%.

The copolymers of Formula (IX) (and Formula (IXa)) as described above can have the following embodiments and features.

In some embodiments, the copolymers of Formula (IX) (and Formula (IXa)) are random copolymers.

In some embodiments, the copolymers of Formula (IX) (and Formula (IXa)) are block copolymers. Block copolymers can be made, for example, as described in Maity S. and Jana T., Appl. Mater. Interfaces, 2014, 6 (9), pp 6851-6864. For example, two separate homopolymers can be synthesized and then reacted together in another polymerization to provide a block copolymer. Post-polymerization functionalization (described in greater detail below) can then provide block copolymers having ionic amine backbones, where N-substitution is randomly distributed along the polymer chain.

In some embodiments, R₁₁ₐ, R_{11b}, R_{11c}, R_{11d}, R₂₁ₐ, R_{21b}, R_{21c}, R_{21d}, R₃₁ₐ, R_{31b}, R_{31c}, and R_{31d} are each independently selected from C₁₋₆ alkyl, C₁₋₆ haloalkyl, C₁₋₆ alkoxy, C₁₋₆ haloalkoxy, and halo.

In some embodiments, R₁₁ₐ, R_{11b}, R_{11c}, R_{11d}, R₂₁ₐ, R_{21b}, R_{21c}, R_{21d}, R₃₁ₐ, R_{31b}, R_{31c}, and R_{31d} are each independently selected from C₁₋₆ alkyl, C₁₋₆ haloalkyl, C₁₋₆ alkoxy, and C₁₋₆ haloalkoxy.

In some embodiments, R₁₁ₐ, R_{11b}, R_{11c}, R_{11d}, R₂₁ₐ, R_{21b}, R_{21c}, R_{21d}, R₃₁ₐ, R_{31b}, R_{31c}, and R_{31d} are each independently selected from C₁₋₆ alkyl and C₁₋₆ haloalkyl,

In some embodiments, R₁₁ₐ, R_{11b}, R_{11c}, Rid. R₂₁ₐ, R_{21b}, R_{21c}, R_{21d}, R₃₁ₐ, R_{31b}, R_{31c}, and R_{31d} are each independently selected from C₁₋₆ alkyl.

In some embodiments, R₁₁ₐ, R_{11b}, R_{11c}, R_{11d}, R₂₁ₐ, R_{21b}, R_{21c}, R_{21d}, R₃₁ₐ, R_{31b}, R_{31c}, and R_{31d} are each independently selected from methyl and ethyl.

In some embodiments, R₁₁ₐ, R_{11b}, R_{11c}, R_{11d}, R₂₁ₐ, R_{21b}, R_{21c}, R_{21d}, R₃₁ₐ, R_{31b}, R_{31c}, and R_{31d} are each methyl.

In some embodiments, R₁₂ₐ, R_{12b}, R_{12c}, R_{12d}, R₂₂ₐ, R_{22b}, R_{22c}, R_{22d}, R₃₂ₐ, R_{32b}, R_{32c}, and R_{32d} are each independently selected from H, C₁₋₆ alkyl, C₁₋₆ haloalkyl, C₁₋₆ alkoxy, C₁₋₆ haloalkoxy, and halo.

In some embodiments, R₁₂ₐ, R_{12b}, R_{12c}, R_{12d}, R₂₂ₐ, R_{22b}, R_{22c}, R_{22d}, R₃₂ₐ, R_{32b}, R_{32c}, and R_{32d} are each independently selected from H, C₁₋₆ alkyl, C₁₋₆ haloalkyl, C₁₋₆ alkoxy, and C₁₋₆ haloalkoxy.

In some embodiments, R₁₂ₐ, R_{12b}, R_{12c}, R_{12d}, R₂₂ₐ, R_{22b}, R_{22c}, R_{22d}, R₃₂ₐ, R_{32b}, R_{32c}, and R_{32d} are each independently selected from H, C₁₋₆ alkyl, and C₁₋₆ haloalkyl.

In some embodiments, R₁₂ₐ, R_{12b}, R_{12c}, R_{12d}, R₂₂ₐ, R_{22b}, R_{22c}, R_{22d}, R₃₂ₐ, R_{32b}, R_{32c}, and R_{32d} are each independently selected from H and C₁₋₆ alkyl.

In some embodiments, R₁₂ₐ, R_{12b}, R_{12c}, R_{12d}, R₂₂ₐ, R_{22b}, R_{22c}, R_{22d}, R₃₂ₐ, R_{32b}, R_{32c}, and R_{32d} are each independently selected from H, methyl, and ethyl.

In some embodiments, R₁₂ₐ, R_{12b}, R_{12c}, R_{12d}, R₂₂ₐ, R_{22b}, R_{22c}, R_{22d}, R₃₂ₐ, R_{32b}, R_{32c}, and R_{32d} are each independently selected from H and methyl.

In some embodiments, R₁₂ₐ, R_{12c}, R₂₂ₐ, R_{22c}, R₃₂ₐ, and R_{32c} are each independently selected from H, C₁₋₆ alkyl, C₁₋₆ haloalkyl, C₁₋₆ alkoxy, C₁₋₆ haloalkoxy, and halo.

In some embodiments, R₁₂ₐ, R_{12c}, R₂₂ₐ, R_{22c}, R₃₂ₐ, and R_{32c} are each independently selected from H, C₁₋₆ alkyl, C₁₋₆ haloalkyl, C₁₋₆ alkoxy, and C₁₋₆ haloalkoxy.

In some embodiments, R₁₂ₐ, R_{12c}, R₂₂ₐ, R_{22c}, R₃₂ₐ, and R₃₂ₑ are each independently selected from H, C₁₋₆ alkyl, and C₁₋₆ haloalkyl.

In some embodiments, R₁₂ₐ, R_{12c}, R₂₂ₐ, R_{22c}, R₃₂ₐ, and R_{32c} are each independently selected from H and C₁₋₆ alkyl.

In some embodiments, R₁₂ₐ, R_{12c}, R₂₂ₐ, R_{22c}, R₃₂ₐ, and R_{32c} are each independently selected from H, methyl, and ethyl.

In some embodiments, R₁₂ₐ, R_{12c}, R₂₂ₐ, R_{22c}, R₃₂ₐ, and R_{32c} are each independently selected from H and methyl.

In some embodiments, R₁₃ₐ, R_{13b}, R₁₄ₐ, and R_{14b} are each independently selected from absent, C₁₋₆ alkyl, C₁₋₆ haloalkyl, C₁₋₆ alkoxy, C₁₋₆ haloalkoxy, and halo, provided that two of R₁₃ₐ, R_{13b}, R₁₄ₐ, and R_{14b} are absent, and the remaining two are present and independently selected from C₁₋₆ alkyl, C₁₋₆ haloalkyl, C₁₋₆ alkoxy, C₁₋₆ haloalkoxy, and halo.

In some embodiments, R₁₃ₐ, R_{13b}, R₁₄ₐ, and R_{14b} are each independently selected from absent, C₁₋₆ alkyl, C₁₋₆ haloalkyl, C₁₋₆ alkoxy, and C₁₋₆ haloalkoxy, provided that two of R₁₃ₐ, R_{13b}, R₁₄ₐ, and R_{14b} are absent, and the remaining two are present and independently selected from C₁₋₆ alkyl, C₁₋₆ haloalkyl, C₁₋₆ alkoxy, and C₁₋₆ haloalkoxy.

In some embodiments, R₁₃ₐ, R_{13b}, R₁₄ₐ, and R_{14b} are each independently selected from absent, C₁₋₆ alkyl, and C₁₋₆ haloalkyl, provided that two of R₁₃ₐ, R_{13b}, R₁₄ₐ, and R_{14b} are absent, and the remaining two are present and independently selected from C₁₋₆ alkyl and C₁₋₆ haloalkyl.

In some embodiments, R₁₃ₐ, R_{13b}, R₁₄ₐ, and R_{14b} are each independently selected from absent and C₁₋₆ alkyl, provided that two of R₁₃ₐ, R_{13b}, R₁₄ₐ, and R_{14b} are absent, and the remaining two are present and independently selected from C₁₋₆ alkyl.

In some embodiments, R₁₃ₐ, R_{13b}, R₁₄ₐ, and R_{14b} are each independently selected from absent, methyl, and ethyl, provided that two of R₁₃ₐ, R_{13b}, R₁₄ₐ, and R_{14b} are absent, and the remaining two are present and independently selected from methyl and ethyl.

In some embodiments, R₁₃ₐ, R_{13b}, R₁₄ₐ, and R_{14b} are each independently selected from absent and methyl, provided that two of R₁₃ₐ, R_{13b}, R₁₄ₐ, and R_{14b} are absent, and the remaining two are present and are methyl.

In some embodiments, R₂₃ₐ, R_{23b}, R₂₄ₐ, and R_{24b} are each independently selected from absent, C₁₋₆ alkyl, C₁₋₆ haloalkyl, C₁₋₆ alkoxy, C₁₋₆ haloalkoxy, and halo, provided that one of R₂₃ₐ, R_{23b}, R₂₄ₐ, and R_{24b} is absent, and the remaining three are present and independently selected from C₁₋₆ alkyl, C₁₋₆ haloalkyl, C₁₋₆ alkoxy, C₁₋₆ haloalkoxy, and halo.

In some embodiments, R₂₃ₐ, R_{23b}, R₂₄ₐ, and R_{24b} are each independently selected from absent, C₁₋₆ alkyl, C₁₋₆ haloalkyl, C₁₋₆ alkoxy, and C₁₋₆ haloalkoxy, provided that one of R₂₃ₐ, R_{23b}, R₂₄ₐ, and R_{24b} is absent, and the remaining three are present and independently selected from C₁₋₆ alkyl, C₁₋₆ haloalkyl, C₁₋₆ alkoxy, and C₁₋₆ haloalkoxy.

In some embodiments, R₂₃ₐ, R_{23b}, R₂₄ₐ, and R_{24b} are each independently selected from absent, C₁₋₆ alkyl, and C₁₋₆ haloalkyl, provided that one of R₂₃ₐ, R_{23b}, R₂₄ₐ, and R_{24b} is absent, and the remaining three are present and independently selected from C₁₋₆ alkyl and C₁₋₆ haloalkyl.

In some embodiments, R₂₃ₐ, R_{23b}, R₂₄ₐ, and R_{24b} are each independently selected from absent and C₁₋₆ alkyl, provided that one of R₂₃ₐ, R_{23b}, R₂₄ₐ, and R_{24b} is absent, and the remaining three are present and independently selected from C₁₋₆ alkyl.

In some embodiments, R₂₃ₐ, R_{23b}, R₂₄ₐ, and R_{24b} are each independently selected from absent, methyl, and ethyl, provided that one of R₂₃ₐ, R_{23b}, R₂₄ₐ, and R_{24b} is absent, and the remaining three are present and independently selected from methyl and ethyl.

In some embodiments, R₂₃ₐ, R_{23b}, R₂₄ₐ, and R_{24b} are each independently selected from absent and methyl, provided that one of R₂₃ₐ, R_{23b}, R₂₄ₐ, and R_{24b} is absent, and the remaining three are present and are methyl.

In some embodiments, R₃₃ₐ, R_{33b}, R₃₄ₐ, and R_{34b} are each independently selected from C₁₋₆ alkyl, C₁₋₆ haloalkyl, C₁₋₆ alkoxy, C₁₋₆ haloalkoxy, and halo.

In some embodiments, R₃₃ₐ, R_{33b}, R₃₄ₐ, and R_{34b} are each independently selected from C₁₋₆ alkyl, C₁₋₆ haloalkyl, C₁₋₆ alkoxy, and C₁₋₆ haloalkoxy.

In some embodiments, R₃₃ₐ, R_{33b}, R₃₄ₐ, and R_{34b} are each independently selected from C₁₋₆ alkyl and C₁₋₆ haloalkyl.

In some embodiments, R₃₃ₐ, R_{33b}, R₃₄ₐ, and R_{34b} are each independently selected from C₁₋₆ alkyl.

In some embodiments, R₃₃ₐ, R_{33b}, R₃₄ₐ, and R_{34b} are each independently selected from methyl and ethyl.

In some embodiments, R₃₃ₐ, R_{33b}, R₃₄ₐ, and R_{34b} are each independently methyl.

In some embodiments, R₁₅ₐ, R_{15b}, R_{15c}, R_{15d}, R₁₅ₑ, R_{15f}, R₂₅ₐ, R_{25b}, R_{25c}, R_{25d}, R₂₅ₑ, R_{25f}, R₃₅ₐ, R_{35b}, R_{35c}, R_{35d}, R₃₅ₑ, and R_{35f} are each independently selected from H, C₁₋₆ alkyl, C₁₋₆ haloalkyl, C₁₋₆ alkoxy, C₁₋₆ haloalkoxy, and halo.

In some embodiments, R₁₅ₐ, R_{15b}, R_{15c}, R_{15d}, R₁₅ₑ, R_{15f}, R₂₅ₐ, R_{25b}, R_{25c}, R_{25d}, R₂₅ₑ, R_{25f}, R₃₅ₐ, R_{35b}, R_{35c}, R_{35d}, R₃₅ₑ, and R_{35f} are each independently selected from H, C₁₋₆ alkyl, and C₁₋₆ haloalkyl.

In some embodiments, R₁₅ₐ, R_{15b}, R_{15c}, R_{15d}, R₁₅ₑ, R_{15f}, R₂₅ₐ, R_{25b}, R_{25c}, R_{25d}, R₂₅ₑ, R_{25f}, R₃₅ₐ, R_{35b}, R_{35c}, R_{35d}, R₃₅ₑ, and R_{35f} are each independently selected from H and C₁₋₆ alkyl.

In some embodiments, R₁₅ₐ, R_{15b}, R_{15c}, R_{15d}, R₁₅ₑ, R_{15f}, R₂₅ₐ, R_{25b}, R_{25c}, R_{25d}, R₂₅ₑ, R_{25f}, R₃₅ₐ, R_{35b}, R_{35c}, R_{35d}, R₃₅ₑ, and R_{35f} are each H.

In some embodiments, X₁₁, X₂₁, and X₃₁ are each independently selected from the group consisting of C₁₋₆ alkylene, C₁₋₆ haloalkylene, arylene, and heteroarylene.

In some embodiments, X₁₁, X₂₁, and X₃₁ are each independently selected from the group consisting of arylene and heteroarylene.

In some embodiments, X₁₁, X₂₁, and X₃₁ are each independently selected from arylene.

In some embodiments, X₁₁, X₂₁, and X₃₁ are each phenylene (e.g., 1,4-phenylene).

In some embodiments, a, b, and c are mole percentages, wherein
a is 5 mole percent or more,
b+c is 95 mole percent or less,
b and c are each more than 0 percent, and
a+b+c=100%.

In some embodiments, a, b, and c are mole percentages, wherein
a is from 5 mole percent to 45 mole percent,
b+c is 55 mole percent to 95 mole percent,
b and c are each more than 0 percent, and
a+b+c=100%.

In some embodiments, a, b, and c are mole percentages, wherein
a is from 2 mole percent to 45 mole percent,
b+c is 55 mole percent to 98 mole percent,
b and c are each more than 0 percent, and
a+b+c=100%.

In some embodiments, a, b, and c are mole percentages, wherein
a is from 0 mole percent to 45 mole percent,
b+c is 55 mole percent to 100 mole percent,
b and c are each more than 0 percent, and
a+b+c=100%.

In some embodiments, R₁₁ₐ, R_{11b}, R_{11c}, R_{11d}, R₂₁ₐ, R_{21b}, R_{21c}, R_{21d}, R₃₁ₐ, R_{31b}, R_{31c}, and R_{31d} are each independently selected from C₁₋₆ alkyl; R₁₂ₐ, R_{12c}, R₂₂ₐ, R_{22c}, R₃₂ₐ, and R_{32c} are each independently selected from H and C₁₋₆ alkyl; R₁₃ₐ, R_{13b}, R₁₄ₐ, and R_{14b} are each independently selected from absent, C₁₋₆ alkyl, and C₁₋₆ haloalkyl, provided that two of R₁₃ₐ, R_{13b}, R₁₄ₐ, and R_{14b} are absent, and the remaining two are present and independently selected from C₁₋₆ alkyl and C₁₋₆ haloalkyl; R₂₃ₐ, R_{23b}, R₂₄ₐ, and R_{24b} are each independently selected from absent, C₁₋₆ alkyl, and C₁₋₆ haloalkyl, provided that one of R₂₃ₐ, R_{23b}, R₂₄ₐ, and R_{24b} is absent, and the remaining three are present and independently selected from C₁₋₆ alkyl and C₁₋₆ haloalkyl; R₃₃ₐ, R_{33b}, R₃₄ₐ, and R_{34b} are each independently selected from C₁₋₆ alkyl and C₁₋₆ haloalkyl; R₁₅ₐ, R_{15b}, R_{15c}, R_{15d}, R₁₅ₑ, R_{15f}, R₂₅ₐ, R_{25b}, R_{25c}, R_{25d}, R₂₅ₑ, R_{25f}, R₃₅ₐ, R_{35b}, R_{35c}, R_{35d}, R₃₅ₑ, and R_{35f} are each independently selected from H and C₁₋₆ alkyl; X₁₁, X₂₁, and X₃₁ are each independently selected from arylene; a, b, and c are mole percentages, wherein a is from 2 mole percent to 45 mole percent, b+c is 55 mole percent to 98 mole percent, b and c are each more than 0 percent, and a+b+c=100%.

In some embodiments, R₁₁ₐ, R_{11b}, R_{11c}, R_{11d}, R₂₁ₐ, R_{21b}, R_{21c}, R_{21d}, R₃₁ₐ, R_{31b}, R_{31c}, and R_{31d} are each independently selected from C₁₋₆ alkyl; R₁₂ₐ, R_{12c}, R₂₂ₐ, R_{22c}, R₃₂ₐ, and R_{32c} are each independently selected from H and C₁₋₆ alkyl; R₁₃ₐ, R_{13b}, R₁₄ₐ, and R_{14b} are each independently selected from absent and C₁₋₆ alkyl, provided that two of R₁₃ₐ, R_{13b}, R₁₄ₐ, and R_{14b} are absent, and the remaining two are present and independently selected from C₁₋₆ alkyl; R₂₃ₐ, R_{23b}, R₂₄ₐ, and R_{24b} are each independently selected from absent and C₁₋₆ alkyl, provided that one of R₂₃ₐ, R_{23b}, R₂₄ₐ, and R_{24b} is absent, and the remaining three are present and independently selected from C₁₋₆ alkyl; R₃₃ₐ, R_{33b}, R₃₄ₐ, and R_{34b} are each independently selected from C₁₋₆ alkyl; R₁₅ₐ, R_{15b}, R_{15c}, R_{15d}, R₁₅ₑ, R_{15f}, R₂₅ₐ, R_{25b}, R_{25c}, R_{25d}, R₂₅ₑ, R_{25f}, R₃₅ₐ, R_{35b}, R_{35c}, R_{35d}, R_{35c}, and R_{35f} are each independently selected from H and C₁₋₆ alkyl; X₁₁, X₂₁, and X₃₁ are each independently selected from arylene; a, b, and c are mole percentages, wherein a is from 2 mole percent to 45 mole percent, b+c is 55 mole percent to 98 mole percent, b and c are each more than 0 percent, and a+b+c=100%.

In some embodiments, R₁₁ₐ, R_{11b}, R_{11c}, R_{11d}, R₂₁ₐ, R_{21b}, R_{21c}, R_{21d}, R₃₁ₐ, R_{31b}, R_{31c}, and R_{31d} are each independently selected from methyl and ethyl; R₁₂ₐ, R_{12c}, R₂₂ₐ, R_{22c}, R₃₂ₐ, and R_{32c} are each independently selected from H, methyl, and ethyl; R₁₃ₐ, R_{13b}, R₁₄ₐ, and R_{14b} are each independently selected from absent, methyl, and ethyl, provided that two of R₁₃ₐ, R_{13b}, R₁₄ₐ, and R_{14b} are absent, and the remaining two are present and independently selected from methyl and ethyl; R₂₃ₐ, R_{23b}, R₂₄ₐ, and R_{24b} are each independently selected from absent, methyl, and ethyl, provided that one of R₂₃ₐ, R_{23b}, R₂₄ₐ, and R_{24b} is absent, and the remaining three are present and independently selected from methyl and ethyl; R₃₃ₐ, R_{33b}, R₃₄ₐ, and R_{34b} are each independently selected from methyl and ethyl; R₁₅ₐ, R_{15b}, R_{15c}, R_{15d}, R₁₅ₑ, R_{15f}, R₂₅ₐ, R_{25b}, R_{25c}, R_{25d}, R₂₅ₑ, R_{25f}, R₃₅ₐ, R_{35b}, R_{35c}, R_{35d}, R_{35c}, and R_{35f} are each H; X₁₁, X₂₁, and X₃₁ are each independently selected from arylene; a, b, and c are mole percentages, wherein a is from 0 mole percent to 45 mole percent, b+c is 55 mole percent to 100 mole percent, b and c are each more than 0 percent, and a+b+c=100%.

In some embodiments, R₁₁ₐ, R_{11b}, R_{11c}, R_{11d}, R₂₁ₐ, R_{21b}, R_{21c}, R_{21d}, R₃₁ₐ, R_{31b}, R_{31c}, and R_{31d} are each independently selected from methyl and ethyl; R₁₂ₐ, R_{12c}, R₂₂ₐ, R_{22c}, R₃₂ₐ, and R_{32c} are each independently selected from H, methyl, and ethyl; R₁₃ₐ, R_{13b}, R₁₄ₐ, and R_{14b} are each independently selected from absent, methyl, and ethyl, provided that two of R₁₃ₐ, R_{13b}, R₁₄ₐ, and R_{14b} are absent, and the remaining two are present and independently selected from methyl and ethyl; R₂₃ₐ, R_{23b}, R₂₄ₐ, and R_{24b} are each independently selected from absent, methyl, and ethyl, provided that one of R₂₃ₐ, R_{23b}, R₂₄ₐ, and R_{24b} is absent, and the remaining three are present and independently selected from methyl and ethyl; R₃₃ₐ, R_{33b}, R₃₄ₐ, and R_{34b} are each independently selected from methyl and ethyl; R₁₅ₐ, R_{15b}, R_{15c}, R_{15d}, R_{15c}, R_{15f}, R₂₅ₐ, R_{25b}, R_{25c}, R_{25d}, R_{25c}, R_{25f}, R₃₅ₐ, R_{35b}, R_{35c}, R_{35d}, R_{35c}, and R_{35f} are each H; X₁₁, X₂₁, and X₃₁ are each independently selected from arylene; a, b, and c are mole percentages, wherein a is from 2 mole percent to 45 mole percent, b+c is 55 mole percent to 98 mole percent, b and c are each more than 0 percent, and a+b+c=100%.

In some embodiments, R₁₁ₐ, R_{11b}, R_{11c}, R_{11d}, R₂₁ₐ, R_{21b}, R_{21c}, R_{21d}, R₃₁ₐ, R_{31b}, R_{31c}, and R_{31d} are each methyl; R₁₂ₐ, R_{12c}, R₂₂ₐ, R_{22c}, R₃₂ₐ, and R_{32c} are each independently selected from H and methyl; R₁₃ₐ, R_{13b}, R₁₄ₐ, and R_{14b} are each independently selected from absent and methyl, provided that two of R₁₃ₐ, R_{13b}, R₁₄ₐ, and R_{14b} are absent, and the remaining two are present and are methyl; R₂₃ₐ, R_{23b}, R₂₄ₐ, and R_{24b} are each independently selected from absent and methyl, provided that one of R₂₃ₐ, R_{23b}, R₂₄ₐ, and R_{24b} is absent, and the remaining three are present and are methyl; R₃₃ₐ, R_{33b}, R₃₄ₐ, and R_{34b} are each independently methyl; R₁₅ₐ, R_{15b}, R_{15c}, R_{15d}, R₁₅ₑ, R_{15f}, R₂₅ₐ, R_{25b}, R_{25c}, R_{25d}, R_{25c}, R_{25f}, R₃₅ₐ, R_{35b}, R_{35c}, R_{35d}, R₃₅ₑ, and R_{35f} are each H; X₁₁, X₂₁, and X₃₁ are each independently selected from arylene; a, b, and c are mole percentages, wherein a is from 2 mole percent to 45 mole percent, b+c is 55 mole percent to 98 mole percent, b and c are each more than 0 percent, and a+b+c=100%.

In some embodiments, R₁₁ₐ, R_{11b}, R_{11c}, R_{11d}, R₂₁ₐ, R_{21b}, R_{21c}, R_{21d}, R₃₁ₐ, R_{31b}, R_{31c}, and R_{31d} are each methyl; R₁₂ₐ, R_{12c}, R₂₂ₐ, R_{22c}, R₃₂ₐ, and R_{32c} are each independently selected from H and methyl; R₁₃ₐ, R_{13b}, R₁₄ₐ, and R_{14b} are each independently selected from absent and methyl, provided that two of R₁₃ₐ, R_{13b}, R₁₄ₐ, and R_{14b} are absent, and the remaining two are present and are methyl; R₂₃ₐ, R_{23b}, R₂₄ₐ, and R_{24b} are each independently selected from absent and methyl, provided that one of R₂₃ₐ, R_{23b}, R₂₄ₐ, and R_{24b} is absent, and the remaining three are present and are methyl; R₃₃ₐ, R_{33b}, R₃₄ₐ, and R_{34b} are each independently methyl; R₁₅ₐ, R_{15b}, R_{15c}, R_{15d}, R_{15c}, R_{15f}, R₂₅ₐ, R_{25b}, R_{25c}, R_{25d}, R₂₅ₑ, R_{25f}, R₃₅ₐ, R_{35b}, R_{35c}, R_{35d}, R_{35c}, and R_{35f} are each H; X₁₁, X₂₁, and X₃₁ are each independently selected from arylene; a, b, and c are mole percentages, wherein a is from 0 mole percent to 45 mole percent, b+c is 55 mole percent to 100 mole percent, b and c are each more than 0 percent, and a+b+c=100%.

In some embodiments, R₁₁ₐ, R_{11b}, R_{11c}, R_{11d}, R₂₁ₐ, R_{21b}, R_{21c}, R_{21d}, R₃₁ₐ, R_{31b}, R_{31c}, and R_{31d} are each methyl; R₁₂ₐ, R_{12c}, R₂₂ₐ, R_{22c}, R₃₂ₐ, and R_{32c} are each independently selected from H and methyl; R₁₃ₐ, R_{13b}, R₁₄ₐ, and R_{14b} are each independently selected from absent and methyl, provided that two of R₁₃ₐ, R_{13b}, R₁₄ₐ, and R_{14b} are absent, and the remaining two are present and are methyl; R₂₃ₐ, R_{23b}, R₂₄ₐ, and R_{24b} are each independently selected from absent and methyl, provided that one of R₂₃ₐ, R_{23b}, R₂₄ₐ, and R_{24b} is absent, and the remaining three are present and are methyl; R₃₃ₐ, R_{33b}, R₃₄ₐ, and R_{34b} are each independently methyl; R₁₅ₐ, R_{15b}, R_{15c}, R_{15d}, R_{15c}, R_{15f}, R₂₅ₐ, R_{25b}, R_{25c}, R_{25d}, R_{25c}, R_{25f}, R₃₅ₐ, R_{35b}, R_{35c}, R_{35d}, R₃₅ₑ, and R₃₅ᵣ are each H; X₁₁, X₂₁, and X₃₁ are each phenylene (e.g., 1,4-phenylene); a, b, and c are mole percentages, wherein a is from 2 mole percent to 45 mole percent, b+c is 55 mole percent to 98 mole percent, b and c are each more than 0 percent, and a+b+c=100%.

In some embodiments, R₁₁ₐ, R_{11b}, R_{11c}, R_{11d}, R₂₁ₐ, R_{21b}, R_{21c}, R_{21d}, R₃₁ₐ, R_{31b}, R_{31c}, and R_{31d} are each methyl; R₁₂ₐ, R_{12c}, R₂₂ₐ, R_{22c}, R₃₂ₐ, and R_{32c} are each independently selected from H and methyl; R₁₃ₐ, R_{13b}, R₁₄ₐ, and R_{14b} are each independently selected from absent and methyl, provided that two of R₁₃ₐ, R_{13b}, R₁₄ₐ, and R_{14b} are absent, and the remaining two are present and are methyl; R₂₃ₐ, R_{23b}, R₂₄ₐ, and R_{24b} are each independently selected from absent and methyl, provided that one of R₂₃ₐ, R_{23b}, R₂₄ₐ, and R_{24b} is absent, and the remaining three are present and are methyl; R₃₃ₐ, R_{33b}, R₃₄ₐ, and R_{34b} are each independently methyl; R₁₅ₐ, R_{15b}, R_{15c}, R_{15d}, R_{15c}, R_{15f}, R₂₅ₐ, R_{25b}, R_{25c}, R_{25d}, R_{25c}, R_{25f}, R₃₅ₐ, R_{35b}, R_{35c}, R_{35d}, R_{35c}, and R_{35f} are each H; X₁₁, X₂₁, and X₃₁ are each phenylene (e.g., 1,4-phenylene); a, b, and c are mole percentages, wherein a is from 0 mole percent to 45 mole percent, b+c is 55 mole percent to 100 mole percent, b and c are each more than 0 percent, and a+b+c=100%.

In some embodiments, the degree of N-substitution (e.g., N-alkylation) in the polymers of the present disclosure is from greater than 50 mole percent (e.g., from 60 mole percent, from 70 mole percent, from 80 mole percent, or from 90 mole percent) to about 95 mole percent (to about 92 mole percent, to about 90 mole percent, to about 80 mole percent, to about 70 mole percent, or to about 60 mole percent).

In certain embodiment, the described cationic benzimidazolium-containing moieties or the polymer of Formula (IX) or (IXa) form a salt with an anion. Any anion sufficient to balance the charge of the moiety-containing polymer can be used. Representative anions include iodide, hydroxide, chloride, bromide, fluoride, cyanide, acetate, carbonate, nitrate, sulfate, triiodide, phosphate, triflate, tosylate, bisulfate, bicarbonate, hydrogen phosphate, and dihydrogen phosphate.

The polymers containing the moieties and the polymers of Formula (IX) or (IXa) can be of any size known to those of skill in the art.

The polymers of the present disclosure are described, for example, in PCT publication No. WO2015/157848, incorporated herein by reference in its entirety.

In some embodiments, the present disclosure features a polymer including (or consisting essentially of, or consisting of) a repeating unit of Formula (X):

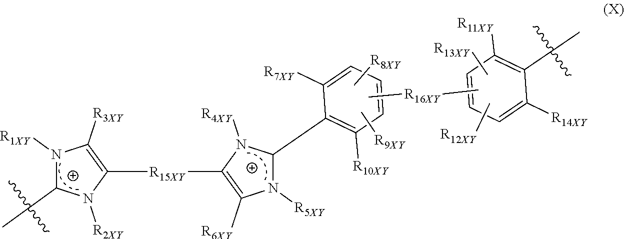

wherein:
R_{1XY}, R_{2XY}, R_{4XY}, and R_{5XY} are each independently selected from absent, alkyl, perfluoroalkyl, heteroalkyl, aryl, and aralkyl;
provided that
   at least one of R_{1XY} and R_{2XY} is selected from alkyl, perfluoroalkyl, heteroalkyl, aryl, and aralkyl,
   when one of R_{1XY} and R_{2XY} is absent, the imidazolyl group to which the absent R_{1XY} or R_{2XY} is connected (i.e., the imidazolyl group having one of R_{1XY} or R_{2XY}, but not the other) is neutral; and
   at least one of R_{4XY} and R_{5XY} is selected from alkyl, perfluoroalkyl, heteroalkyl, aryl, and aralkyl; and
   when one of R_{4XY} and R_{5XY} is absent, the imidazolyl group to which the absent R_{4XY} or R_{5XY} is connected (i.e., the imidazolyl group having one of R_{4XY} or R_{5XY}, but not the other) is neutral;
R_{3XY} and R_{6XY} are each independently selected from alkyl, perfluoroalkyl, heteroalkyl, aryl, aralkyl, and heteroaryl;
R_{15XY} is selected from alkylene, perfluoroalkylene, heteroalkylene, arylene, aralkylene, and heteroarylene, each optionally substituted with 1, 2, 3, or 4 substituents independently selected from alkyl, perfluoroalkyl, heteroalkyl, and halo;
R_{16XY} is selected from a bond, arylene, and heteroarylene, wherein said arylene and heteroarylene is each optionally substituted with 1, 2, 3, or 4 substituents independently selected from alkyl, perfluoroalkyl, heteroalkyl, and halo;
R_{7XY}, R_{10XY}, R_{11XY}, and R_{14XY} are each independently selected from H, alkyl, perfluoroalkyl, and heteroalkyl; and
R_{8XY}, R_{9XY}, R_{12XY}, and R_{13XY} are each independently selected from hydrogen (H), alkyl, perfluoroalkyl, and heteroalkyl.

In some embodiments, the polymer including (or consisting essentially of, or consisting of) a repeating unit of Formula (x) includes a repeating unit of Formula (X-A):

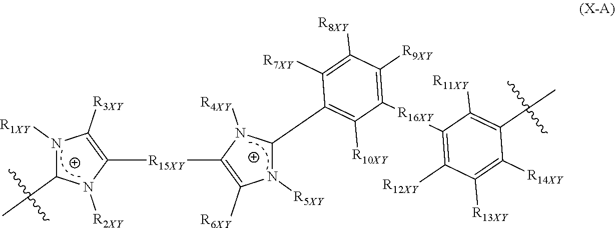

wherein:
R_{1XY}, R_{2XY}, R_{4XY}, and R_{5XY} are each independently selected from absent, alkyl, perfluoroalkyl, heteroalkyl, aryl, and aralkyl;
provided that
   at least one of R_{1XY} and R_{2XY} is selected from alkyl, perfluoroalkyl, heteroalkyl, aryl, and aralkyl,
   when one of R_{1XY} and R_{2XY} is absent, the imidazolyl group to which the absent R_{1XY} or R_{2XY} is connected (i.e., the imidazolyl group having one of R_{1XY} or R_{2XY}, but not the other) is neutral;
   at least one of R_{1XY} and R_{5XY} is selected from alkyl, perfluoroalkyl, heteroalkyl, aryl, and aralkyl; and
   when one of R_{4XY} and R_{5XY} is absent, the imidazolyl group to which the absent R_{4XY} or R_{5XY} is connected (i.e., the imidazolyl group having one of R_{4XY} or R_{5XY}, but not the other) is neutral;
R_{3XY} and R_{6XY} are each independently selected from alkyl, perfluoroalkyl, heteroalkyl, aryl, aralkyl, and heteroaryl;
R_{15XY} is selected from alkylene, perfluoroalkylene, heteroalkylene, arylene, aralkylene, and heteroarylene, each optionally substituted with 1, 2, 3, or 4 substituents independently selected from alkyl, perfluoroalkyl, heteroalkyl, and halo;
R_{16XY} is selected from a bond, arylene, and heteroarylene, wherein said arylene and heteroarylene is each optionally substituted with 1, 2, 3, or 4 substituents independently selected from alkyl, perfluoroalkyl, heteroalkyl, and halo;
R_{7XY}, R_{10XY}, R_{11XY}, and R_{14XY} are each independently selected from H, alkyl, perfluoroalkyl, and heteroalkyl; and
R_{8XY}, R_{9XY}, R_{12XY}, and R_{13XY} are each independently selected from hydrogen (H), alkyl, perfluoroalkyl, and heteroalkyl.

In some embodiments, the polymer including (or consisting essentially of, or consisting of) a repeating unit of Formula (X) includes, or the polymer including repeating unit(s) of Formula (X-A) further includes, a repeating unit of Formula (X-B):

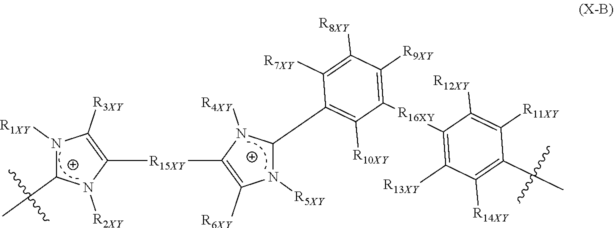

wherein:
R_{1XY}, R_{2XY}, R_{4XY}, and R_{5XY} are each independently selected from absent, alkyl, perfluoroalkyl, heteroalkyl, aryl, and aralkyl;
provided that
   at least one of R_{1XY} and R_{2XY} is selected from alkyl, perfluoroalkyl, heteroalkyl, aryl, and aralkyl,
   when one of R_{1XY} and R_{2XY} is absent, the imidazolyl group to which the absent R_{1XY} or R_{2XY} is connected (i.e., the imidazolyl group having one of R_{1XY} or R_{2XY}, but not the other) is neutral; and
   at least one of R_{4XY} and R_{5XY} is selected from alkyl, perfluoroalkyl, heteroalkyl, aryl, and aralkyl; and
   when one of R_{4XY} and R_{5XY} is absent, the imidazolyl group to which the absent R_{4XY} or R_{5XY} is connected (i.e., the imidazolyl group having one of R_{4XY} or R_{5XY}, but not the other) is neutral;
R_{3XY} and R_{6XY} are each independently selected from alkyl, perfluoroalkyl, heteroalkyl, aryl, aralkyl, and heteroaryl;
R_{15XY} is selected from alkylene, perfluoroalkylene, heteroalkylene, arylene, aralkylene, and heteroarylene, each optionally substituted with 1, 2, 3, or 4 substituents independently selected from alkyl, perfluoroalkyl, heteroalkyl, and halo;
R_{16XY} is selected from a bond, arylene, and heteroarylene, wherein said arylene and heteroarylene is each optionally substituted with 1, 2, 3, or 4 substituents independently selected from alkyl, perfluoroalkyl, heteroalkyl, and halo;
R_{7XY}, R_{10XY}, R_{11XY}, and R_{14XY} are each independently selected from H, alkyl, perfluoroalkyl, and heteroalkyl; and
R_{8XY}, R_{9XY}, R_{12XY}, and R_{13XY} are each independently selected from hydrogen (H), alkyl, perfluoroalkyl, and heteroalkyl.

In some embodiments, the polymer including (or consisting essentially of, or consisting of) a repeating unit of Formula (X) includes, or the polymer including repeating unit(s) of Formula (X-A) and/or Formula (X-B) further includes a repeating unit of Formula (X-C):

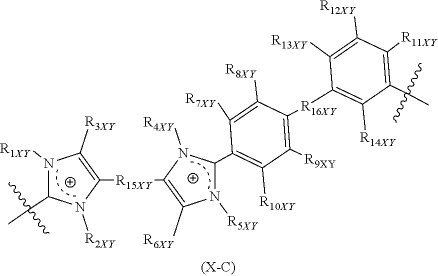

wherein:
R_{1XY}, R_{2XY}, R_{4XY}, and R_{5XY} are each independently selected from absent, alkyl, perfluoroalkyl, heteroalkyl, aryl, and aralkyl;
provided that
   at least one of R_{1XY} and R_{2XY} is selected from alkyl, perfluoroalkyl, heteroalkyl, aryl, and aralkyl,
   when one of R_{1XY} and R_{2XY} is absent, the imidazolyl group to which the absent R_{1XY} and R_{2XY} is connected (i.e., the imidazolyl group having one of R_{1XY} or R_{2XY}, but not the other) is neutral; and
   at least one of R_{4XY} and R_{5XY} is selected from alkyl, perfluoroalkyl, heteroalkyl, aryl, and aralkyl; and
   when one of R_{4XY} and R_{5XY} is absent, the imidazolyl group to which the absent R_{4XY} or R_{5XY} is connected (i.e., the imidazolyl group having one of R_{4XY} or R_{5XY}, but not the other) is neutral;
R_{3XY} and R_{6XY} are each independently selected from alkyl, perfluoroalkyl, heteroalkyl, aryl, aralkyl, and heteroaryl;
R_{15XY} is selected from alkylene, perfluoroalkylene, heteroalkylene, arylene, aralkylene, and heteroarylene, each optionally substituted with 1, 2, 3, or 4 substituents independently selected from alkyl, perfluoroalkyl, heteroalkyl, and halo;
R_{16XY} is selected from a bond, arylene, and heteroarylene, wherein said arylene and heteroarylene is each optionally substituted with 1, 2, 3, or 4 substituents independently selected from alkyl, perfluoroalkyl, heteroalkyl, and halo;
R_{7XY}, R_{10XY}, R_{11XY}, and R_{14XY} are each independently selected from H, alkyl, perfluoroalkyl, and heteroalkyl; and
R_{8XY}, R_{9XY}, R_{12XY}, and R_{13XY} are each independently selected from hydrogen (H), alkyl, perfluoroalkyl, and heteroalkyl.

In some embodiments, the polymer including (or consisting essentially of, or consisting of) a repeating unit of Formula (X) includes, or the polymer including repeating unit(s) of Formula (X-A), Formula (X-B), and/or Formula (X-C) further includes a repeating unit of Formula (X-D):

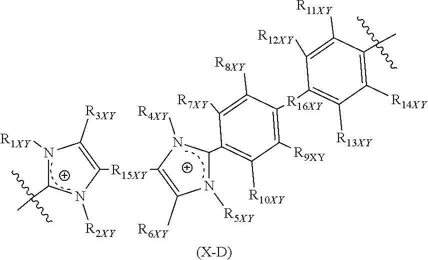

wherein:
R_{1XY}, R_{2XY}, R_{4XY}, and R_{5XY} are each independently selected from absent, alkyl, perfluoroalkyl, heteroalkyl, aryl, and aralkyl;
provided that
   at least one of R_{1XY} and R_{2XY} is selected from alkyl, perfluoroalkyl, heteroalkyl, aryl, and aralkyl,
   when one of R_{1XY} and R_{2XY} is absent, the imidazolyl group to which the absent R_{1XY} or R_{2XY} is connected (i.e., the imidazolyl group having one of R_{1XY} or R_{2XY}, but not the other) is neutral; and
   at least one of R_{4XY} and R_{5XY} is selected from alkyl, perfluoroalkyl, heteroalkyl, aryl, and aralkyl; and
   when one of R_{4XY} and R_{5XY} is absent, the imidazolyl group to which the absent R_{4XY} or R_{5XY} is connected (i.e., the imidazolyl group having one of R_{4XY} or R_{5XY}, but not the other) is neutral;
R_{3XY} and R_{6XY} are each independently selected from alkyl, perfluoroalkyl, heteroalkyl, aryl, aralkyl, and heteroaryl;
R_{15XY} is selected from alkylene, perfluoroalkylene, heteroalkylene, arylene, aralkylene, and heteroarylene, each optionally substituted with 1, 2, 3, or 4 substituents independently selected from alkyl, perfluoroalkyl, heteroalkyl, and halo;
R_{16XY} is selected from a bond, arylene, and heteroarylene, wherein said arylene and heteroarylene is each optionally substituted with 1, 2, 3, or 4 substituents independently selected from alkyl, perfluoroalkyl, heteroalkyl, and halo;
R_{7XY}, R_{10XY}, R_{11XY}, and R_{14XY} are each independently selected from H, alkyl, perfluoroalkyl, and heteroalkyl; and
R_{8XY}, R_{10XY}, R_{12XY}, and R_{13XY} are each independently selected from hydrogen (H), alkyl, perfluoroalkyl, and heteroalkyl.

The polymer of Formula (X) can have a mixture of repeating units of Formulas (X-A), (X-B), (X-C), and/or (X-D). For example, the polymer can include repeating units of Formulas (X-A), (X-B), (X-C), and (X-D); Formulas (X-A), (X-B), and (X-C); Formulas (X-A), (X-B), and (X-D); Formulas (X-A), (X-C), and (X-D); Formulas (X-B), (X-C), (X-D); Formulas (X-A) and (X-B); Formulas (X-A) and (X-C); Formulas (X-A) and (X-D); Formulas (X-B) and (X-C); Formulas (X-B) and (X-D); Formulas (X-C) and (X-D); Formula (X-A); Formula (X-B); Formula (X-C); or Formula (X-D).

In any of the above-mentioned embodiments of polymers including (or consisting essentially of, or consisting of) a repeating unit of Formula (I); or including repeating unit(s) of Formula (X-A), (X-B), (X-C), and/or (X-D), R_{1XY}, R_{2XY}, R_{4XY}, and R_{5XY} can each be independently selected from absent, alkyl, perfluoroalkyl, heteroalkyl, and aryl; provided that: at least one of R_{1XY} and R_{2XY} is selected from alkyl, perfluoroalkyl, heteroalkyl, and aryl; and at least one of R_{1XY} and R_{5XY} is selected from alkyl, perfluoroalkyl, heteroalkyl, and aryl. For example, R_{1XY}, R_{2XY}, R_{4XY}, and R_{6XY} can each independently be selected from absent, alkyl, perfluoroalkyl, and heteroalkyl; provided that: at least one of R_{1XY} and R_{2XY} is selected from alkyl, perfluoroalkyl, and heteroalkyl; and at least one of R_{4XY} and Roxy is selected from alkyl, perfluoroalkyl, and heteroalkyl. In some embodiments, R_{1XY}, R_{2XY}, R_{4XY}, and R_{5XY} are each independently selected from absent, methyl, and trifluoromethyl; provided that: at least one of R_{1XY} and R_{2XY} is selected from methyl and trifluoromethyl; and at least one of R_{1XY} and R_{5XY} is selected from methyl and trifluoromethyl.

In any of the above-mentioned embodiments of polymers including (or consisting essentially of, or consisting of) a repeating unit of Formula (X); or including repeating unit(s) of Formula (X-A), (X-B), (X-C), and/or (X-D), R_{3XY} and R_{6XY} can each independently aryl. For example, R_{3XY} and R_{6XY} can each independently phenyl. In some embodiments, R_{3XY} and R_{6XY} are each independently ethyl or methyl. In some embodiments, R_{3XY} and R_{6XY} are each independently methyl.

In any of the above-mentioned embodiments of polymers including (or consisting essentially of, or consisting of) a repeating unit of Formula (X); or including repeating unit(s) of Formula (X-A), (X-B), (X-C), and/or (X-D), R_{15XY} and R_{16XY} can each independently be selected from arylene and heteroarylene, each optionally substituted with 1, 2, 3, or 4 substituents independently selected from alkyl and halo. For example, R_{15XY} and R_{16XY} can each independently be arylene, optionally substituted with 1, 2, 3, or 4 substituents independently selected from alkyl and halo. For example, R_{15XY} and R_{16XY} can each be phenylene, optionally substituted with 1, 2, 3, or 4 substituents independently selected from alkyl and halo. In some embodiments, R_{15XY} and R_{16XY} are each phenylene.

In any of the above-mentioned embodiments of polymers including (or consisting essentially of, or consisting of) a repeating unit of Formula (X); or including repeating unit(s) of Formula (X-A), (X-B), (X-C), and/or (X-D), R_{7XY}, R_{10XY}, R_{11XXY}, and R_{14XY} can each independently be alkyl. For example, R_{7XY}, R_{10XY}, R_{11XY}, and R_{14XY} can each independently be methyl or ethyl. For example, R_{7XY}, R_{10XY}, R_{11XY}, and R_{14XY} can each independently be methyl.

In any of the above-mentioned embodiments of polymers including (or consisting essentially of, or consisting of) a repeating unit of Formula (X); or including repeating unit(s) of Formula (X-A), (X-B), (X-C), and/or (X-D), the polymer can include a counterion selected from the group consisting of iodide, triiodide, hydroxide, chloride, bromide, fluoride, cyanide, acetate, carbonate, nitrate, sulfate, phosphate, triflate, tosylate, bisulfate, bicarbonate, hydrogen phosphate, and dihydrogen phosphate. In any of the above-mentioned embodiments of polymers including (or consisting essentially of, or consisting of) a repeating unit of Formula (X); or including repeating unit(s) of Formula (X-A), (X-B), (X-C), and/or (X-D), the polymer can include one or more anions X⁻ selected from iodide, bromide, chloride, fluoride, triiodide, hydroxide, carbonate, bicarbonate, cyanide, acetate, nitrate, sulfate, phosphate, triflate, tosylate, tetrakis(3,5-bis(trifluoromethyl)phenyl) borate, bis(trifluoromethane) sulfonamide, and any combination thereof, the one or more anions X-counterbalance one or more positive charges in the polymer. In some embodiments, the polymer includes one or more anions X⁻ selected from iodide, bromide, chloride, fluoride, triiodide, hydroxide, carbonate, bicarbonate, sulfate, phosphate, triflate, tosylate, tetrakis(3,5-bis(trifluoromethyl) phenyl) borate, bis(trifluoromethane) sulfonamide, and any combination thereof, where the one or more anions X-counterbalance one or more positive charges in the polymer. In some embodiments, the polymer includes one or more anions X⁻ selected from iodide, bromide, chloride, fluoride, hydroxide, carbonate, bicarbonate, and any combination thereof, where the one or more anions X-counterbalance one or more positive charges in the polymer. In some embodiments, the polymer includes one or more anions X⁻ selected from iodide, bromide, chloride, hydroxide, and any combination thereof, where the one or more anions X-counterbalance one or more positive charges in the polymer. In some embodiments, the polymer includes one or more anions X⁻ selected from iodide, hydroxide, and any combination thereof, where the one or more anions X-counterbalance one or more positive charges in the polymer. In some embodiments, the polymer includes one or more hydroxide, where the one or more hydroxide counterbalance one or more positive charges in the polymer.

The present disclosure also provides a polymer including (or consisting essentially of, or consisting of) a repeating unit of Formula (XI):

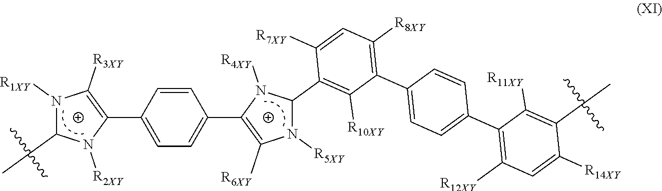

wherein:
R_{1XY}, R_{2XY}, R_{4XY}, and R_{5XY} are each independently selected from absent, alkyl, perfluoroalkyl, heteroalkyl, aryl, and aralkyl;
provided that
   at least one of R_{1XY} and R_{2XY} is selected from alkyl, perfluoroalkyl, heteroalkyl, aryl, and aralkyl,
   when one of R_{1XY} and R_{2XY} is absent, the imidazolyl group to which the absent R_{1XY} or R_{2XY} is connected (i.e., the imidazolyl group having one of R_{1XY} or R_{2XY}, but not the other) is neutral; and
   at least one of R_{4XY} and R_{5XY} is selected from alkyl, perfluoroalkyl, heteroalkyl, aryl, and aralkyl; and
   when one of R_{4XY} and R_{5XY} is absent, the imidazolyl group to which the absent R_{4XY} or R_{5XY} is connected (i.e., the imidazolyl group having one of R_{4XY} or R_{5XY}, but not the other) is neutral;
R_{3XY} and R_{6XY} are each independently selected from alkyl, perfluoroalkyl, heteroalkyl, aryl, aralkyl, and heteroaryl;
R_{7XY}, R_{10XY}, R_{11XY}, and R_{14XY} are each independently selected from H, alkyl, perfluoroalkyl, and heteroalkyl; and
R_{8XY} and R_{12XY} are each independently selected from hydrogen (H), alkyl, perfluoroalkyl, and heteroalkyl.

In some embodiments, for the above-described polymer including (or consisting essentially of, or consisting of) a repeating unit of Formula (XI), R_{1XY}, R_{2XY}, R_{4XY}, and Roxy are each independently selected from absent, alkyl, perfluoroalkyl, and heteroalkyl; provided that: at least one of R_{1XY} and R_{2XY} is selected from alkyl, perfluoroalkyl, and heteroalkyl, and at least one of R_{4XY} and R_{5XY} is selected from alkyl, perfluoroalkyl, and heteroalkyl. For example, R_{1XY}, R_{2XY}, R_{4XY}, and R_{5XY} can each independently selected from absent, methyl, and trifluoromethyl; provided that: at least one of R_{1XY} and R_{2XY} is selected from methyl and trifluoromethyl, and at least one of R_{1XY} and R_{5XY} is selected from methyl and trifluoromethyl.

In some embodiments, for any of the above-mentioned embodiments of polymers including (or consisting essentially of, or consisting of) a repeating unit of Formula (XI), R_{3XY} and R_{6XY} are each independently aryl. For example, R_{3XY} and R_{6XY} can each be independently phenyl. In some embodiments, R_{3XY} and R_{6XY} are each independently methyl or ethyl. In some embodiments, R_{3XY} and R_{6XY} are each independently methyl.

In some embodiments, for any of the above-mentioned embodiments of polymers including (or consisting essentially of, or consisting of) a repeating unit of Formula (XI), R_{7XY}, R_{8XY}, R_{10XY}, R_{11XY}, R_{12XY}, and R_{14XY} are each independently alkyl. For example, R_{7XY}, R_{8XY}, R_{10XY}, R_{11XY}, R_{12XY}, and R_{14XY} are each independently methyl or ethyl. For example, R_{7XY}, ROXY, R_{10XY}, R_{11XY}, R_{12XY}, and R_{14XY} are each independently methyl.

In some embodiments, for any of the above-mentioned embodiments of polymers including (or consisting essentially of, or consisting of) a repeating unit of Formula (XI), the polymer includes one or more counterions selected from the group consisting of iodide, triiodide, hydroxide, chloride, bromide, fluoride, cyanide, acetate, carbonate, nitrate, sulfate, phosphate, triflate, tosylate, bisulfate, bicarbonate, hydrogen phosphate, and dihydrogen phosphate. In some embodiments, for any of the above-mentioned embodiments of polymers including (or consisting essentially of, or consisting of) a repeating unit of Formula (XI), the polymer includes one or more anions X⁻ selected from iodide, bromide, chloride, fluoride, triiodide, hydroxide, carbonate, bicarbonate, cyanide, acetate, nitrate, sulfate, phosphate, triflate, tosylate, tetrakis(3,5-bis(trifluoromethyl)phenyl) borate, bis(trifluoromethane) sulfonamide, and any combination thereof, where the one or more anions X⁻ counterbalance one or more positive charges in the polymer. For example, the one or more anions X⁻ can be selected from iodide, bromide, chloride, fluoride, triiodide, hydroxide, carbonate, bicarbonate, sulfate, phosphate, triflate, tosylate, tetrakis(3,5-bis(trifluoromethyl) phenyl) borate, bis(trifluoromethane) sulfonamide, and any combination thereof, where the one or more anions X⁻ counterbalance one or more positive charges in the polymer. In some embodiments, one or more anions X⁻ are selected from iodide, bromide, chloride, fluoride, hydroxide, carbonate, bicarbonate, and any combination thereof, where the one or more anions X⁻ counterbalance one or more positive charges in the polymer. In some embodiments, the polymer includes one or more anions X⁻ selected from iodide, bromide, chloride, hydroxide, and any combination thereof, where the one or more anions X⁻ counterbalance one or more positive charges in the polymer. In some embodiments, the polymer includes one or more anions X⁻ selected from iodide, hydroxide, and any combination thereof, where the one or more anions X⁻ counterbalance one or more positive charges in the polymer. In some embodiments, the polymer includes one or more hydroxide, where the one or more hydroxide counterbalance one or more positive charges in the polymer.

The present disclosure further provides a polymer including a repeating unit of Formula (XII-A):

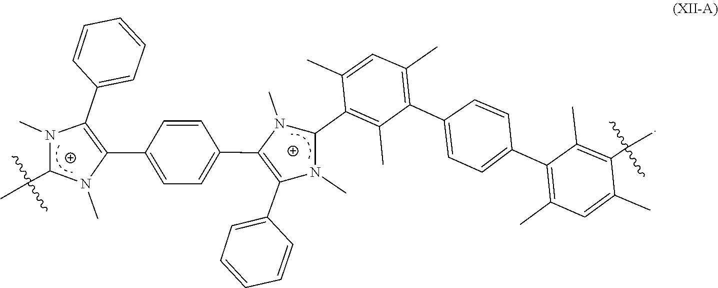

In some embodiments, the polymer including a repeating unit of Formula (XII-A) further includes a repeating unit of Formula (XII-B):

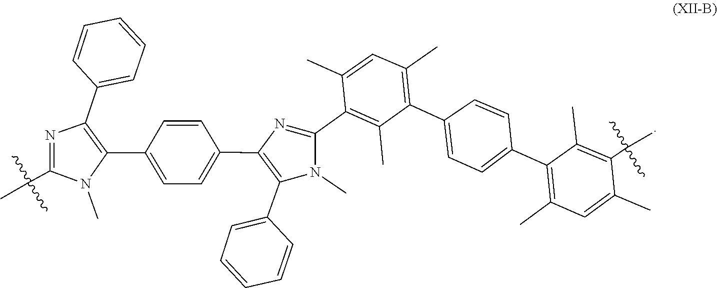

In some embodiments, the polymer including a repeating unit of Formula (XII-A), or including repeating units of Formulas (XII-A) and (XII-B), further includes a repeating unit of Formula (XII-C):

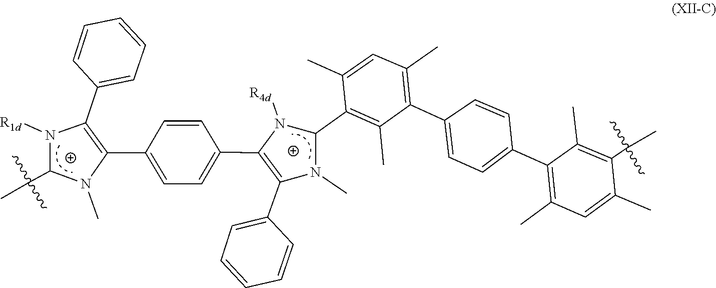

wherein one of R_{1d} and R_{4d} is absent, and the remaining R_{1d} or R_{4d} is methyl; and the imidazolyl group to which the absent R_{1d} or R_{4d} is connected (i.e., the imidazolyl group where one of its R_{1d} or R_{4d} is absent) is neutral.

In some embodiments, for any of the above-described polymers including (or consisting essentially of, or consisting of) a repeating unit of Formula (XII-A), (XII-B), and/or (XII-C), the polymer includes one or more counterions selected from the group consisting of iodide, triiodide, hydroxide, chloride, bromide, fluoride, cyanide, acetate, carbonate, nitrate, sulfate, phosphate, triflate, tosylate, bisulfate, bicarbonate, hydrogen phosphate, and dihydrogen phosphate. In some embodiments, for any of the above-described polymers including (or consisting essentially of, or consisting of) a repeating unit of Formula (XII-A), (XII-B), and/or (XII-C), the polymer includes one or more anions X⁻ selected from iodide, bromide, chloride, fluoride, triiodide, hydroxide, carbonate, bicarbonate, cyanide, acetate, nitrate, sulfate, phosphate, triflate, tosylate, tetrakis(3,5-bis(trifluoromethyl) phenyl) borate, bis(trifluoromethane) sulfonamide, and any combination thereof, where the one or more anions X⁻ counterbalances one or more positive charges in the polymer. In some embodiments, one or more anions X⁻ are selected from iodide, bromide, chloride, fluoride, triiodide, hydroxide, carbonate, bicarbonate, sulfate, phosphate, triflate, tosylate, tetrakis(3,5-bis(trifluoromethyl)phenyl) borate, bis(trifluoromethane) sulfonamide, and any combination thereof, where the one or more anions X⁻ counterbalance one or more positive charges in the polymer. For example, one or more anions X-can be selected from iodide, bromide, chloride, fluoride, hydroxide, carbonate, bicarbonate, and any combination thereof, where the one or more anions X⁻ counterbalance one or more positive charges in the polymer. In some embodiments, one or more anions X⁻ is selected from iodide, bromide, chloride, hydroxide, and any combination thereof, where the one or more anions X⁻ counterbalance one or more positive charges in the polymer. In some embodiments, one or more anions X⁻ is selected from iodide, hydroxide, and any combination thereof, where the one or more anions X⁻ counterbalance one or more positive charges in the polymer. For example, for any of the above-described polymers including a repeating unit of Formula (XII-A), (XII-B), and/or (XII-C), the polymer can include one or more hydroxide anions, where the one or more hydroxide anions counterbalance one or more positive charges in the polymer.

The present disclosure further provides a random polymer, including (or consisting essentially of, or consisting of) repeating units of Formula (XIII-A), (XIII-B), and (XIII-C):

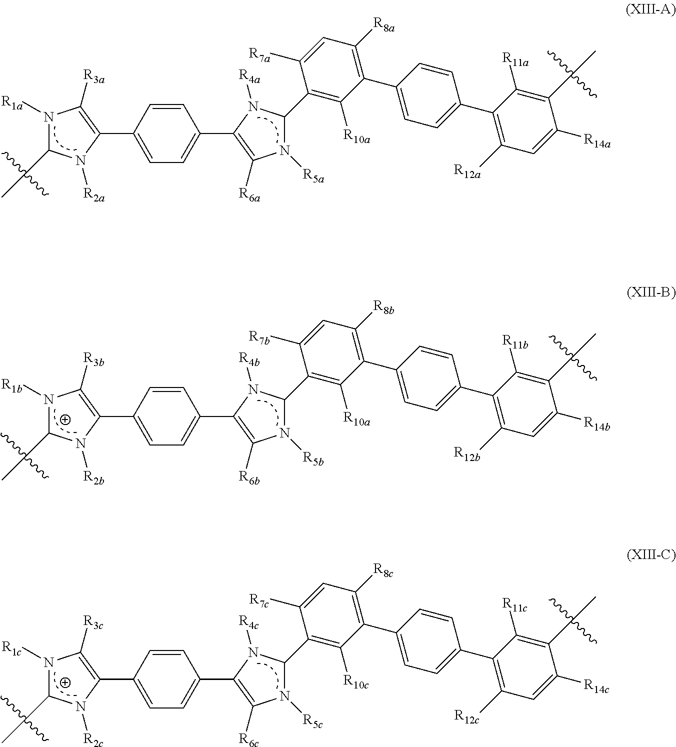

wherein
one of R₁ₐ and R₂ₐ is absent and the remaining R₁ₐ or R₂ₐ is selected from alkyl, perfluoroalkyl, heteroalkyl, aryl, and aralkyl;
one of R₁ₐ and R₅ₐ is absent and the remaining R₄ₐ or R₅ₐ is selected from alkyl, perfluoroalkyl, heteroalkyl, aryl, and aralkyl;
one of R_{1b}, R_{2b}, R_{4b}, and R_{5b} is absent and the imidazolyl group to which the absent R_{1b}, R_{2b}, R_{4b}, or R_{5b} is connected (i.e., the imidazolyl group where one of its R_{1b}, R_{2b}, R_{4b}, or R_{5b} is absent) is neutral, and the remaining three of R_{1b}, R_{2b}, R_{4b}, and R_{5b} are each independently selected from alkyl, perfluoroalkyl, heteroalkyl, aryl, and aralkyl;
R_{1c}, R_{2c}, R_{4c}, and R_{5c} are each independently selected from alkyl, perfluoroalkyl, heteroalkyl, aryl, and aralkyl;
R₃ₐ, R₆ₐ, R_{3b}, R_{6b}, R_{3c}, and R_{6c} are each independently selected from alkyl, perfluoroalkyl, heteroalkyl, aryl, aralkyl, and heteroaryl;
R₇ₐ, R₁₀ₐ, R₁₁ₐ, R₁₄ₐ, R_{7b}, R_{10b}, R_{11b}, R_{14b}, R_{7c}, R_{10c}, R_{11c}, and R_{14c} are each independently selected from H, alkyl, perfluoroalkyl, and heteroalkyl; and
R₈ₐ, R₁₂ₐ, R_{8b}, R_{12b}, R_{8c}, and R_{12c} are each independently selected from hydrogen (H), alkyl, perfluoroalkyl, and heteroalkyl;
wherein the polymer includes m mole percentage repeating units of Formula (XIII-A), n mole percentage repeating units of Formula (XIII-B), and p mole percentage repeating units of Formula (XIII-C), and
   m is from 0 mole percent to 60 mole percent,
   n+p is 40 mole percent to 100 mole percent, and
   m+n+p=100%.

In some embodiments, for the random polymer including (or consisting essentially of, or consisting of) repeating units of Formula (XIII-A), (XIII-B), and (XIII-C) described above, one of R₁ₐ and R₂ₐ is absent and the remaining R₁ₐ or R₂ₐ is selected from methyl and trifluoromethyl; and one of Ria and R₅ₐ is absent and the remaining Ra or R₅ₐ is selected from methyl and trifluoromethyl.

In some embodiments, for any of the above-described random polymers including (or consisting essentially of, or consisting of) repeating units of Formula (XIII-A), (XIII-B), and (XIII-C), one of R_{1b}, R_{2b}, R_{4b}, and R_{5b} is absent and the imidazolyl group to which the absent R_{1b}, R_{2b}, R_{4b}, or R_{5b} is connected (i.e., the imidazolyl group where one of its R_{1b}, R_{2b}, R_{4b}, or R_{5b} is absent) is neutral, and the remaining three of R_{1b}, R_{2b}, R_{4b}, and R_{5b} are each independently selected from alkyl, perfluoroalkyl, and heteroalkyl. In some embodiments, one of R_{1b}, R_{2b}, R_{4b}, and R_{5b} is absent and the imidazolyl group to which the absent R_{1b}, R_{2b}, R_{4b}, or R_{5b} is connected (i.e., the imidazolyl group where one of its Rib, R_{2b}, R_{4b}, or R_{5b} is absent) is neutral, and the remaining three of R_{1b}, R_{2b}, R_{4b}, and R_{5b} are each independently selected from methyl, and trifluoromethyl.

In some embodiments, for any of the above-described random polymers including (or consisting essentially of, or consisting of) repeating units of Formula (XIII-A), (XIII-B), and (XIII-C), R_{1c}, R_{2c}, R_{4c}, and R_{5c} are each independently selected from alkyl, perfluoroalkyl, and heteroalkyl. For example, R_{1c}, R_{2c}, R_{1c}, and R_{5c} can each be independently selected from methyl and trifluoromethyl. In some embodiments, R_{1c}, R_{2c}, R_{1c}, and R_{5c} are each independently methyl or ethyl. In some embodiments, R_{1c}, R_{2c}, R_{1c}, and R_{5c} are each methyl.

In some embodiments, for any of the above-described random polymers including (or consisting essentially of, or consisting of) repeating units of Formula (XIII-A), (XIII-B), and (XIII-C), R₃ₐ, R₆ₐ, R_{3b}, Rob, R_{3c}, and R_{6c} are each independently aryl. For example, R₃ₐ, R₆ₐ, R_{3b}, Rob, R_{3c}, and R_{6c} can each independently be phenyl.

In some embodiments, for any of the above-described random polymers including (or consisting essentially of, or consisting of) repeating units of Formula (XIII-A), (XIII-B), and (XIII-C), R₇ₐ, R₁₀ₐ, R₁₁ₐ, R₁₄ₐ, R_{7b}, R_{10b}, R_{11b}, R_{14b}, R_{7c}, R_{10c}, R_{11c}, and R_{14c} are each independently alkyl. For example, R₇ₐ, R₁₀ₐ, R₁₁ₐ, R₁₄ₐ, R_{7b}, R_{10b}, R_{11b}, R_{14b}, R_{7c}, R_{10c}, R_{11c}, and R_{14c} can each independently be methyl or ethyl. For example, R₇ₐ, R₁₀ₐ, R₁₁ₐ, R₁₄ₐ, R_{7b}, R_{10b}, R_{11b}, R_{14b}, R_{7c}, R_{10c}, R_{11c}, and R_{14c} can each independently be methyl.

In some embodiments, for any of the above-described random polymers including (or consisting essentially of, or consisting of) repeating units of Formula (XIII-A), (XIII-B), and (XIII-C), R₈ₐ, R₁₂ₐ, R_{8b}, R_{12b}, R_{8c}, and R_{12c} are each independently alkyl. For example, R₈ₐ, R₁₂ₐ, R_{8b}, R_{12b}, R_{8c}, and R_{12c} can each be independently methyl or ethyl. For example, R₈ₐ, R₁₂ₐ, R_{8b}, R_{12b}, R_{8c}, and R_{12c} can each be independently methyl.

In some embodiments, for any of the above-described random polymers including (or consisting essentially of, or consisting of) repeating units of Formula (XIII-A), (XIII-B), and (XIII-C), n and p are each more than 0 percent.

In some embodiments, for any of the above-described random polymers including (or consisting essentially of, or consisting of) repeating units of Formula (XIII-A), (XIII-B), and (XIII-C), the polymer includes one or more counterions selected from the group consisting of iodide, triiodide, hydroxide, chloride, bromide, fluoride, cyanide, acetate, carbonate, nitrate, sulfate, phosphate, triflate, tosylate, bisulfate, bicarbonate, hydrogen phosphate, and dihydrogen phosphate. In some embodiments, for any of the above-described random polymers including (or consisting essentially of, or consisting of) repeating units of Formula (XIII-A), (XIII-B), and (XIII-C), the polymer includes one or more anions X⁻ selected from iodide, bromide, chloride, fluoride, triiodide, hydroxide, carbonate, bicarbonate, cyanide, acetate, nitrate, sulfate, phosphate, triflate, tosylate, tetrakis(3,5-bis(trifluoromethyl) phenyl) borate, bis(trifluoromethane) sulfonamide, and any combination thereof, where the one or more anions X⁻ counterbalance one or more positive charges in the polymer. For example, the one or more anions X-can be selected from iodide, bromide, chloride, fluoride, hydroxide, carbonate, bicarbonate, and any combination thereof, where the one or more anions X⁻ counterbalance one or more positive charges in the polymer. For example, one or more anions X-can be selected from iodide, bromide, chloride, hydroxide, and any combination thereof, where the one or more anions X⁻ counterbalance one or more positive charges in the polymer. As another example, one or more anions X-can be selected from iodide, hydroxide, and any combination thereof, where the one or more anions X⁻ counterbalance one or more positive charges in the polymer. In some embodiments, for any of the above-mentioned random copolymers including repeating units of Formula (IV-A), (IV-B), and (IV-C), the polymer includes one or more hydroxide anions, where the one or more hydroxide anions counterbalance one or more positive charges in the polymer.

In some embodiments, the polymer includes a repeating unit including a moiety of Formula (XIV):

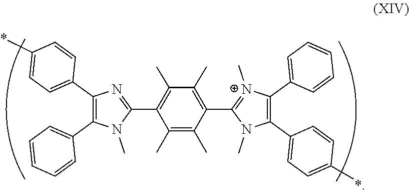

In some embodiments, the polymer includes a repeating unit including a moiety of Formula (XV):

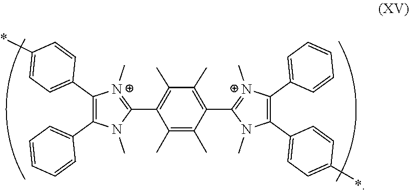

In some embodiments, the polymer includes a repeating unit including a moiety of Formula (XVI):

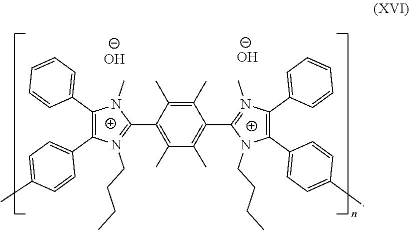

Antimicrobial polymer coatings are described in the Examples below.

### Examples

### Example 1. Antimicrobial Activity Assessment

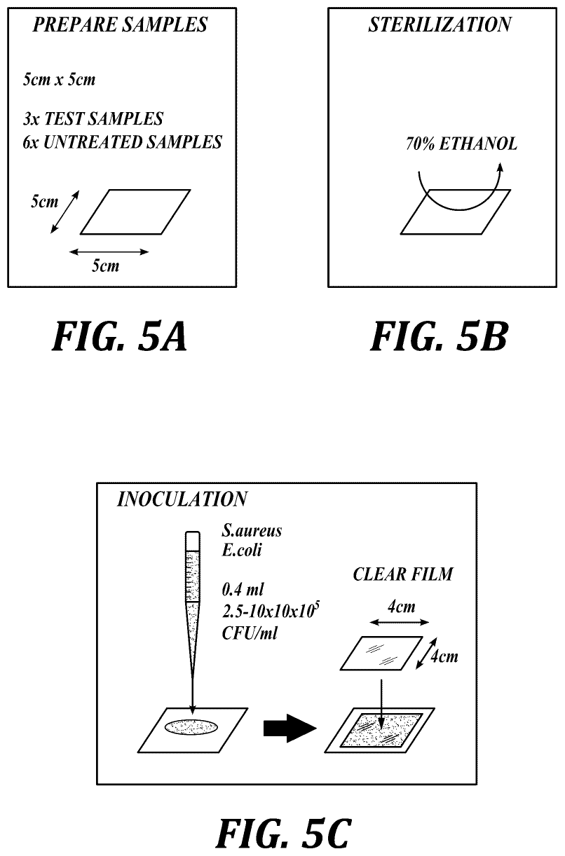

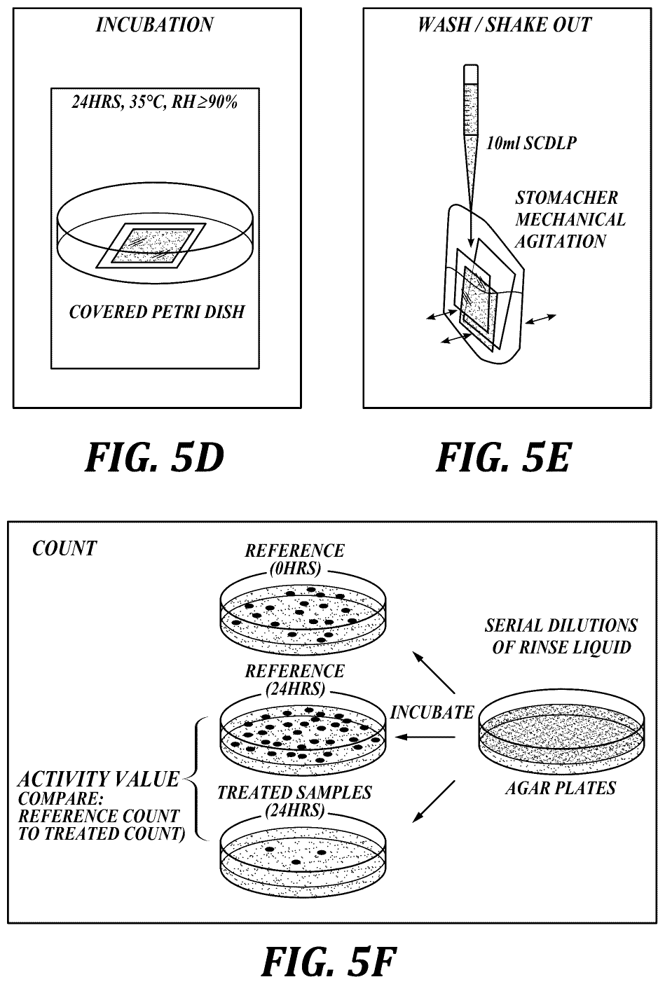

The overall antimicrobial test procedure is shown in FIG. 5. The present Example was conducted based on two different lots of polymers with different degrees of methylation (dm). The structures of the polymers are shown in Schemes 1 and 2 below. These are hereafter noted as high (91%) and low (57%) dm. Polymers in the iodide form were dissolved at 2 wt % in 50:50 (v/v) methanol: acetone by heating at 40° C. with stirring and then precipitated respectively in a 1 M aqueous solution of the desired anion: i.e., sodium chloride, sodium nitrate and ammonium acetate. The precipitated, anion-exchanged polymers were filtered, washed with water and dried (80° C., 12 h).

Solutions for casting were done by dissolving with stirring at 40° C. The high dm polymer was dissolved in pure methanol at 0.7 wt % while the low dm polymer was dissolved in 88:12 (v/v) methanol: acetone. Untreated microscope slides (AmScope BS-50P, clear glass, ground edge, 1″×3″) were cleaned prior to use by ultrasonication in methanol at room temperature for 30 min. A 1 mL aliquot of a given polymer solution was drop-cast onto the glass slide and dried at room temperature in the fume hood overnight.

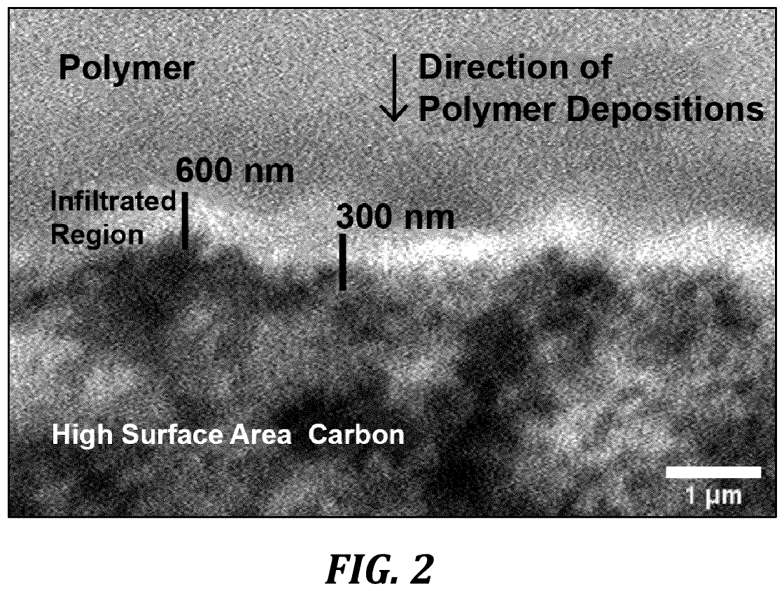

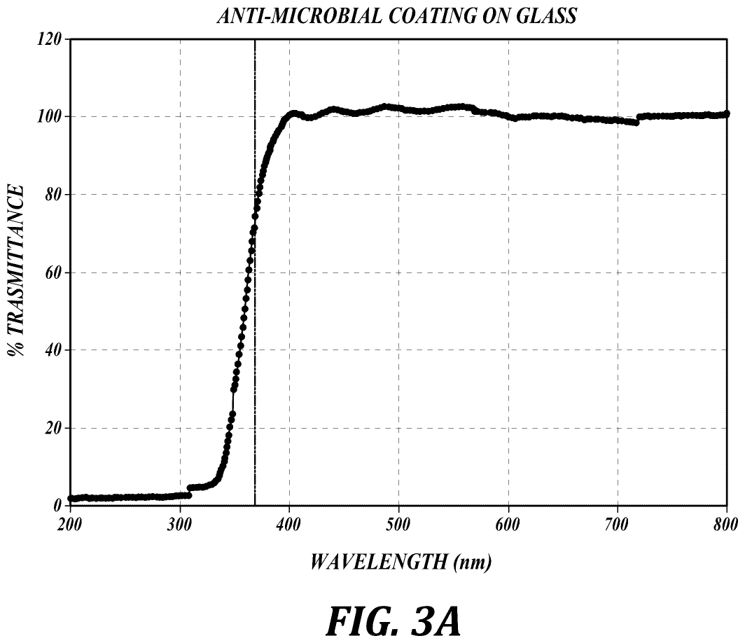

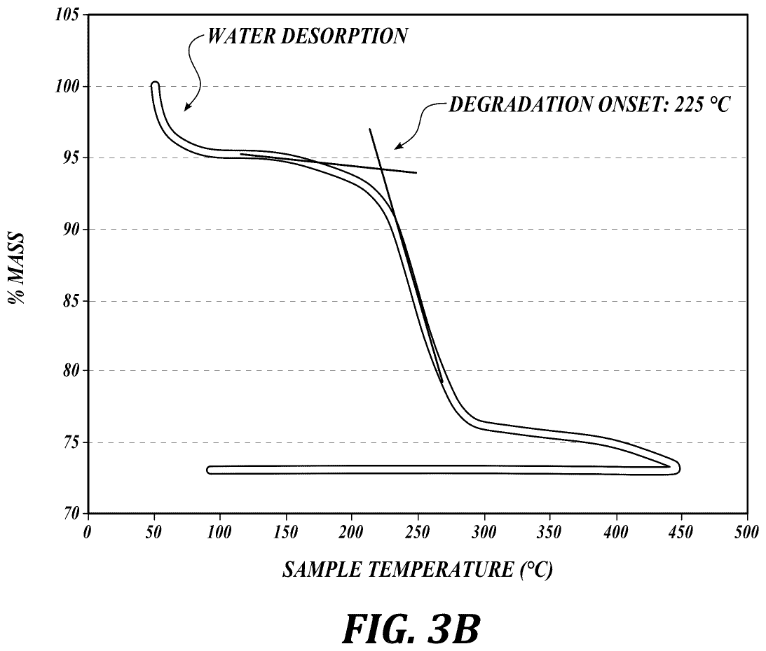

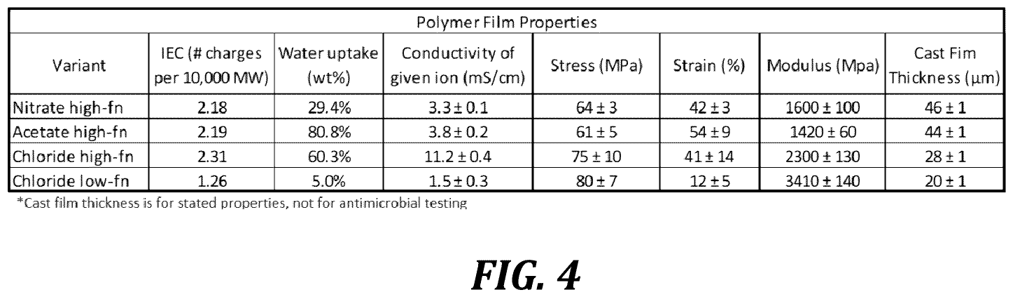

Referring to FIGS. 2 (micrograph), 3A (transmission), and 3B (TGA), the polymer-coated samples (high dm) were smooth, clear, and transparent. The polymer film properties are also shown in FIG. 4.

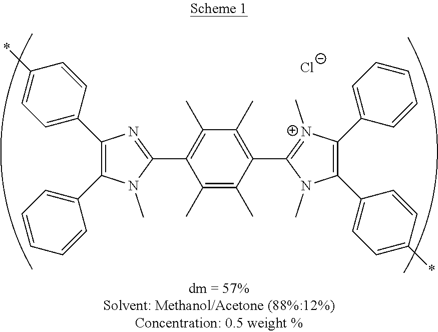

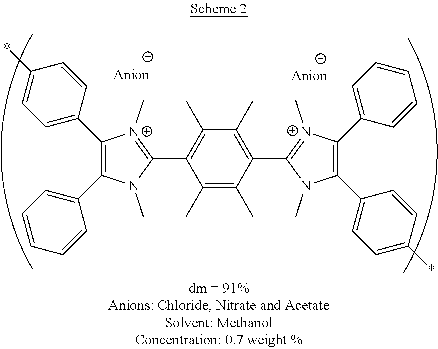

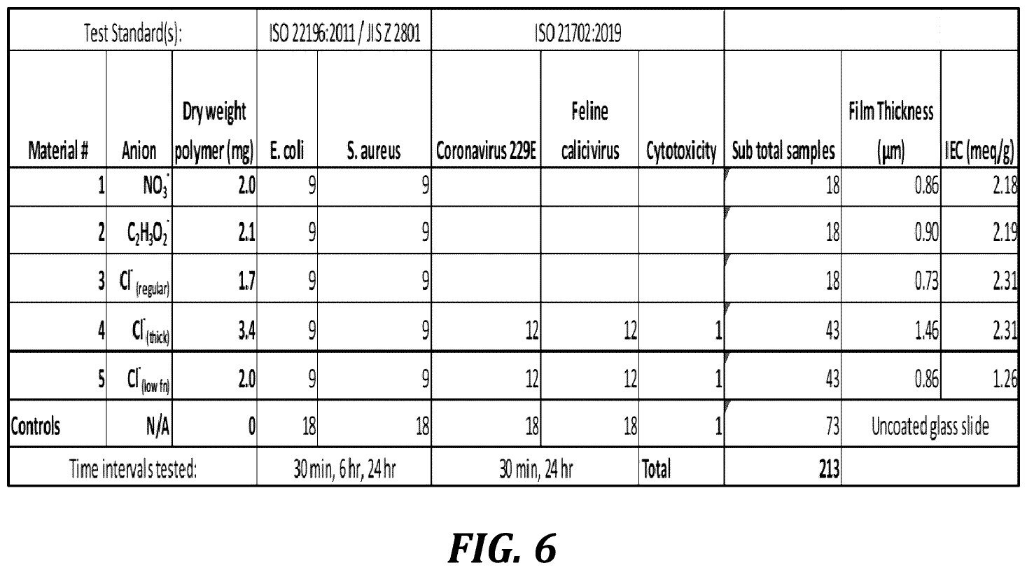

Referring to FIG. 6 and Table 1 below, Samples 1, 2, 3, 4, 5, indicate polymer anions nitrate, acetate, chloride (cast thinly), chloride, chloride (low degree of methylation) respectively for the polymers. Two controls were also included; sample C comprised of uncoated glass and SBSC was the untreated internal control provided by Situ-Bioscience. Samples were provided in triplicate.

TABLE 1
Sample compositions.

| | | Dry Film Thickness | IEC |
|---|---|---|---|
| Sample | Anion | (μm) | (meq/g) |
| 1 | NO₃⁻ | 0.9 | 2.2 |
| 2 | C₂H₃O₂⁻ | 0.9 | 2.2 |
| 3 | Cl⁻_{(regular)} | 0.7 | 2.3 |
| 4 | Cl⁻_{(thick)} | 1.5 | 2.3 |
| 5 | Cl⁻_{(low fn)} | 0.9 | 1.3 |

| | | |
|---|---|---|
| Controls | N/A | Uncoated glass slide |

### Anti-Bacterial Testing

ISO-22196 test method is designed to measure the antimicrobial properties of solid or hard surface treated test samples incubated with selected microorganisms. The basis of the test method is the incubation of the bacterial inoculum in contact with the test sample for a duration of 24 hours without drying of the inoculum. Following this exposure, the inoculated bacteria are recovered and the concentration of the organisms is determined. The antimicrobial performance is determined by comparison of the recovered organisms from the untreated material and treated material after the 24-hour incubation.

The antimicrobial performance is reported as both the Log 10 and % Reduction relative to the untreated control sample. Three timepoints were tested: 30 min, 6 hrs and 24 hrs.

### Results

Samples were all tested in triplicate to JIS Z 2801 and inoculated with S. aureus (6538) and E. coli (8739). All samples were plated twice and tested for activity at 0.5, 6 and 24 hours.

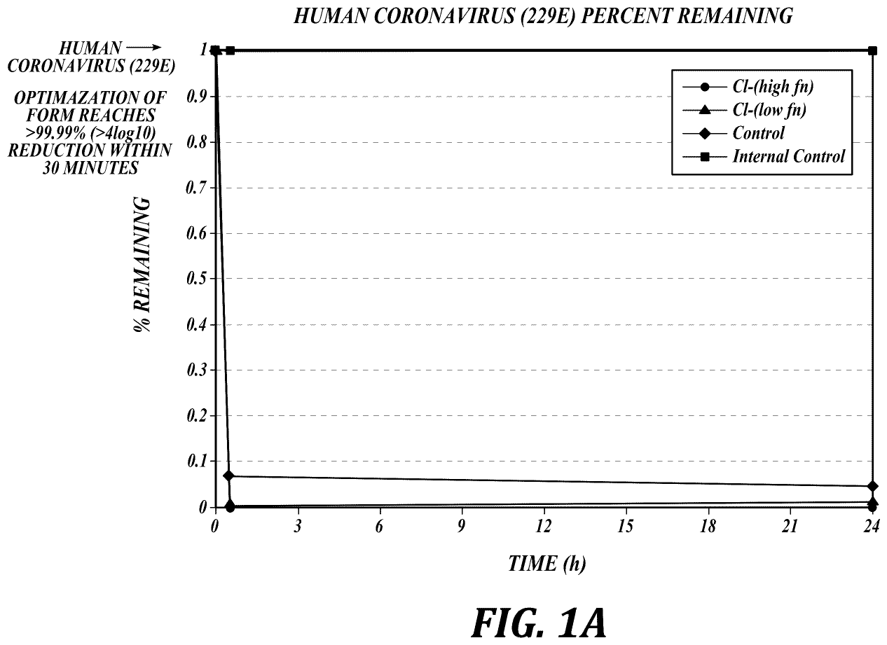

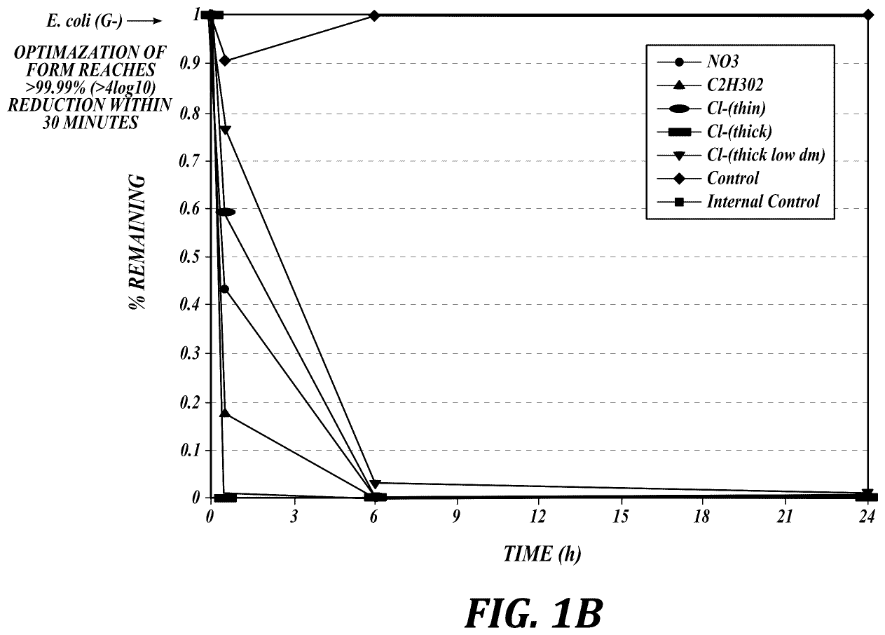

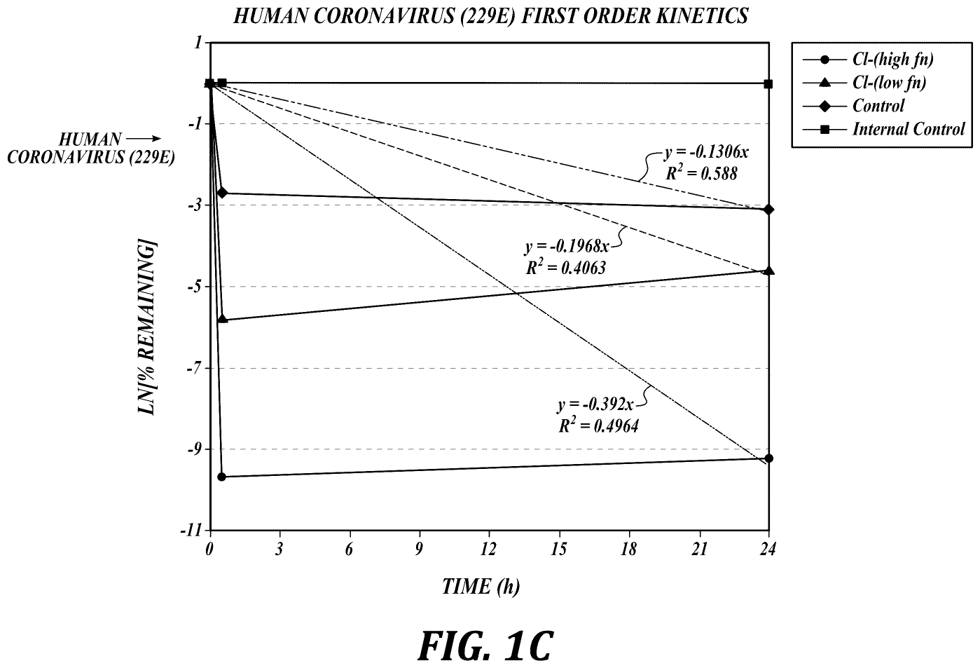

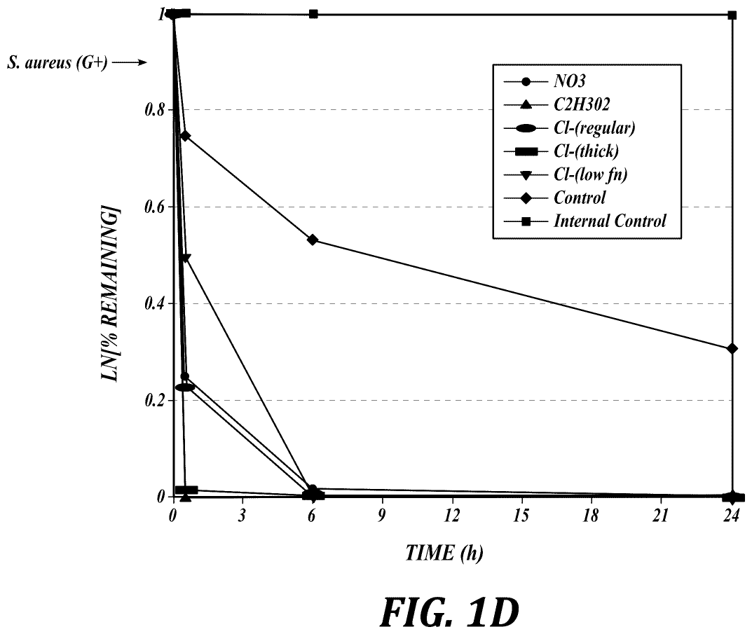

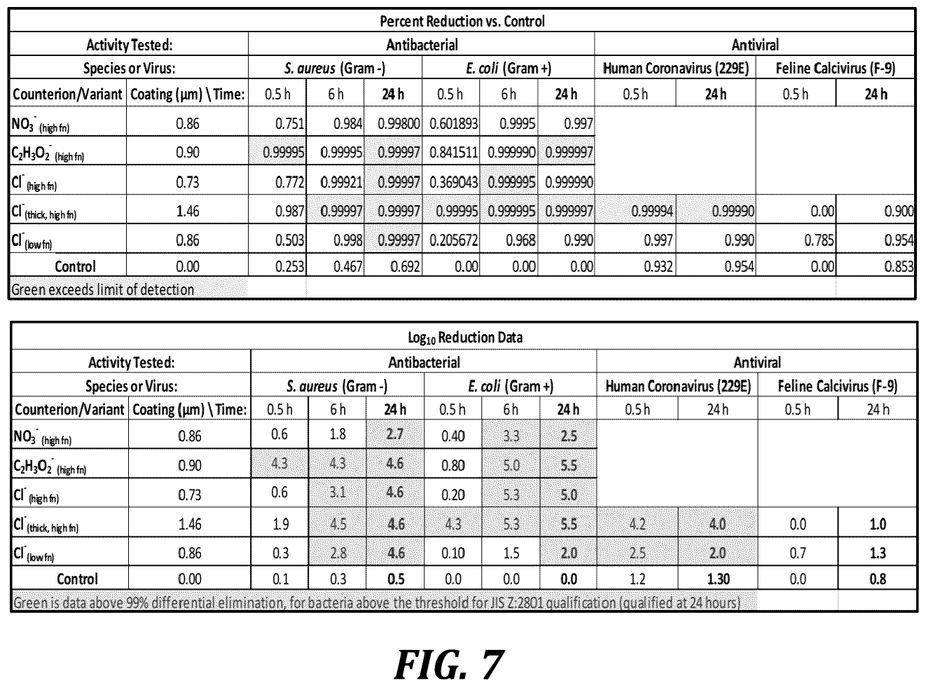

Referring to FIGS. 1B, 1D, and 7, Sample 1 showed a reduction in activity of 75.07% (0.6 Log 10 reduction), 98.42% (1.8 Log 10 reduction) and 99.80% (2.7 Log 10 reduction) to S. aureus and 56.59% (0.4 Log 10 reduction), 99.95% (3.3 Log 10 reduction) and 99.68% (2.5 Log 10 reduction) to E. coli.

Sample 2 showed a reduction in activity of 99.00% (4.3 Log 10 reduction), 99.99% (4.3 Log 10 reduction) and 99.99% (2.74.6 Log 10 reduction) to S. aureus and 82.46% (0.8 Log 10 reduction), >99.99% (5 Log 10 reduction) and >99.99% (5.5 Log 10 reduction) to E. coli.

Sample 3 showed a reduction in activity of 77.26% (0.6 Log 10 reduction), 99.92% (3.1 Log 10 reduction) and >99.99% (4.6 Log 10 reduction) to S. aureus and 40.50% (0.2 Log 10 reduction), >99.99% (5.3 Log 10 reduction) and >99.99% (5 Log 10 reduction) to E. coli.

Sample 4 showed a reduction in activity of 98.72% (1.9 Log 10 reduction), 99.99% (4.5 Log 10 reduction) and >99.99% (4.6 Log 10 reduction) to S. aureus and >99.99% (4.3 Log 10 reduction), >99.99% (5.3 Log 10 reduction) and >99.99% (5.5 Log 10 reduction) for E. coli.

Sample 5 showed a reduction in activity of 50.53% (0.3 Log 10 reduction), 99.83% (2.8 Log 10 reduction) and >99.99% (4.6 Log 10 reduction) to S. aureus and 23.42% (0.1 Log 10 reduction), 97.03% (1.5 Log 10 reduction) and 98.93% (2 Log 10 reduction) to E. coli.

Sample C showed a reduction in activity of 25.28% (0.1 Log 10 reduction), 46.68% (0.3 Log 10 reduction) and 69.24% (0.5 Log 10 reduction) to S. aureus and 9.58% (0 Log 10 reduction), 0% (0 Log 10 reduction) and 0% (0 Log 10 reduction) for E. coli.

The internal control sample SBSC Untreated Control showed bacterial concentration of 7.1E5 CFU/mL, 1.0E6 CFU/mL, at 0 and 0.5 hours, respectively, and 7.7E5 CFU/mL, 1.4E6 CFU/mL, and 2.0E6 CFU/mL at 0, 0.5 and 24 hours, respectively, for S. aureus.

The SBSC Untreated Control showed bacterial concentration of 9.3E5 CFU/mL, 1.0E6 CFU/mL for 0 and 0.5 hours, respectively, and 7.0E5 CFU/mL, 1.1E7 CFU/mL, and 1.4E7 CFU/mL at 0, 6 and 24 hours, respectively, for E. coli.

### Anti-Viral Testing

ISO 21702 provides a test method for the quantitative evaluation of virucidal activity on plastics and other non-porous surfaces. Products tested are intended to be treated antiviral products, that are tested against the specified virus. The basis of the test method is the incubation of the viral inoculum in contact with the test sample for a duration of 24 hours without drying of the inoculum. Following this exposure, the inoculated virus is recovered, and the concentration of the infective virus is determined. The antiviral performance is determined by a comparison of the recovered virus from the untreated material and treated material after the 24-hour incubation.

The antimicrobial performance is reported as both the Log 10 and % Reduction relative to the untreated control sample.

Testing was conducted with soiling by incorporation of 5% serum to the viral inoculation solution.

### Results and Discussion

Result: Log Reduction rounded to nearest tenths (ex 3.1)

Samples were all tested to ISO 21702 and inoculated with Feline Calicivirus (F-9) and Human Coronavirus (229E). All samples were tested for activity at 0.5 and 24 hours.

Cytotoxicity optimization was performed for sample 4 only

Referring to FIGS. 1A, 1C, and 7, Sample 4 showed a reduction in activity of 0% (0 Log 10 reduction), and 90% (1 Log 10 reduction) to Feline Calicivirus (F-9) and >=99.99% (4.2 Log 10 reduction), and 99.99% (4 Log 10 reduction) to Human Coronavirus (229E) at 0.5 and 24 hours respectively.

Sample 5 showed a reduction in activity of 78.5% (0.7 Log 10 reduction), and 95.4% (1.3 Log 10 reduction) to Feline Calicivirus (F-9) and 99.7% (2.5 Log 10 reduction), and 99% (2 Log 10 reduction) to Human Coronavirus (229E) at 0.5 and 24 hours respectively.

Sample C showed a reduction in activity of 0% (0 Log 10 reduction), and 85.3% (0.8 Log 10 reduction) to Feline Calicivirus (F-9) and 93.2% (1.2 Log 10 reduction), and 95.4% (1.3 Log 10 reduction) to Human Coronavirus (229E) at 0.5 and 24 hours respectively.

The internal control sample SBSC Untreated Control showed Virus concentration of 2.9E6 log 10 TCID50/sq cm, 2.9E6 log 10 TCID50/sq cm, and 6.1E6 log 10 TCID50/sq cm at 0, 0.5 and 24 hours, respectively, for Feline Calicivirus (F-9) and 6.1E5 log 10 TCID50/sq cm, 9.0E5 TCID50/sq cm and 6.2E5 Log 10 TCID50/sq cm at 0, 0.5 and 24 hours, respectively, for Human Coronavirus (229E).

ISO 21702 Inherent Cytotoxicity Optimization for sample 4 was negative.

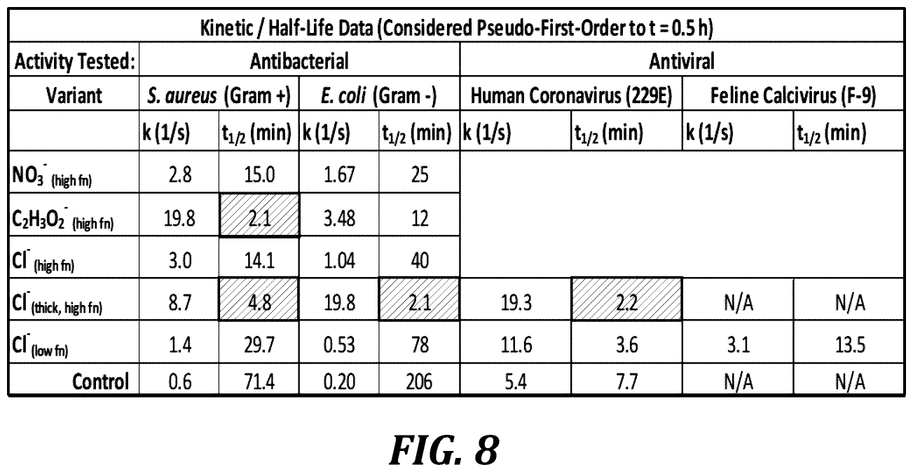

Kinetic analysis and half-life data was also obtained. The results are shown in FIG. 8.

### Detailed Antibacterial and Antiviral Tests and Results.

### ISO 22196: Antibacterial Test

A method of evaluating the antibacterial activity of antibacterial-treated plastic products including intermediate products). It is not intended to be used to evaluate the effects and propagation of bacteria on plastics without antibacterial treatments.

Antimicrobial activity is determined in the following manner:

$$R = ( Ut - U 0 ) - ( At - U 0 ) = Ut - At$$

Where

R is the antibacterial activity;

U0 is the average of the common logarithm of the number of viable bacteria, in cells/cm2, recovered from the untreated test specimens immediately after inoculation;
Ut is the average of the common logarithm of the number of viable bacteria, in cells/cm2, recovered from the untreated test specimens after 24 h
At is the average of the common logarithm of the number of viable bacteria, in cells/cm2, recovered from the treated test specimens after 24 h.

For purposes of common reference, the % reduction is also reported in the notes section for each sample result.

### Legend

CFU=colony forming unit (typically cited per unit volume or surface area). CFU is determined by bacterial plating of the test samples according to the specified method, followed by counting of the resultant colonies.

Untreated Control (UTC)-untreated control sample material used to demonstrate normal test performance, showing robust microorganism growth.

Interval—represents the point or time point from which the result value was determine; TO indicates that the result is from the soonest possible time from inoculation to recovery of the inoculated sample (typically <5 min).

Result—the result is the measure of change or abundance. Result units indicate the actual measurements, frequently relative to a control value depending on the method or test requirements.

### Notation of Changes to the Published Test Method:

Several references are made to ‘Plate count agar’ for the test method plating following neutralization and recovery of the bacterial from the test samples. As standard practice, counts are performed on appropriate plate media such as Nutrient agar, tryptic soy agar, or as required by the specific organism tested.

The published standard refers to incubation conditions of 35±1° C. Standard microbiological practice with other international methods is for incubations to occur at 37±1° C. The test conditions performed will be conducted at 37±1° C. unless specified for other temperature conditions.

Expanded Uncertainty for the Test Method of k=2 is for a 95% Confidence of Log 10 (0.136).

Uncertainty Values in CFU are obtained by converting CFU counts (C1) to Log 10 values (Log 10 C1); multiply this result by the Expanded Uncertainty (Log 10 C1*EU), then add and subtract from the Log 10 value (Log C1±(Log 10 C1*EU)); convert back by taking the anti-log (1×10∧(Log C1±Log 10 C1*EU)) which provides the upper and lower limits of 95% confidence in CFU.

### Stated Method Standard

For ISO 22196, an interlaboratory test demonstrated a results with Standard deviation of Log 10 (0.45) or ˜1/2 log. This equates to a-mean value±50% reduction.

### Conditions for a Valid Test

When the three conditions are satisfied, the test is deemed valid. If all conditions are not met, the test is not considered valid and the specimens shall be retested.

The logarithmic value of the number of viable bacteria recovered immediately after inoculation from the untreated test specimens shall satisfy the following requirement:

(Lmax−Lmin)/(Lmean)<=0.2

Where:

Lmax is the common logarithm (i.e., base 10 logarithm) of the maximum number of viable bacteria found on a specimen

Lmin is the common logarithm of the minimum number of viable bacteria found on a specimen;

Lmean is the common logarithm of the mean number of viable bacteria found on the specimens.

### Abstract

The ISO-22196 test method is designed to measure the antimicrobial properties of solid or hard surface treated test samples incubated with selected microorganisms. The basis of the test method is the incubation of the bacterial inoculum in contact with the test sample for a duration of 24 hours without drying of the inoculum. Following this exposure, the inoculated bacteria are recovered and the concentration of the organisms is determined. The antimicrobial performance is determined by comparison of the recovered organisms from the untreated material and treated material after the 24 hour incubation.

The antimicrobial performance is reported as both the Log 10 and % Reduction relative to the untreated control sample.

Three timepoints were tested: 30 min, 6 hrs and 24 hrs.

### Results and Discussion

### Results are Provided in the Result Data Tables

### Test Sample Result

Timepoint of the result: (typically TO or other time in test)

Timepoint Units: typically “hr” hours

Result: Log Reduction rounded to nearest tenths (ex 3.1)

UNITS: Log Reduction

Limit of detection (LOD): LOD is entered in the result note for each sample where no bacteria were recovered.

JIS Z 2801-Antibacterial products-Test for antimicrobial activity and efficacy

JIS Z 2801 specifies a method of evaluating the antibacterial activity of plastic products. It is a method commonly used for evaluating the antimicrobial properties of many material types that can range from coated surfaces or those of monolithic composition. The basis of the test method is the incubation of the microorganism inoculum in intimate contact with the test substance on a flat horizontal surface for 24 hours. Following this exposure, a sample of the inoculum is recovered and the concentration of the organisms is determined. The antimicrobial performance is determined by comparison of the recovered organisms from the untreated inoculum-only test substances after the selected time points and treated test substance after the selected time points. It is not intended to be used to evaluate the effects and propagation of bacteria on plastics without antibacterial treatments.

### Testing

### Inoculum Preparation

Following an overnight incubation of the test bacteria, a transfer to the inoculation solution is performed. Using a sterile inoculating loop, transfer one loop of the test bacteria into a small amount of 1/500 NB prepared. A dilution of this suspension with 1/500 NB is created as appropriate to establish an estimated bacterial concentration, to obtain a bacterial concentration that is between SES CFU/mL and 2E6 CFU/mL, with a target concentration of 1E6 CFU/mL.

### Inoculation of Test Specimens

The surface to be tested is the exposed outer surface of the product.
Pipette 0.2 mL of the test inoculum prepared onto the test surface.
Cover the test inoculum with a piece of film that measures 40 mm×40 mm and gently press down.
on the film so that the test inoculum spreads to the edges.
Make sure that the test inoculum does not leak beyond the edges of the film.
After the specimen has been inoculated and the cover film applied, replace the lid of the Petri dish.

### Incubation of the Inoculated Test Specimens

The standard procedure for incubation of the inoculated specimens is to incubate the Petri dishes containing the inoculated test specimens (including half of the untreated test specimens) at a temperature of (37±1° C.) and a relative humidity of not less than 90% for (24±1) h, unless otherwise noted.

Recovery of Bacteria from Test Specimens.

Immediately after inoculation, process half of the untreated test specimens by adding 5 mL of a suitable neutralizer to the Petri dish containing the test specimen.

Test Specimens after Incubation

After the incubation, process the remaining test specimens. Proceed immediately to count the viable bacteria recovered from the test specimen.

### Reagents

Nutrient Broth 2 (NB2)
D/E Neutralizing Broth (D/E)
Phosphate Buffered Saline (PBS)
Tryptic Soy Agar
Laboratory RO water, deionized

### Equipment List

Thermo Orbital Shaker Incubator
Scales (Mettler H80, Mettler PM-11K, Mettler MS104S/03)
Autoplater Spiral Biotech 4000
National Water Incubator
Shimadzu Spectrophotometer
MarketForge Autoclave
Hach pH Meter/02 measure/conductivity meter
Dwyer Hygrometer
Gilson Pipetters

### Test Organisms

Staphylococcus aureus
Escherichia coli

### Sample Preparations

Each test substance was prepared according to the analytic method requirements. Each test sample is prepared in triplicate unless otherwise specified. As available, flat samples are procured, and cut into a piece SO×SO mm. Sample variability are accommodated at needed for the standard test, notes regarding differences in the sample characteristic are recorded in the report summary.

TABLE 2
Antimicrobial test results
Test Method JIS Z 2801-Antibacterial products-Test for
antimicrobial activity and efficacy
Sample # 1 1

| [symbol] | Interval | Result |
|---|---|---|
| 75.07% reduction | 0.5 hr | 0.6 log10 Reduction |
| 98.42% reduction | 6 hr | 1.8 log10 Reduction |
| 99.80% reduction | 24 hr | 2.7 log10 Reduction |
| [symbol] | | |
| 58.59% reduction | 0.5 hr | 0.4 log10 Reduction |
| 99.85% reduction | 6 hr | 3.3 log10 Reduction |
| 99.68% reduction | 24 hr | 2.8 log10 Reduction |
| variation present between replicates | | |

Sample # 2 2

| [symbol] | Interval | Result |
|---|---|---|
| >99.99% reduction, no organisms | 0.6 hr | 4.3 log10 Reduction |
| recovered: LOD > 500 CFU/mL | | |
| 99.99% reduction | 6 hr | 4.3 log10 Reduction |
| >99.99% reduction, no organisms | 24 hr | 4.8 log10 Reduction |
| recovered: LOD > 500 CFU/mL | | |
| [symbol] | | |
| 52.46% reduction | 0.6 hr | 0.8 log10 Reduction |
| >99.99% reduction | 6 hr | 6 log10 Reduction |
| >99.99% reduction, no organisms | 24 hr | 5.3 log10 Reduction |
| recovered: LOD > 500 CFU/mL | | |

Sample # 3 3

| [symbol] | Interval | Result |
|---|---|---|
| 77.26% reduction | 0.6 hr | 0.8 log10 Reduction |
| 99.92% reduction, variation present | 6 hr | 3.1 log10 Reduction |
| between replicates | | |
| >99.99% reduction, no organisms | 24 hr | 4.6 log10 Reduction |
| recovered: LOD > 500 CFU/mL | | |

[symbol]

indicates data missing or illegible when filed

### ISO 21702: Antiviral Test

The ISO 21702 provides a test method for the quantitative evaluation of virucidal activity on plastics and other non-porous surfaces. Products tested are intended to be treated antiviral products, that are tested against the specified virus. The basis of the test method is the incubation of the viral inoculum in contact with the test sample for a duration of 24 hours without drying of the inoculum. Following this exposure, the inoculated virus is recovered, and the concentration of the infective virus is determined. The antiviral performance is determined by a comparison of the recovered virus from the untreated material and treated material after the 24-hour incubation.

The antimicrobial performance is reported as both the Log 10 and % Reduction relative to the untreated control sample.

### Results and Discussion

### Results are Provided in the Result Data Tables

Testing was conducted with soiling by incorporation of 5% serum to the viral inoculation solution.

ISO 21702 specifies a method of evaluating virucidal activity of non-porous surfaces. Each product was tested using clean test condition (no additional soil). Unless otherwise specified, secondary effects of antibacterial treatments, the measured antimicrobial performance, or the durability of a measured activity are not covered by the standard. The standard is not intended to be used or referenced as a method to document or claim antimicrobial performance unless indicated by the test report. The determinations of product performance within a given environment can vary dramatically, and must be specifically documented and then determined within the context of a specific project plan.

### Methods

### Project-Specialized Organism Culturing-Virus

Cell culturing for non-standard virus used in a given test method. For each virus, the necessary media, and culture conditions are employed according to standard requirements established by the lab.

### ISO 21702—Measurement of Antiviral Activity on Plastics and Other Non-Porous Surfaces

The ISO 21702 method is used to evaluate the virucidal efficacy of a non-porous product. Testing can incorporate different exposure times, soiling, and virus types and other variables according to the test standard or specific needs of a product. The most common test conditions employ the standard method protocol requiring a 24-hour exposure to the test material depending on the intended use of the product. Test virus are prepared in advance of the testing followed by a determination of viral titer. The inocula created is then utilized as the inocula for the exposure of the test material to the virus.

### ISO 21702 Inherent Cytotoxicity Optimization

Antiviral testing requires the use of host cells for propagation and enumeration of the viral concentration. The host cells must be viable and biologically intact to allow viral infection. Successful infection by the virus results in the replication and ultimate lysis and loss of the host cell. This process provides the means by which viruses are measured. Cytotoxicity of the host cell as a result of any chemical carry-over from the test sample can affect the biological viability of the host cell and interfere with needed processes subsequent to the exposure of the cells to the virus. This interference is generally considered as the inherent cytotoxicity of the test sample.

The test is conducted by incubating the test sample for 30 seconds with the neutralizing recovery solution, followed by exposure of the host cells to eight concentrations of the recovered solution. Inherent cytotoxicity is identified by the loss of the cell culture viability.

### Testing

### Inoculum Preparation

A known viral titer suspension is prepared to a concentration of at least 1E6 TCID50/mL. Passaged of the virus are not used beyond ten passes from the original seed culture.

### Experimental Conditions

Following inoculation, the samples are incubated at 25 C±1° C. (unless otherwise specified). The incubation is conducted to prevent the inoculum from drying while in contact with the test surface. Following the incubation period, the virus is recovered in neutralizing media and then diluted for culturing.

Recovery of Virus from Test Specimens.

Two time points are created for each test item, a washout of the inoculated sample is collected immediately after inoculation by addition of the selected neutralizer solution by placing the sample into a vial and adding 10 mL of the neutralizer, followed by vortexing.

A second recovery is created following the intended incubation time (24 hr), after which the sample is placed into a vial with 10 mL of the neutralizing solution and vortexed. Following the test sample neutralization, aliquots of the sample are recovered and used to determine the infective titer following the respective incubation periods.

### Reagents

Dulbecco's Modified Eagle Medium (DMEM; EM-1)
Soybean Casein Lecithin Polysorbate 80 Medium (SCDLP)
Phosphate Buffered Saline (PBS)
Formaldehyde solution (3.7%)
Crystal Violet (0.5%)
Fetal bovine serum
Viral Maintenance medium
Trypsin
Ethylenediaminetetraacetic acid solution (EDTA)
Laboratory RO water, deionized

### Equipment List

Thermo Orbital Shaker Incubator
Scales (Mettler H80, Mettler PM-11K, Mettler MS104S/03)
Nuaire BSL 2 cabinet
Nuaire water-jacketed incubator
Nikon inverted microscope
Vortex mixer
Centrifuge
Liquid Nitrogen Dewar
MarketForge Autoclave
Hach pH Meter/02 measure/conductivity meter
Dwyer Hygrometer
Gilson Pipettes

### ISO 21702—Measurement of Antiviral Activity on Plastics and Other Non-Porous Surfaces

Feline Calicivirus (F-9)
{CCL-94 Eu cell Host)
Human Coronavirus (229E}
Human lung fibroblast MRC-5 (CCL-171}

### Sample Preparations

Each test substance was prepared according to the analytic method requirements. Each test sample is prepared in triplicate for each time point. As available, the samples are cut into a piece approximately 50 mm×50 mm. Sample variability is accommodated as needed for the standard test: notes regarding differences in the sample characteristic are recorded in the report summary.

Ideally, the test sample is flat and non-hydrophobic and allow layering of the inoculum over the sample surface.

### Calculations

End-point dilutions are conducted with the recovered virus inocula using serial log 10 dilution factors. TCIDSO (Spearman-Karber; modified by M. A. Ramakrishnan) is used to determine the concentration of the inoculated virus based on the outcome of the end-point dilution resulting in the CTE of the host cells. It represents the end-point dilution (average) of the host cell monolayers exhibiting the CTE.

$$Log 10 \; 50 \% end - point Dilution = - [ ( total number of CTE wells / total number of dilution replicates ) + 0.5 ] \times log dilution factor \\ R = - [ Total CTE - replicate count per dilution ) + 0.5 ] \times Log dilution factor$$

R=The log 50% end-point dilution

Total CTE—is the average of the common logarithm of the number of viable virus, in cells/cm2, recovered from the untreated test specimens immediately after inoculation;

Replicate count per dilution—the numbers of well replicates inoculated at each dilution

Log dilution factor—is the dilution factor used for each serial dilution (typically 10× or log 10 (10)=1)

### Antiviral Activity Value

R=U(t24)-C(t24) R=the antiviral activity value C (t24)=the common logarithm average of 3 infectivity titer values after 24 hours from the untreated material

U(t24)=the common logarithm average of 3 infectivity titer values after the contact time (24 hr) with the treated (test) sample

### Statistical Methods

Replicate data are utilized in the calculation by the Spearman-Karber method, no additional statistical analysis is conducted.

TABLE 3
Antiviral test results
Test Method ISO 21702-Measurement of antiviral activity on
plastics and other non-porous surfaces
Sample # 1 4

| Inoculum: Felina Calicivirus (F-9) | | |
|---|---|---|
| Notes Section | Interval | Result |
| percent reduction = 0 | 30 min | 0 log10 Reduction |
| percent reduction = 90.0 | 24 hr | 1 log10 Reduction |
| Inoculum: Human Coronavirus (229E) | | |
| Notes Section | | |
| percent reduction >= 90.0 | 30 min | 4.2 log10 Reduction |
| percent reduction = 99.99 | 24 hr | 4 log10 Reduction |

Sample # 2 5

| Inoculum: Felina Calicivirus (F-9) | | |
|---|---|---|
| Notes Section | Interval | Result |
| percent reduction = 78.5 | 30 min | 0.7 log10 Reduction |
| percent reduction = 95.4 | 24 hr | 1.3 log10 Reduction |
| Inoculum: Human Coronavirus (229E) | | |
| Notes Section | | |
| percent reduction = 99.7 | 30 min | 2.5 log10 Reduction |
| percent reduction = 99 | 24 hr | 2 log10 Reduction |

Sample # 3 O

| Inoculum: Felina Calicivirus (F-9) | | |
|---|---|---|
| Notes Section | Interval | Result |
| percent reduction = 0 | 30 min | 0 log10 Reduction |
| percent reduction = 85.3 | 24 hr | 0.8 log10 Reduction |
| Inoculum: Human Coronavirus (229E) | | |
| Notes Section | | |
| percent reduction = 93.2 | 30 min | 1.2 log10 Reduction |
| percent reduction = 95.4 | 24 hr | 1.3 log10 Reduction |

Sample # 5 SBSC Untreated Control

| Inoculum: Felina Calicivirus (F-9) | | |
|---|---|---|
| Notes Section | Interval | Result |
| virus concentration | 0 min | 2900000Log10 [symbol] / |
| | | sq cm |
| virus concentration | 30 min | 2900000Log10 [symbol] / |
| | | sq cm |

[symbol]

indicates data missing or illegible when filed

### Example 2. Antimicrobial Activity Against Escherichia Coli

The polymer used was HMT-PMBI having 89% degree of methylation, as described, for example, in PCT publication No. WO2015/157848, incorporated herein by reference in its entirety.

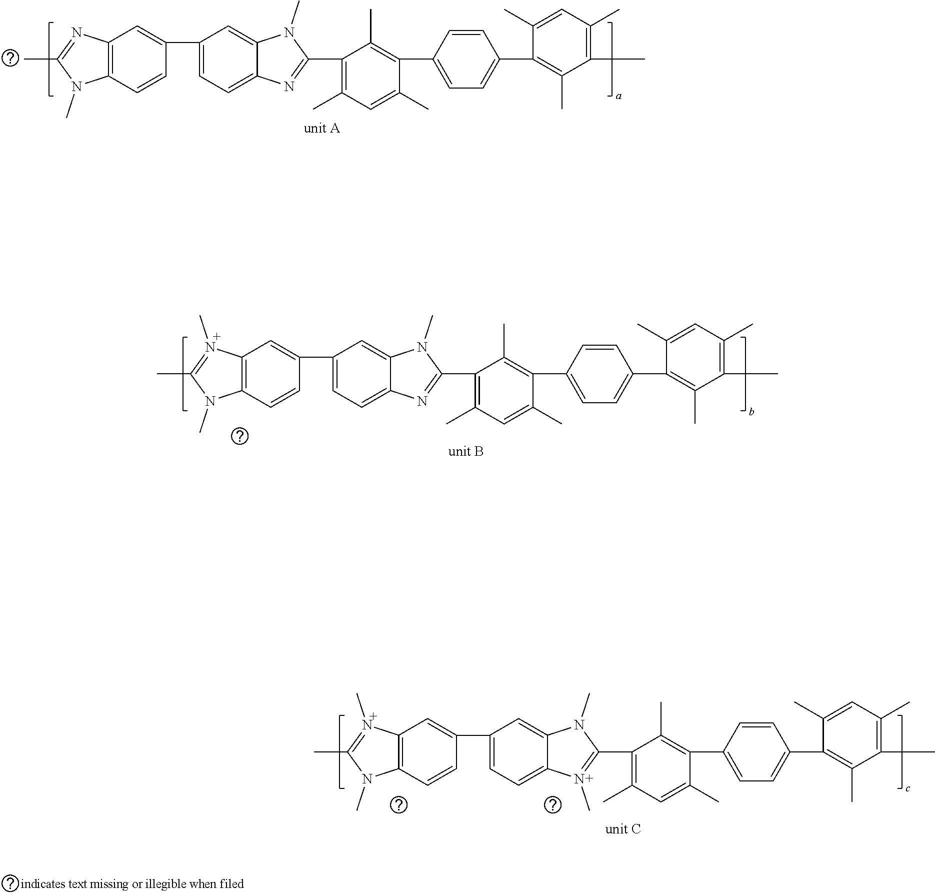

JIS Z 2801:2010, Japanese Industrial Standard Test for Antimicrobial Activity and Efficacy in Antimicrobial Products, was followed. Specifics of the test method applied to this project are described below.

This test method was designed to evaluate (quantitatively) the antimicrobial effectiveness of agent(s) incorporated or bound into or onto mainly flat (two dimensional) hydrophobic or polymeric surfaces.

The test organism was Escherichia coli ATCC #8739.

Submitted samples were inoculated with 0.4 mL of a 0.2% nutrient broth seeded with a standardized culture of the test organism in triplicate. The inoculated samples were covered with an inert film and incubated at 36±2° C. in a humidity chamber for 24 hours. Surviving microorganisms were recovered via elution of the broth inoculum from the test sample into neutralizing broth. Microbial counts of the samples were determined and the percent reduction of microorganisms (treated versus untreated samples at timepoint) was calculated.

A 1.2 mil polymeric membrane showed antimicrobial activity against Escherichia coli after 24 hours incubation in the JIS Z 2801 test method with 99.99% reduction and an antimicrobial activity value of 4.04. Percent reduction and antimicrobial activity were calculated against the control at the 24-hour timepoint.

TABLE 4
Test inoculum concentration

| | Concentration |
|---|---|
| Organism | cfu/mL |
| Escherichia coli | 1.19 × 10⁶ |
| ATCC# 8739 | |

TABLE 5
Recovered organisms from controls at Time = 0 hours

| Lab | Escherichia coli ATCC# 8739 |
|---|---|
| Identification | cells/cm² |
| Internal Control | 2.50 × 10⁴ |

TABLE 6
Recovered organisms from controls and samples at Time = 24 hours

| | Escherichia coli | | |
|---|---|---|---|
| Lab | ATCC# 8739 | % | Antimicrobial |
| Identification | cells/cm² | Reduction | Activity |
| Internal Control | 5.19 × 10⁵ | n/a | n/a |
| 12966-A | 47.8 | 99.99% | 4.04 |

### Example 3. Antimicrobial Activity Against Staph. Aureus

The polymer used was HMT-PMBI having 89% degree of methylation, as described, for example, in PCT publication No. WO2015/157848, incorporated herein by reference in its entirety.

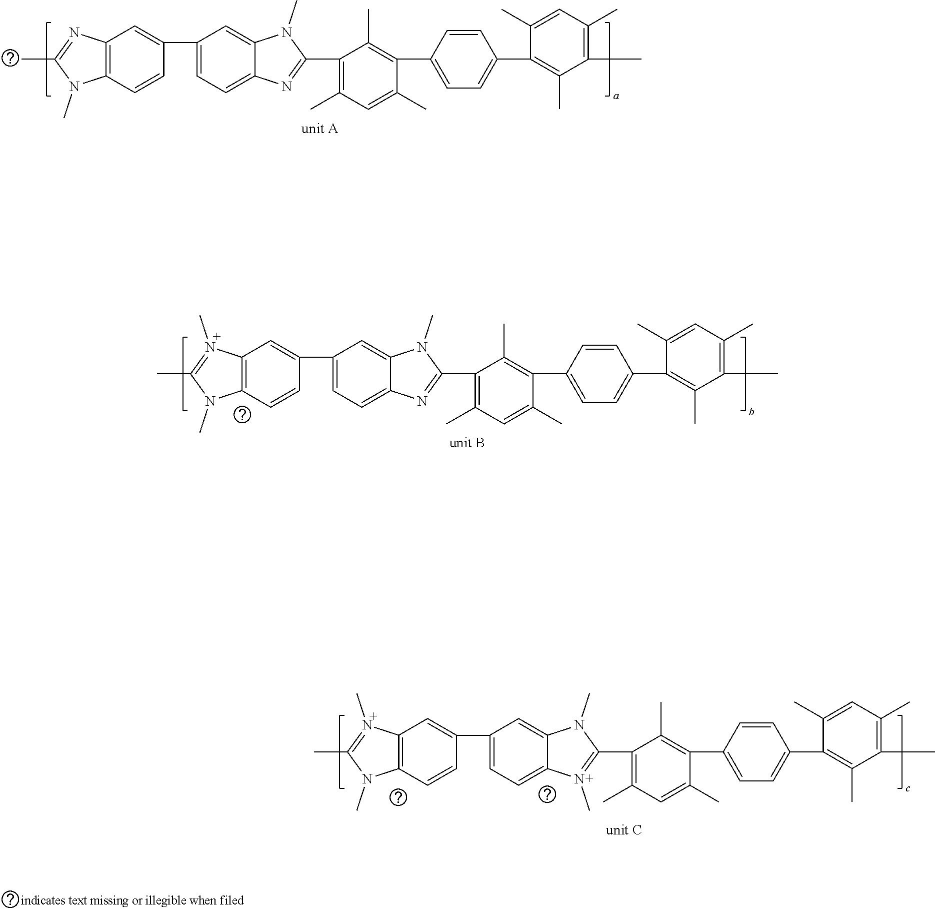

JIS Z 2801:2010, Japanese Industrial Standard Test for Antimicrobial Activity and Efficacy in Antimicrobial Products, was followed. Specifics of the test method applied to this project are described below.

This test method was designed to evaluate (quantitatively) the antimicrobial effectiveness of agent(s) incorporated or bound into or onto mainly flat (two dimensional) hydrophobic or polymeric surfaces.

The test organism was Staphylococcus aureus ATCC #6538.

Submitted samples were inoculated with 0.4 mL of a 0.2% nutrient broth seeded with a standardized culture of the test organism in triplicate. The inoculated samples were covered with an inert film and incubated at 36±2° C. in a humidity chamber for 24 hours. Surviving microorganisms were recovered via elution of the broth inoculum from the test sample into neutralizing broth. Microbial counts of the samples were determined and the percent reduction of microorganisms (treated versus untreated samples at timepoint) was calculated.

A 1.2 mil polymer membrane showed antimicrobial activity against Staphylococcus aureus after 24 hours incubation in the JIS Z 2801 test method with >99.94% reduction and an antimicrobial activity value of >3.19. Percent reduction and antimicrobial activity were calculated against the control at the 24-hour timepoint.

TABLE 7
Test inoculum concentration

| | Concentration |
|---|---|
| Organism | cfu/mL |
| Staphylococcus aureus | 1.19 × 10⁶ |
| ATCG | |
| #6536 | |

TABLE 8
Recovered organisms from controls at Time = 0 hours

| Lab | Staphylococcus aureus ATCC #6538 |
|---|---|
| Identification | cells/cm² |
| Internal Control | 1.88 × 10⁴ |

TABLE 9
Recovered organisms from controls and samples at Time = 24 hours

| | Staphylococcus aureus | | |
|---|---|---|---|
| Lab | ATCC #6538 | % | Antimicrobial |
| Identification | cells/cm² | Reduction | Activity |
| Internal Control | 5.89 × 10³ | n/a | n/a |
| 12966-B | <3.80 | >99.94% | >3.19 |

### Example 4. Antifouling Tests

Test polymeric coupons are prepared by spraying a 5 wt % methanolic solution of a partially demethylated polymer material of the present disclosure, in mixed chloride/iodide form, onto glass slides, to afford continuous films of a given thickness and a given mass loading. Coupons were then subjected to biofilm growth conditions and cell enumeration as per ASTM E2647-13, a standard test method for quantification of Pseudomonas aeruginosa biofilm grown using drip flow biofilm reactor with low shear and continuous flow, incorporated herein by reference in its entirety.

Specifically, ASTM E2647-13 specifies the operational parameters required to grow a repeatable Pseudomonas aeruginosa biofilm close to the air/liquid interface in a reactor with a continuous flow of nutrients under low fluid shear conditions. The resulting biofilm is representative of generalized situations where biofilm exists at the air/liquid interface under low fluid shear rather than representative of one particular environment.

The test method uses the drip flow reactor. The drip flow reactor (DFR) is a plug flow reactor with laminar flow resulting in low fluid shear. The reactor is versatile and can also be used for growing and/or characterizing biofilms of different species.

This test method describes how to sample and analyze biofilm for viable cells. Biofilm population density is recorded as log colony forming units per surface area.

The test method is used for growing a repeatable P. aeruginosa biofilm in a drip flow reactor. The biofilm is established by operating the reactor in batch mode (no flow of nutrients) for 6 h. A mature biofilm forms while the reactor operates for an additional 48 h with a continuous flow of nutrients. During continuous flow, the biofilm experiences very low shear caused by the gravity flow of media dripping onto a surface set at a 10° angle. At the end of the 54 h, biofilm accumulation is quantified by removing coupons from the reactor channels, rinsing the coupons to remove the planktonic cells, scraping the biofilm from the coupon surface, disaggregating the clumps, then diluting and plating for viable cell enumeration.

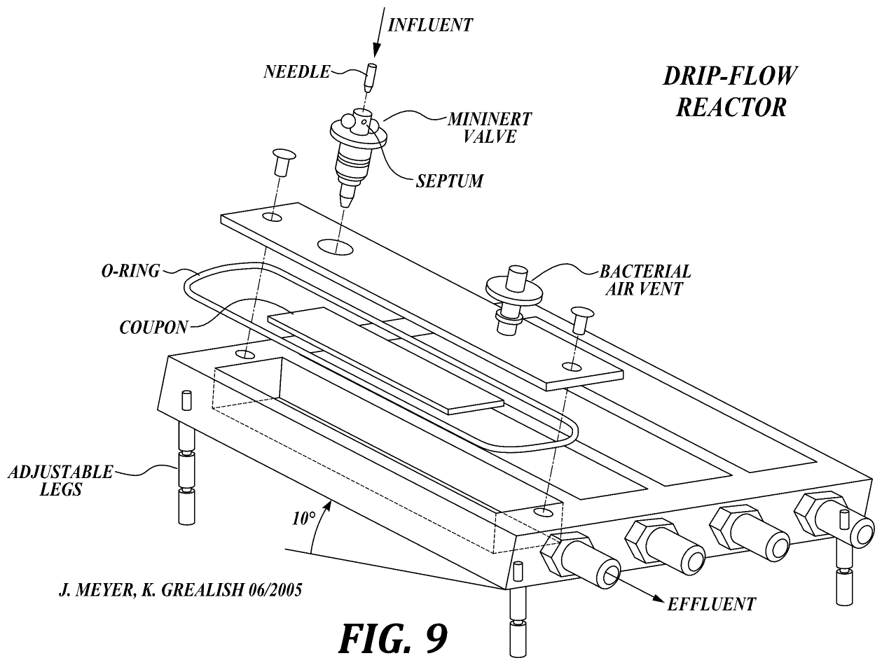

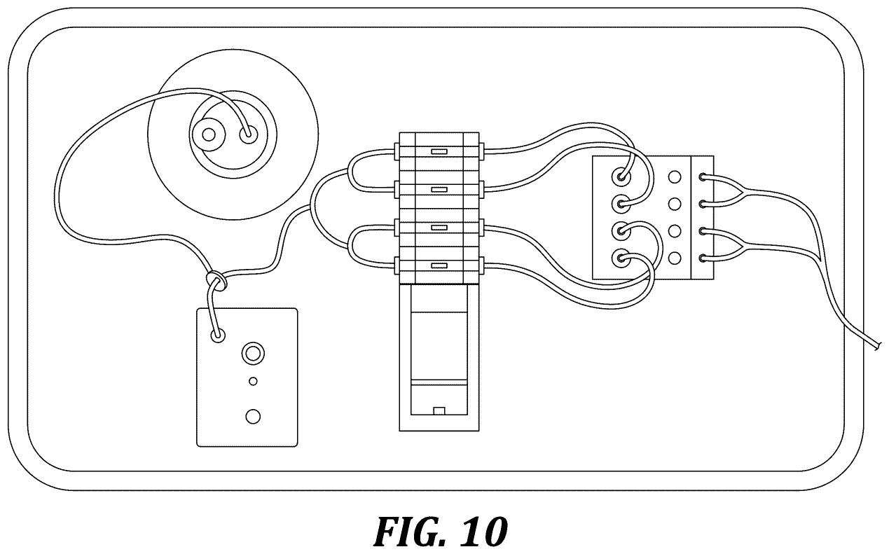

The experimental setup is as illustrated in FIGS. 9 and 10.

Purity of Water—All reference to water as diluent or reagent shall mean distilled water or water of equal purity.

### Culture Media:

Bacterial Liquid Growth Broth—Tryptic Soy Broth (TSB) 7 is recommended. Two different TSB concentrations are used in the test method, 3000 mg/L for the inoculum and batch reactor operation and 270 mg/L for the continuous flow reactor operation.

Bacterial Plating Medium—R2A agar7 is recommended.

Buffered Water—0.0425 g/L KH2PO4 distilled water, filter sterilized and 0.405 g/L MgCl·6H2O distilled water, filter-sterilized (prepared according to Method 9050 C.1a).

Pseudomonas aeruginosa (ATCC 700888) is the organism used in this test.

Aseptically remove an isolated colony from an R2A plate and inoculate into 100 mL of sterile bacterial liquid growth broth (3000 mg TSB/L). Incubate bacterial suspension in an environmental shaker at 35+2° C. for 20 to 24 h. Viable bacterial density should equal 108 CFU/mL and may be checked by serial dilution and plating.

### Procedure

### The Batch Phase:

Place the cooled reactor in a level position on the bench top. Aseptically add 15 mL of sterile 3000 mg TSB/L and 1 mL of inoculum to each channel. Tighten each channel lid securely with nylon screws. Incubate the reactor system in batch mode at room temperature (21 6 2° C.) for 6 h, in the level position. Remove foil from the effluent tubing and attach end into a waste carboy. Do not unclamp until continuous flow phase.

### Preparation for Continuous Flow Phase:

Prepare continuous flow nutrient broth by adding sterilized bacterial liquid growth medium to 20 L sterile reagent grade water so that final concentration is equal to 270 mg TSB/L (see 7.2.1). That is, dissolve and sterilize a broth concentrate in a smaller volume of water to prevent caramelization that can occur under the lengthy sterilization times required for large volumes. Aseptically pour the concentrated medium into the carboy of sterile water to make a total of 20 L.

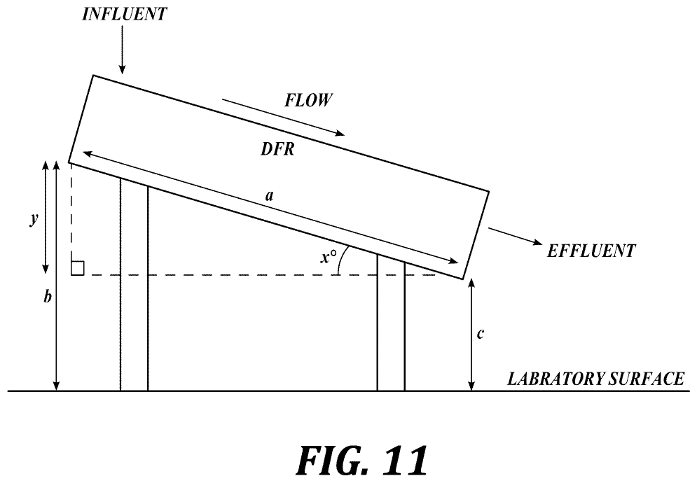

Adjust Reactor Angle (FIGURE. 11):
Measure α, the length of the reactor base, in cm.
Angle (x) is 10°.
Calculate y using the following equation:

y−a[sin (x)])
where:
α=measured length of reactor base in cm,
x=angle) (10°, and
y=difference between length of b and length of c in cm.

Decide upon the lengths of b and c to obtain the required difference (y). Use the following equation:

$$Y = b - c$$

where:
b=measured length (cm) from bottom corner of influent end of reactor base to laboratory surface,
c=measured length (cm) from bottom corner of effluent end of reactor base to laboratory surface, and
y=difference between length of b and length of c in cm.

Unclamp the effluent tubing and attach the legs to the DFR. Adjust the legs of the reactor chamber until the required lengths (b and c) are achieved so that it slopes downward 10°.

### Continuous Flow Phase:

Aseptically connect the influent nutrient tubing line to the carboy containing the continuous flow nutrient broth. Feed each line through a pump head and connect a sterile needle on the end of each line. Aseptically attach the influent tubing by inserting the sterile needle through the Mininert valves in the channel lids.

Turn on the pump, allowing media to slowly drip onto the bacterial cells attached to the coupon. A continuousflow of nutrients is pumped into the reactor through a pump set at a flow rate equal to 200 mL/h (50 mL/h per channel). The media should flow downward from the influent port to the effluent port. Periodically check the reactor for proper drainage and leaks. If problems occur, visually inspect the influent port and the tubing for bacterial plugging. The reactor is operated in CF mode for 48 h.

### Sampling the Biofilm:

Prepare sampling materials: vortex, homogenizer, sterile beakers, sterile centrifuge tubes, culture tubes, pipettes, empty sterile petri dish, sterile spatulas, and flame sterilized stainless steel hemostat or forceps.

Loosen channel lid screws with gloved hands and lift channel lid up. Aseptically remove one of the coupons by gently lifting up the coupon with sterile hemostat. Hold coupon over a sterile petri dish while carrying to the sampling area. Hold the coupon with flame sterilized hemostat being careful not to disturb the attached biofilm. Rinse the coupon to remove planktonic cells: Gently immerse coupon into the centrifuge tube containing 45 mL sterile buffered water with a fluid motion until slide is completely covered. Immediately reverse motion to remove the slide, being careful not to agitate liquid and biofilm.

Remove the biofilm from the coupon: Scrape biofilm-covered coupon surface in a downward direction for approximately 15 s, using the flat end of a sterile spatula or scraper, into the beaker containing 45 mL of sterile dilution buffer. Rinse the spatula or scraper by stirring it in the beaker. Repeat the scraping and rinsing process 3 to 4 times, ensuring full coverage of the coupon surface.

Hold the coupon at a 60° angle over the sterile beaker and pipette 1 mL of sterile buffered water over the top surface of the coupon. Repeat for a total of 5 rinses. The final volume in the beaker is 50 mL.

### Analyze the Biofilm Sample:

Homogenize the scraped biofilm sample in the beaker at 20,500±5000 r/min for 30s. If more than one biofilm sample is taken, rinse the homogenizer probe between each new sample as follows: Homogenize a dilution blank for 30s at 20,500±5000 r/min, homogenize a tube containing 70% ethanol for 15 s, then remove the probe and let the probe sit in the ethanol tube for 1 min. Shake any remaining ethanol off the probe, reattach probe, and homogenize a dilution blank for 30s. Homogenize a third dilution blank and then homogenize the next sample beaker. Homogenizing the sample disaggregates the biofilm clumps to form a homogeneous cell suspension. Improper disaggregation could result in an underestimation of the viable cells present in the sample.

Perform serial 10-fold dilutions on the sample using sterile culture tubes. Plate each dilution in duplicate for colony growth using an accepted plating technique such as spread or spiral plating (Practice D5465). Incubate the plates for 17 to 20 h at 35±2° C.

### Cell Enumeration:

Count the appropriate number of colonies according to the plating method used. Calculate the arithmetic mean of the colonies counted on the duplicate plates. The log density for one coupon is calculated as follows:

$${LOG}_{10} ( CFU / {cm}^{2} ) = {LOG}_{10} [ ( X / B ) ( V / A ) ( D ) ]$$

where:
X=mean CFU,
B=volume plated,
V=volume scraped into,
A=surface area scraped, and
D=dilution.

Calculate the overall biofilm accumulation by calculating the mean of the log densities.

### Example 4. Performance of Additional Polymer

Another exemplary polymer, having the form shown in Scheme 3, was tested.

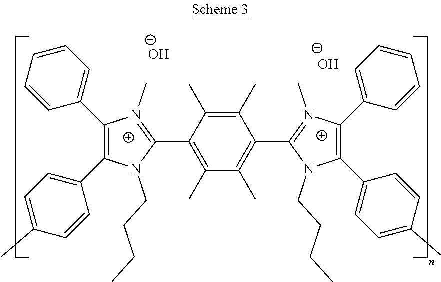

This “methyl-butyl” polymer was tested under similar conditions as the “high dm” polymer from the previous Examples. In comparison, it showed improved aliphatic character for improved activity against non-enveloped viruses. Comparable function against Gram-bacteria, Gram+ bacteria, and enveloped viruses. Comparable ability to be cast as films from low-boiling and/or non-toxic solvents. Comparable optical clarity, clearness & transparence. Improved consistency of charge density lot to lot. Reduced water uptake (or ‘water uptake to charge’ ratio) for improved durability on surfaces exposed to water or frequently washed surfaces.

While illustrative embodiments have been illustrated and described, it will be appreciated that various changes can be made therein without departing from the spirit and scope of the invention.

1. A method of inhibiting microorganisms on a surface, comprising applying to the surface a polymer comprising a repeating unit, the repeating unit comprising:
(i) a benzimidazolium-containing moiety of Formula (I): 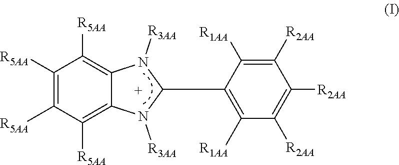
wherein:
R_{1AA} is independently selected from the group consisting of H, methyl, trifluoromethyl, alkyl, perfluoroalkyl, heteroalkyl, alkoxy, perfluoroalkoxy, halo, aryl, heteroaryl group and a polymer;
R_{2AA} is independently selected from the group consisting of hydrogen, any group, and a polymer;
R_{3AA} is independently selected from the group consisting of methyl, trifluoromethyl, alkyl, perfluoroalkyl, heteroalkyl, aryl, aralkyl, a polymer, and no group; and
R_{5AA} is independently selected from the group consisting of hydrogen, any group, and a polymer;
wherein at least one of R_{1AA}, R_{2AA}, R_{3AA}, and R_{5AA}, is a polymer;
(ii) an imidazolium-containing moiety having Formula (II): 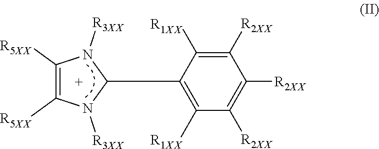
wherein:
R_{1XX} is independently selected from the group consisting of H, methyl, trifluoromethyl, alkyl, perfluoroalkyl, heteroalkyl, alkoxy, perfluoroalkoxy, halo, aryl, heteroaryl group and a polymer;
R_{2XX} is independently selected from the group consisting of hydrogen, any group, and a polymer;
R_{3XX} is independently selected from the group consisting of methyl, trifluoromethyl, alkyl, perfluoroalkyl, heteroalkyl, aryl, aralkyl, a polymer, and no group; and
R_{5XX} is independently selected from the group consisting of hydrogen, any group, and a polymer;
wherein at least one of R_{1XX}, R_{2XX}, R_{3XX}, and R_{5XX}, is a polymer, or
any combination thereof.

2. The method of claim 1, further comprising exposing the surface to the microorganism and killing the microorganism present on the surface.

3. The method of claim 1, further comprising inhibiting adhesion of the microorganism on the surface.

4. The method of claim 1, wherein when the surface is exposed to the microorganism, the number of microorganisms on the surface is reduced by greater than 99% after a duration of 24 hours.

5. The method of claim 1, wherein the microorganism comprises a fungus, a bacterium, a virus, or any combination thereof.

6. The method of claim 5, wherein when the surface is exposed to a fungus, the number of the fungus is reduced by greater than 99% after a duration of 24 hours.

7. The method of claim 5, wherein the fungus is selected from Candida spp., Cryptococcus spp., Blastomyces spp., Coccidioides spp., Aspergillus spp., Emmonsia spp., Chrysosporium spp., Fonsecaea spp., Trychophyon spp., Histoplasma spp., Ajellomyces spp., Pneumocytis spp., Microsporum spp., Magnaporthe spp., Fusarium spp., Sporothrix spp., Cyberlindnera spp., Mucormycetes spp., Epidermophyton spp., Trichosporon spp., Paracoccidioides spp., Talaromyces spp., Uncinocarpus reesii, and Aphanoascus spp.

8. The method of claim 5, wherein when the surface is exposed to a bacterium, the number of the bacterium is reduced by greater than 99% after a duration of 24 hours.

9. The method of claim 5, wherein the bacterium is a gram-positive bacterium.

10. The method of claim 5, wherein the bacterium is a gram-negative bacterium.

11. The method of claim 5, wherein the microorganism is a pathogenic bacterium.

12. The method of claim 11, wherein the pathogenic bacterium is selected from Escherichia coli, Staphylococcus aureus, Campylobacter spp., Legionella, Salmonella spp., Shigella spp., Tsukamurella, Leptospires, Mycobacterium, Pseudomonas aeruginosa, Aeromonas spp., Vibrio spp., Yersinia, Bacillus spp., Enterobacter sakazakii, Burkholderia pseudomallei, Acinetobacter spp., Helicobacter pylori, Klebsiella spp., clostridium spp., and Streptococci.

13. The method of claim 5, wherein when the surface is exposed to a virus, the number of the virus is reduced by greater than 99% after a duration of 24 hours.

14. The method of claim 5, wherein the virus is selected from alphacoronaviruses, betacoronaviruses (e.g., SARS-Cov2), deltacoronaviruses, gammacoronaviruses, and toroviruses; influenza virus A, influenza virus B, influenza virus C, influenza virus D, hepatitis A virus, hepatitis B virus, simplex viruses, cytomegaloviruses, mamastroviruses, mastadenoviruses, poxviruses, hepadnaviruses, asfarviridae, flaviviruses, alphaviruses, togaviruses, paramyxoviruses, rhabdovirus, bunyaviruses, filoviruses, retroviridae, coxsackieviruses (enveloped and non-enveloped), rotaviruses, polioviruses, calciviruses, orthopoxviruses, polyomaviruses, vesiculoviruses, varicelloviruses, phleboviruses, alphatorqueviruses, noroviruses, rubulaviruses, spumaviruses, rubulaviruses, hantaviruses, sapoviruses, saliviruses, rubiviruses, rosaviruses, lyssaviruses, enteroviruses, arenaviruses, orthobunyaviruses, parapoxviruses, henipaviruses, molluscipoxviruses, polyomaviruses, cardioviruses, morbilviruses, marburgviruses, deltaretroviruses, orthopneumoviruses, erythroviruses, respiroviruses, alphapapillomavirus, mupapillomaviruses, rhadinoviruses, roseoloviruses, deltaviruses, hepeviruses, hepaciviruses, orthohepadnaviruses, henipaviruses, pegiviruses, lymphocryptoviruses, ebolaviruses, nairoviruses, thogotoviruses, cosaviruses, seadornaviruses, kobuviruses, dependoviruses, and any combination thereof.

15. The method of claim 1, wherein the polymer comprises a counterion selected from the group consisting of iodide, triiodide, hydroxide, chloride, bromide, fluoride, cyanide, acetate, carbonate, nitrate, sulfate, phosphate, triflate, tosylate, bisulfate, bicarbonate, hydrogen phosphate, and dihydrogen phosphate.

16. The method of claim 1, wherein the benzimidazolium-containing moiety of Formula (I), the imidazolium-containing moiety having Formula (II), or both are each independently in a main chain of the polymer.

17. The method of claim 1, wherein the benzimidazolium-containing moiety of Formula (I), the imidazolium-containing moiety having Formula (II), or both are each independently in a pendant group of the polymer.

18. The method of claim 1, wherein the benzimidazolium-containing moiety of Formula (I), the imidazolium-containing moiety having Formula (II), or both are each independently part of a crosslink of the polymer.

19. The method of claim 1, wherein the polymer comprises a polymer of Formula (III): 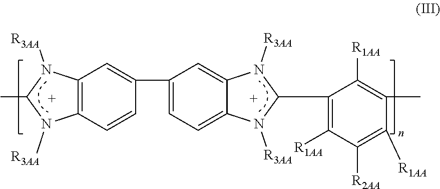
wherein R_{1AA} and R_{3AA} are methyl groups and R_{2AA} is hydrogen.

20. The method of claim 1, wherein the repeating unit comprises a benzimidazolium-containing moiety of Formulas (IV)-(VIII): 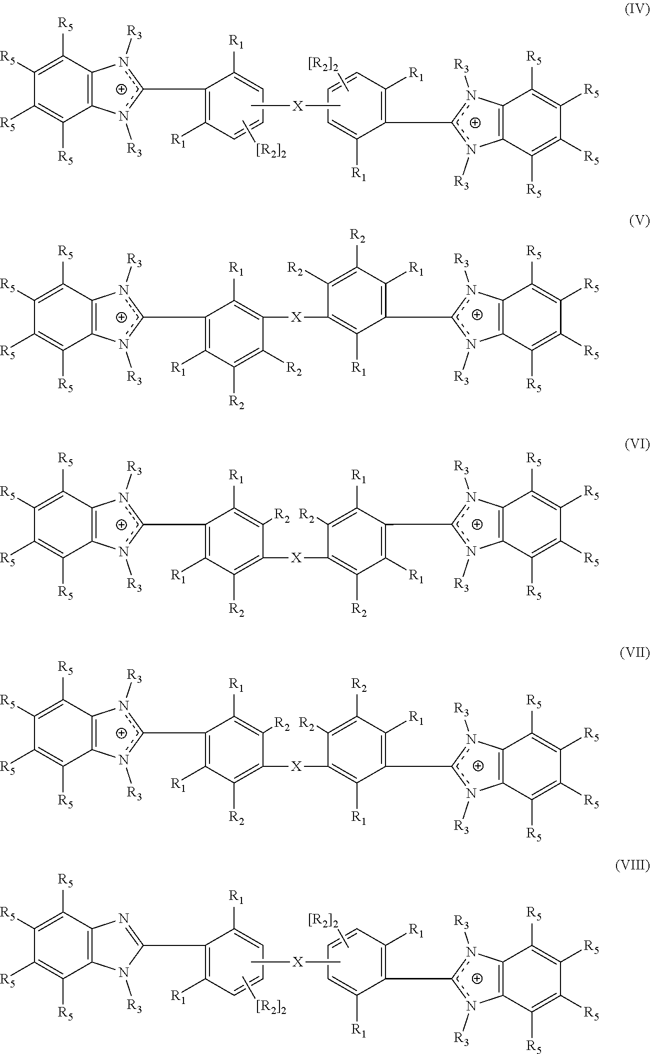
wherein:
R₁ is independently selected from the group consisting of H, methyl, trifluoromethyl, alkyl, perfluoroalkyl, heteroalkyl, alkoxy, perfluoroalkoxy, halo, aryl, heteroaryl group and a polymer;
R₂ is independently selected from the group consisting of hydrogen, any group, and a polymer;
R₃ is independently selected from the group consisting of methyl, trifluoromethyl, alkyl, perfluoroalkyl, heteroalkyl, aryl, aralkyl, and a polymer;
R₅ is independently selected from the group consisting of hydrogen, any group, and a polymer;
wherein at least one of R₁, R₂, R₃, and R₅, is a polymer; and
X is independently selected from the group consisting of alkylene, perfluoroalkylene, heteroalkylene, arylene, aralkylene, and no group.

21. The method of claim 1, wherein the polymer comprises a polymer of Formula (IX): 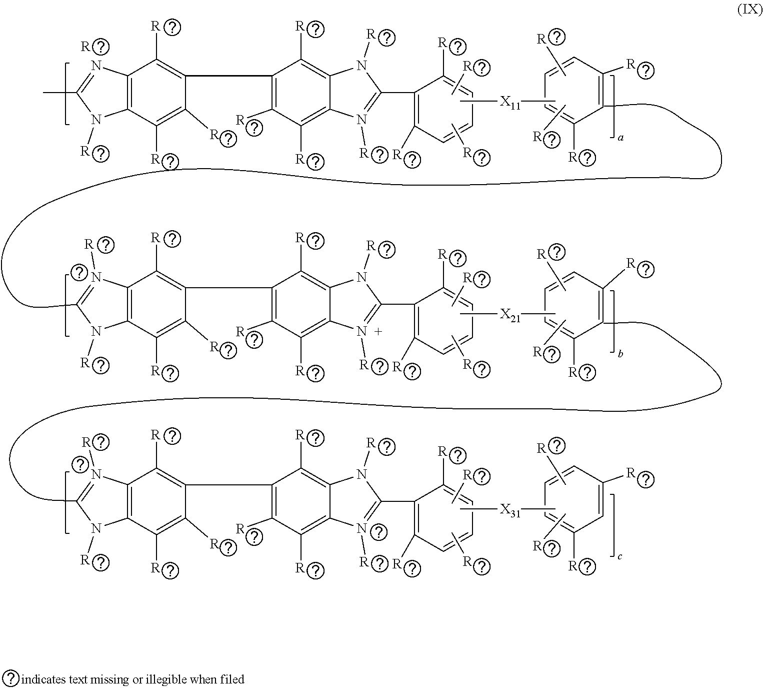
wherein:
R₁₁ₐ, R_{11b}, R_{11c}, R_{11d}, R₂₁ₐ, R_{21b}, R_{21c}, R_{21d}, R₃₁ₐ, R_{31b}, R_{31c}, and R_{31d} are each independently selected from H, C₁₋₆ alkyl, C₁₋₆ haloalkyl, C₁₋₆ alkoxy, C₁₋₆ haloalkoxy, halo, aryl, and heteroaryl;
R₁₂ₐ, R_{12b}, R_{12c}, R_{12d}, R₂₂ₐ, R_{22b}, R_{22c}, R_{22d}, R₃₂ₐ, R_{32b}, R_{32c}, and R₃₂ₐ are each independently selected from H, C₁₋₆ alkyl, C₁₋₆ haloalkyl, C₁₋₆ alkoxy, C₁₋₆ haloalkoxy, halo, aryl, and heteroaryl;
R₁₃ₐ, R_{13b}, R₁₄ₐ, and R_{14b} are each independently selected from absent, C₁₋₆ alkyl, C₁₋₆ haloalkyl, C₁₋₆ alkoxy, C₁₋₆ haloalkoxy, halo, aryl, and heteroaryl, provided that two of R₁₃ₐ, R_{13b}, R₁₄ₐ, and R_{14b} are absent, and the remaining two are present and independently selected from C₁₋₆ alkyl, C₁₋₆ haloalkyl, C₁₋₆ alkoxy, C₁₋₆ haloalkoxy, halo, aryl, and heteroaryl,
R₂₃ₐ, R_{23b}, R₂₄ₐ, and R_{24b} are each independently selected from absent, C₁₋₆ alkyl, C₁₋₆ haloalkyl, C₁₋₆ alkoxy, C₁₋₆ haloalkoxy, halo, aryl, and heteroaryl, provided that one of R₂₃ₐ, R_{23b}, R₂₄ₐ and R_{24b} is absent, and the remaining three are present and independently selected from C₁₋₆ alkyl, C₁₋₆ haloalkyl, C₁₋₆ alkoxy, C₁₋₆ haloalkoxy, halo, aryl, and heteroaryl;
R₃₃ₐ, R_{33b}, R₃₄ₐ, and R_{34b} are each independently selected from C₁₋₆ alkyl, C₁₋₆ haloalkyl, C₁₋₆ alkoxy, C₁₋₆ haloalkoxy, halo, aryl, and heteroaryl;
R₁₅ₐ, R_{15b}, R_{15c}, R_{15d}, R₁₅ₑ, R_{15f}, R₂₅ₐ, R_{25b}, R_{25c}, R_{25d}, R₂₅ₑ, R_{25f}, R₃₅ₐ, R_{35b}, R_{35c}, R_{35d}, R₃₅ₑ, and R_{35f} are each independently selected from H, C₁₋₆ alkyl, C₁₋₆ haloalkyl, C₁₋₆ alkoxy, C₁₋₆ haloalkoxy, halo, aryl, and heteroaryl;
X₁₁, X₂₁, and X₃₁ are each independently selected from the group consisting of absent, C₁₋₆ alkylene, C₁₋₆ haloalkylene, arylene, and heteroarylene; and
a, b, and c are mole percentages, wherein
a is from 0 mole percent to 45 mole percent,
b+c is 55 mole percent to 100 mole percent,
b and c are each more than 0 percent, and
a+b+c=100%.

22. The method of claim 1, wherein the repeating unit comprises an imidazolium-containing moiety of Formula (X): 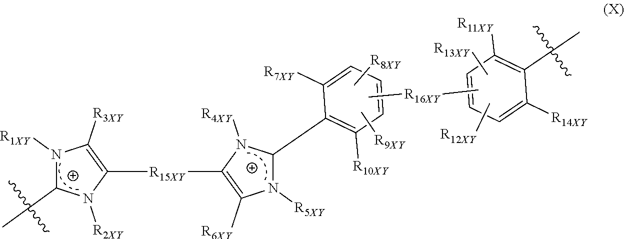
wherein:
R_{1XY}, R_{2XY}, R_{4XY}, and R_{6XY} are each independently selected from absent, alkyl, perfluoroalkyl, heteroalkyl, aryl, and aralkyl;
provided that
at least one of R_{1XY} and R_{2XY} is selected from alkyl, perfluoroalkyl, heteroalkyl, aryl, and aralkyl,
when one of R_{1XY} and R_{2XY} is absent, the imidazolyl group to which the absent R_{1XY} or R_{2XY} is connected is charge-neutral; and
at least one of R_{4XY} and R_{5XY} is selected from alkyl, perfluoroalkyl, heteroalkyl, aryl, and aralkyl; and
when one of R_{4XY} and R_{5XY} is absent, the imidazolyl group to which the absent R_{4XY} or R_{5XY} is connected is charge neutral;
R_{3XY} and R_{6XY} are each independently selected from alkyl, perfluoroalkyl, heteroalkyl, aryl, aralkyl, and heteroaryl;
R_{15XY} is selected from alkylene, perfluoroalkylene, heteroalkylene, arylene, aralkylene, and heteroarylene, each optionally substituted with 1, 2, 3, or 4 substituents independently selected from alkyl, perfluoroalkyl, heteroalkyl, and halo;
R_{16XY} is selected from a bond, arylene, and heteroarylene, wherein said arylene and heteroarylene is each optionally substituted with 1, 2, 3, or 4 substituents independently selected from alkyl, perfluoroalkyl, heteroalkyl, and halo;
R_{7XY}, R_{10XY}, R_{11XY}, and R_{14XY} are each independently selected from H, alkyl, perfluoroalkyl, and heteroalkyl; and
R_{8XY}, R_{9XY}, R_{12XY}, and R_{13XY} are each independently selected from hydrogen, alkyl, perfluoroalkyl, and heteroalkyl.

23. The method of claim 1, wherein the repeating unit comprises a moiety of Formula (XI)

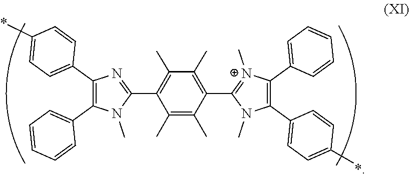

24. The method of claim 1, wherein the repeating unit comprises a moiety of Formula (XII)

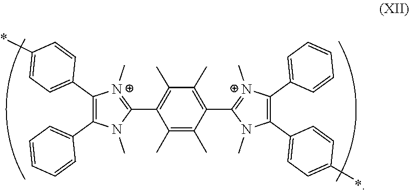

25. The method of claim 1, wherein the repeating unit comprises a moiety of Formula (XIII)

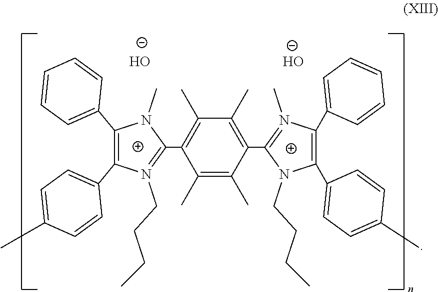

26. The method of claim 1, wherein the polymer is in the form of a coating or a membrane.

27. The method of claim 1, wherein the polymer is in the form of spun fibers, electrospun fibers, solution cast coating, extruded fibers, nanofibers, or any combination thereof.

28. The method of claim 1, wherein the polymer is in the form of a microporous film, nanoporous film, non-woven sheet, woven sheet, mat, fabric, or any combination thereof.

29. The method of claim 1, wherein the polymer is in a blend.

30. The method of claim 29, wherein the blend is a polymer blend.

31. The method of claim 1, wherein the polymer is a graft copolymer, an interpenetrating network, an aerogel, or a hydrogel.

32. The method of claim 1, wherein the surface comprises a surface of a medical device or a portion thereof.

33. The method of claim 32, wherein the medical device is selected from an urinary catheter, a percutaneous catheter, a central venous catheter, a vascular access device, a heart valve, a stent, a vascular prosthesis, a skeletal joint, a dental filling, a dental implant, an oxygen transport membrane, a suture, an intravenous delivery site, a drug delivery catheter, a drain, a gastric feeding tube, a tracheotomy tube, a contact lens, an intraocular lens, an orthopedic implant, a neuro-stimulation lead, a pacemaker lead, a blood bag, an air filter, and a drug diffusion matrix (e.g., membranes, films, rods, beads, or any combination thereof).
